(19) United States
(12) Patent Application Publication
(10) Pub. No.: US 2025/0125051 A1
(43) Pub. Date: Apr. 17, 2025

(54) GENOMIC ORIGIN, FRAGMENTOMICS, AND TRANSCRIPTIONAL CORRELATION OF LONG CELL-FREE DNA

(71) Applicant: Centre for Novostics, Shatin (HK)

(72) Inventors: Yuk-Ming Dennis Lo, Homantin (CN); Kwan Chee Chan, Jordan (CN); Peiyong Jiang, Pak Shek Kok (CN); Huiwen Che, Pak Shek Kok (CN)

(21) Appl. No.: 18/914,944

(22) Filed: Oct. 14, 2024

Related U.S. Application Data
(60) Provisional application No. 63/544,014, filed on Oct. 13, 2023.

(51) Int. Cl. G16H 50/20; C12Q 1/44; G16B 25/10; G16B 30/10; G16B 40/20; G16H 50/70

(52) U.S. Cl. CPC G16H 50/20; C12Q 1/44; G16B 25/10; G16B 30/10; G16B 40/20; G16H 50/70; G01N 2333/922

(57) ABSTRACT

Methods, apparatuses, and systems analyze cfDNA fragments in a biological sample. For example, a technique can detect cancer using aggregated amounts of cfDNA fragments ending at three or more various positions around different types of CpG sites. Another technique can detect cancer using an amount of cfDNA fragments at a set of open chromatin regions (OCRs). Another technique can estimate an expression level of a gene using an abundance of long cfDNA fragments at the gene. Another technique can identify open chromatin regions using an abundance of long cfDNA fragments. Another technique can determine a fractional concentration of cell-free DNA of a first tissue type using an abundance of long cfDNA fragments at a set of OCRs. Another technique can classify an activity of a nuclease using an abundance of long cfDNA fragments.

### CROSS-REFERENCES TO RELATED APPLICATIONS

The present application claims priority from and is a non-provisional application of U.S. Provisional Application No. 63/544,014, entitled “Genomic Origin, Fragmentomics, And Transcriptional Correlation of Long Cell-Free DNA” filed Oct. 13, 2023, the entire contents of which are herein incorporated by reference for all purposes.

### BACKGROUND

The modal size of circulating cell-free DNA in pregnancy has been reported to be at approximately 166 bp (Lo et al. Sci Transl Med. 2010; 2:61ra91). However, the biological properties of long cfDNA molecules (e.g., >500 bp) remain largely unknown. The use of long cell-free DNA molecules for detecting cancer, estimating nuclease activity, identifying open chromatin regions, estimating a fraction of clinically-relevant DNA, predicting genetic expression level, and other applications are discussed herein.

### BRIEF SUMMARY

Methods and systems described herein using cell-free DNA (cfDNA) fragments to analyze a biological sample. Various types of measurements are described, some of which use sizes of cfDNA fragments in the biological sample. Some techniques can detect cancer. For example, a technique can detect cancer using aggregated amounts of cfDNA fragments ending at three or more various positions around different types of CpG sites. Another technique can detect cancer using an amount of cfDNA fragments at a set of open chromatin regions (OCRs). Another technique can estimate an expression level of a gene using an abundance of long cfDNA fragments at the gene. Another technique can identify open chromatin regions using an abundance of long cfDNA fragments. Another technique can determine a fractional concentration of cell-free DNA of a first tissue type using an abundance of long cfDNA fragments at a set of OCRs. Another technique can classify an activity of a nuclease using an abundance of long cfDNA fragments. Aspects of implementation can be shared across the different techniques.

One general aspect includes a method of detecting cancer. The method of detecting cancer can include aligning sequence reads of cell-free DNA molecules from a biological sample of a subject to a reference genome to determine genomic coordinates of ends of the cell-free DNA molecules. The biological sample can be blood, plasma, or serum. The method can also include measuring a proportion of a group of the cell-free DNA molecules within an open chromatin region (OCR) of a set of OCRs. The method can also include determining a value of a parameter using the proportion. The method can also include comparing the value of the parameter to a reference value. The method also include determining, using the comparison, a classification of cancer in the subject.

Another general aspect includes a method of estimating an expression level of a gene. The method also includes measuring sizes of cell-free DNA molecules. The method can also include identifying a group of cell-free DNA molecules corresponding to locations in a genomic region within the gene. The method can also include measuring a first amount of the group of cell-free DNA molecules having a size greater than a first cutoff value. The method can also include measuring a second amount of the group of cell-free DNA molecules having a size less than a second cutoff value that is equal to or less than the first cutoff value. The method can also include determining a value of a parameter using the first amount and the second amount, where the parameter reflects a relative amount between the first amount and the second amount. The method can also include comparing the value of the parameter to one or more calibration values, where the one or more calibration values are determined from samples with known expression levels of the gene. The method can also include estimating the expression level for the gene using the comparison.

One general aspect includes a method of detecting cancer. The method of detecting cancer can also include aligning sequence reads of cell-free DNA molecules from a biological sample of a subject to a reference genome to determine genomic coordinates of ends of the cell-free DNA molecules. The method can also include, for each CpG site of a plurality of CpG sites: measuring, using the genomic coordinates, a first amount of cell-free DNA molecules ending at a first position within a window around the CpG site, thereby determining a plurality of first amounts; measuring, using the genomic coordinates, a second amount of cell-free DNA molecules ending at a second position within the window around the CpG site, thereby determining a plurality of second amounts; and measuring, using the genomic coordinates, a third amount of cell-free DNA molecules ending at a third position within the window around the CpG site, thereby determining a plurality of third amounts. The window can include positions −5 to +5 relative to the CpG site. The plurality of CpG sites may include one or more hypermethylated CpG sites and one or more hypomethylated CpG sites. The method can also include aggregating the plurality of first amounts to obtain a first aggregated amount. The method can also include aggregating the plurality of second amounts to obtain a second aggregated amount. The method can also include aggregating the plurality of third amounts to obtain a third aggregated amount. The method can also include determining a value of a parameter using the first aggregated amount, the second aggregated amount, and the third aggregated amount. The method can also include comparing the value of the parameter to a reference value. The method can also include determining, using the comparison, a classification of cancer in the subject.

One general aspect includes a method of classifying an activity level of a first nuclease in a biological sample. The method can also include measuring sizes of cell-free DNA molecules in the biological sample. The method can also include measuring one or more amounts of cell-free DNA molecules corresponding to one or more sizes. The one or more sizes can be greater than a cutoff value. The method can also include normalizing the one or more amounts using a first amount of cell-free DNA molecules at a first size less than the cutoff value, thereby obtaining one or more normalized amounts. The method can also include determining a value of a parameter using the one or more normalized amounts. The method can also include comparing the value of the parameter to a reference value. The reference value can be obtained using a sample having known activity levels of a set of nucleases, where a known activity level of the first nuclease is below normal and the known activity level of one or more other nucleases is within a normal range in the sample. The method can also include classifying the activity level of the first nuclease using the comparison.

One general aspect includes a method of identifying an open chromatin region (OCR) in a genome of a subject. The method can also include measuring sizes of cell-free DNA molecules in a biological sample of the subject. The method can also include aligning sequence reads of cell-free DNA molecules to a reference genome to determine genomic coordinates of an end of each cell-free DNA molecule. The method can also include for each position of a plurality of positions in a genomic region: measuring a first amount of cell-free DNA molecules having a size greater than a first cutoff value and having an end at the position, measuring a second amount of cell-free DNA molecules having a size less than a second cutoff value and having an end at the position, determining a value of a parameter using the first amount and the second amount, where the parameter reflects a relative amount between the first amount and the second amount, and comparing the value of the parameter to a reference value to identify a set of positions where the value of the parameter exceeds the reference value. The second cutoff value can be equal to or less than the first cutoff value. The OCR can be identified using the set of positions.

One general aspect includes a method of determining a fractional concentration of cell-free DNA of a first tissue type in a biological sample of a subject. The method can include measuring sizes of cell-free DNA molecules in the biological sample of a subject. The method can also include identifying a group of cell-free DNA molecules within an open chromatin region (OCR) of a set of OCRs. The method can also include measuring a first amount of the group of cell-free DNA molecules having a size greater than a first cutoff value. The method can also include measuring a second amount of the group of cell-free DNA molecules having a size less than a second cutoff value that is equal to or less than the first cutoff value. The method can also include determining a value of a parameter using the first amount and the second amount. The parameter can reflect a relative amount between the first amount and the second amount. The method can also include comparing the value of the parameter with a calibration value obtained from one or more calibration samples having known fractional concentrations of cell-free DNA of the first tissue type. The method can also include estimating the fractional concentration of cell-free DNA of the first tissue type using the comparison.

These and other embodiments of the disclosure are described in detail below. For example, other embodiments are directed to systems, devices, and computer readable media associated with methods described herein.

A better understanding of the nature and advantages of embodiments of the present disclosure may be gained with reference to the following detailed description and the accompanying drawings.

### BRIEF DESCRIPTION OF THE DRAWINGS

FIG. 1 shows a table of pooled sequenced plasma DNA molecules from previous studies using the PacBio and ONT platforms.

FIG. 2A shows a distribution of long and short molecules from chromosome 10 in human plasma samples as determined using SMRT sequencing for healthy subjects. FIG. 2B shows a distribution of long and short molecules from chromosome 10 in human plasma samples from 31 first-trimester pregnancies as determined using ONT sequencing.

FIG. 3 shows genome-wide representation between long and short pooled DNA molecules from healthy individuals with sequencing on the PacBio platform.

FIG. 4 shows genome-wide representation between long and short pooled DNA molecules from pregnant individuals with sequencing on the ONT platform.

FIG. 5A shows the abundance of SMRT long and short molecules on gene bodies of unexpressed and housekeeping genes for healthy individuals and pregnant subjects. FIG. 5B shows the abundance of ONT long and short molecules on gene bodies of unexpressed and housekeeping genes for pregnant subjects.

FIG. 6 shows the abundance of SMRT long and short molecules on gene bodies of expression-stratified gene groups for healthy individuals.

FIG. 7A shows the abundance of long and short molecules on gene bodies of expression-stratified gene groups for the first-trimester individuals sequenced on the PacBio platform. FIG. 7B shows the abundance of long and short molecules on gene bodies of expression-stratified gene groups for the first-trimester individuals sequenced on the ONT platform.

FIG. 8A shows the abundance of SMRT long and short molecules on gene bodies of gene groups having varying median expression levels across tissues for healthy individuals using the PacBio platform. FIG. 8B shows the abundance of SMRT long and short molecules on gene bodies of gene groups having varying median expression levels across tissues for healthy individuals using the ONT platform.

FIG. 9A shows normalized end frequencies of SMRT long and short molecules pooled from plasma of healthy individuals at TSSs of expression-stratified gene groups EXP1 to EXP5, corresponding to low to high expression. FIG. 9B shows normalized end frequencies of ONT long and short molecules pooled from plasma of pregnant individuals at TSSs of expression-stratified gene groups EXP1 to EXP5, corresponding to low to high expression.

FIG. 10A shows the abundance of long (>600) and short (<200) molecules on gene bodies of expression-stratified gene groups for healthy individuals. FIG. 10B shows normalized end frequencies of long (>600) and short (<200) molecules pooled from plasma of healthy individuals at TSSs of expression-stratified gene groups EXP1 to EXP5, corresponding to low to high expression.

FIG. 11 is a flowchart of an example process 1100 of predicting an expression level of a gene.

FIG. 12A shows normalized end frequencies of SMRT long and short molecules pooled from plasma of healthy individuals at DHSs. FIG. 12B shows normalized end frequencies of SMRT long and short molecules pooled from plasma of healthy individuals at CTCFs. FIG. 12C shows normalized end frequencies of ONT long and short molecules pooled from plasma of pregnant individuals at DHSs. FIG. 12D shows normalized end frequencies of ONT long and short molecules pooled from plasma of pregnant individuals at CTCF.

FIG. 13A shows normalized end frequencies of long (>600 bp) and short (<200 bp) molecules pooled from plasma of healthy individuals at DHSs. FIG. 13B shows normalized end frequencies of long (>600 bp) and short (<200 bp) molecules pooled from plasma of healthy individuals at CTCFs.

FIG. 14 is a flowchart of an example process 1600 for identifying an open chromatin region (OCR) in a genome of a subject.

FIG. 15A is an example for the correlation between the ratio of long (>500 bp) to short (<500 bp) cfDNA originating from the OCRs and tumor DNA fraction. FIG. 15B shows a plot between the ratio of long (>500 bp) to short (<500 bp) cfDNA originating from the OCRs and fetal DNA fraction.

FIG. 16 shows normalized end frequencies of long and short molecules pooled from plasma of healthy individuals at tissue-specific DHSs for liver, myeloid, lymphoid, and gastrointestinal tissues.

FIG. 17 is a flowchart of an example process 1600 of determining a fractional concentration of cell-free DNA of a first tissue type in a biological sample of a subject.

FIG. 18 shows a table of SMRT sequencing data using plasma samples from 20 healthy individuals, 19 hepatitis B virus (HBV) carriers and 48 patients with hepatocellular carcinoma (HCC).

FIG. 19A shows a ratio of an abundance of long cfDNA fragments to short cfDNA fragment at a set of genes that are upregulated in CRC for patients with colon adenocarcinoma (COAD) and healthy controls. FIG. 19B shows a ratio of an abundance of long cfDNA fragments to short cfDNA fragment at a set of genes that are upregulated in HCC for HCC patients, HBV carriers, and healthy controls.

FIG. 20 shows a comparison of the abundance of long and short molecules at 5,000 HCC-associated genes among healthy individuals, HBV carriers and patients with HCC.

FIG. 21 shows ROCs of long molecule abundance for distinguishing individuals without HCC, including healthy subjects and HBV carriers, and with HCC.

FIG. 22A shows how the OCR regions and the non-OCR regions were defined. FIG. 22B shows the proportion of long cfDNA, short cfDNA, long+short cfDNA, and all cfDNA at OCRs.

FIG. 23A shows the proportion of long cfDNA, short cfDNA, long+short cfDNA, and all cfDNA at non-OCRs. FIG. 23B shows ROC curves using proportions of fragment ends in OCRs.

FIG. 24 is a flowchart of a method for detecting cancer using cfDNA at open chromatin regions.

FIG. 25 shows cleavage profiles of pooled PacBio molecules from healthy subjects at commonly methylated (left panel) and commonly unmethylated (right panel) CpGs.

FIG. 26A shows cleavage profiles surrounding all autosomal CpGs for short cfDNA molecules for healthy, HBV, and HCC subjects. Each line represents one sample. A cleavage window of 11 bases is shown. Position 0 and 1 indicate cytosine and guanine, respectively. FIG. 26B shows cleavage profiles surrounding all autosomal CpGs for long cfDNA molecules for healthy, HBV, and HCC subjects.

FIG. 27 shows a box plot of CGN/NCG motif ratios for long and short molecules for healthy, HBV, and HCC subjects.

FIG. 28 shows a box plot of ending ratios between aggregating positions −4, −2, 1, and 4 and position −1 for long, short, and long+short molecules.

FIG. 29 shows Receiver Operating Characteristics (ROCs) curves for distinguishing patients with HCC from non-HCC subjects for short, long, and long+short cfDNA fragments using an ending ratio of aggregating positions −4, −2, 1, and 4 and position −1.

FIG. 30A shows a plot of probabilities for discriminating between healthy, HBV, and HCC subjects for long, short, and concatenation of both using an SVM model for a window of −5 to +5 around all CpG sites. FIG. 30B shows areas under the curve for distinguishing patients with HCC from non-HCC subjects using probabilities scores in FIG. 30A.

FIG. 31A shows ROC curves for long, short, concatenated, and all molecules using positions −4 to 4 aggregated across all CpG sites. FIG. 31B shows ROC curves for long, short, concatenated, and all molecules using positions −3 to 3 aggregated across all CpG sites.

FIG. 32A shows ROC curves for long, short, concatenated, and all molecules using positions −2 to 2 aggregated across all CpG sites. FIG. 32B shows ROC curves for long, short, concatenated, and all molecules using positions 4, −2, −1, 1 and 4 aggregated across all CpG sites.

FIG. 33 shows ROC curves for long, short, concatenated, and all molecules using positions −4, −2, and 4 aggregated across all CpG sites.

FIGS. 34A and 34B show results for differentiating HCC vs. non-HCC for all 26,752,699 autosome CpGs, using long, short, long+short, and all cfDNA fragments and a first sum over position −4 and −2, and a second value for position −1. FIGS. 35A and 35B show results for differentiating HCC vs. non-HCC for all 26,752,699 autosome CpGs, using long, short, long+short, and all cfDNA fragments and a first sum over position −3 and 0, and a second value for position −1. FIGS. 36A and 36B show results for differentiating HCC vs. non-HCC for all 26,752,699 autosome CpGs, using long, short, long+short, and all cfDNA fragments and a first sum over position −4, −2, 1 and 4, and a second value for position 0. FIGS. 37A and 37B show results for differentiating HCC vs. non-HCC for all 26,752,699 autosome CpGs, using long, short, long+short, and all cfDNA fragments and a first sum over position −4, −2, 1 and 4, and a second value for position −1 and 0.

FIGS. 38A and 38B show results for differentiating HCC vs. non-HCC for randomly selected 15 million CpGs, using long, short, long+short, and all cfDNA fragments and a first sum over position −4, −2, 1 and 4, and a second value for position −1. FIGS. 39A and 39B show results for differentiating HCC vs. non-HCC for randomly selected 15 million CpGs, using long, short, long+short, and all cfDNA fragments and a first sum over position −4 and −2, and a second value for position −1. FIGS. 40A and 40B show results for differentiating HCC vs. non-HCC for randomly selected 15 million CpGs, using long, short, long+short, and all cfDNA fragments and a first sum over position −3 and 0, and a second value for position −1. FIGS. 41A and 41B show results for differentiating HCC vs. non-HCC for randomly selected 15 million CpGs, using long, short, long+short, and all cfDNA fragments and a first sum over position −4, −2, 1 and 4, and a second value for position 0. FIGS. 42A and 42B show results for differentiating HCC vs. non-HCC for randomly selected 15 million CpGs, using long, short, long+short, and all cfDNA fragments and a first sum over position −4, −2, 1 and 4, and a second value for position −1 and 0.

FIGS. 43A and 43B show results for differentiating HCC vs. non-HCC for randomly selected 10 million CpGs, using long, short, long+short, and all cfDNA fragments and a first sum over position −4, −2, 1 and 4, and a second value for position −1. FIGS. 44A and 44B show results for differentiating HCC vs. non-HCC for randomly selected 10 million CpGs, using long, short, long+short, and all cfDNA fragments and a first sum over position −4 and −2, and a second value for position −1. FIGS. 45A and 45B show results for differentiating HCC vs. non-HCC for randomly selected 10 million CpGs, using long, short, long+short, and all cfDNA fragments and a first sum over position −3 and 0, and a second value for position −1. FIGS. 46A and 46B show results for differentiating HCC vs. non-HCC for randomly selected 10 million CpGs, using long, short, long+short, and all cfDNA fragments and a first sum over position −4, −2, 1 and 4, and a second value for position 0. FIGS. 47A and 47B show results for differentiating HCC vs. non-HCC for randomly selected 10 million CpGs, using long, short, long+short, and all cfDNA fragments and a first sum over position −4, −2, 1 and 4, and a second value for position −1 and 0.

FIGS. 48A and 48B show results for differentiating HCC vs. non-HCC for randomly selected 5 million CpGs, using long, short, long+short, and all cfDNA fragments and a first sum over position −4, −2, 1 and 4, and a second value for position −1. FIGS. 49A and 49B show results for differentiating HCC vs. non-HCC for randomly selected 5 million CpGs, using long, short, long+short, and all cfDNA fragments and a first sum over position −4 and −2, and a second value for position −1. FIGS. 50A and 50B show results for differentiating HCC vs. non-HCC for randomly selected 5 million CpGs, using long, short, long+short, and all cfDNA fragments and a first sum over position −3 and 0, and a second value for position −1. FIGS. 51A and 51B show results for differentiating HCC vs. non-HCC for randomly selected 5 million CpGs, using long, short, long+short, and all cfDNA fragments and a first sum over position −4, −2, 1 and 4, and a second value for position 0. FIGS. 52A and 52B show results for differentiating HCC vs. non-HCC for randomly selected 5 million CpGs, using long, short, long+short, and all cfDNA fragments and a first sum over position −4, −2, 1 and 4, and a second value for position −1 and 0.

FIGS. 53A and 53B show results for differentiating HCC vs. non-HCC for randomly selected 1 million CpGs, using long, short, long+short, and all cfDNA fragments and a first sum over position −4, −2, 1 and 4, and a second value for position −1. FIGS. 54A and 54B show results for differentiating HCC vs. non-HCC for randomly selected 1 million CpGs, using long, short, long+short, and all cfDNA fragments and a first sum over position −4 and −2, and a second value for position −1. FIGS. 55A and 55B show results for differentiating HCC vs. non-HCC for randomly selected 1 million CpGs, using long, short, long+short, and all cfDNA fragments and a first sum over position −3 and 0, and a second value for position −1. FIGS. 56A and 56B show results for differentiating HCC vs. non-HCC for randomly selected 1 million CpGs, using long, short, long+short, and all cfDNA fragments and a first sum over position −4, −2, 1 and 4, and a second value for position 0. FIGS. 57A and 57B show results for differentiating HCC vs. non-HCC for randomly selected 1 million CpGs, using long, short, long+short, and all cfDNA fragments and a first sum over position −4, −2, 1 and 4, and a second value for position −1 and 0.

FIGS. 58A and 58B show results for differentiating HCC vs. non-HCC for randomly selected 0.5 million CpGs, using long, short, long+short, and all cfDNA fragments and a first sum over position −4, −2, 1 and 4, and a second value for position −1. FIGS. 59A and 59B show results for differentiating HCC vs. non-HCC for randomly selected 0.5 million CpGs, using long, short, long+short, and all cfDNA fragments and a first sum over position −4 and −2, and a second value for position −1. FIGS. 60A and 60B show results for differentiating HCC vs. non-HCC for randomly selected 0.5 million CpGs, using long, short, long+short, and all cfDNA fragments and a first sum over position −3 and 0, and a second value for position −1. FIGS. 61A and 61B show results for differentiating HCC vs. non-HCC for randomly selected 0.5 million CpGs, using long, short, long+short, and all cfDNA fragments and a first sum over position −4, −2, 1 and 4, and a second value for position 0. FIGS. 62A and 62B show results for differentiating HCC vs. non-HCC for randomly selected 0.5 million CpGs, using long, short, long+short, and all cfDNA fragments and a first sum over position −4, −2, 1 and 4, and a second value for position −1 and 0.

FIG. 63 is an ROC curve for differentiating CRC vs. non-CRC for 26,752,699 autosome CpGs, using long, short, long+short, and all cfDNA fragments and a first sum over position −4, −2, 1 and 4, and a second value for position −1.

FIG. 64 is a flowchart of an example process 6300 for detecting cancer using cleavage profiles aggregated at various types of CpG sites.

FIG. 65 shows a table of samples analyzed from the wild-type (WT), 5 Dffb^{-/-}, 5 Dnase1^{-/-}, 5 Dnase1l3^{-/-}, and 5 Dnase1^{-/-} and Dnase1l3^{-/-} double knockout mice.

FIG. 66A shows pooled molecule size distributions of wild-type and nuclease-deficient mice sequenced using PacBio sequencing platform. FIG. 66B shows a boxplot of proportions of long molecules (>500 bp) originated from 2000 bp upstream and downstream of transcription starting sites for different subjects that lack certain nucleases.

FIG. 67 illustrates boxplots showing proportions of long molecules originated from 2000 bp upstream and downstream of DHSs and CTCFs for different subjects that lack certain nucleases.

FIG. 68A illustrates boxplots showing proportions of long molecules (>400 bp) originated from 2000 bp upstream and downstream of DHSs and CTCFs for different subjects that lack certain nucleases. FIG. 68B illustrates boxplots showing proportions of long molecules (>600 bp) originated from 2000 bp upstream and downstream of DHSs and CTCFs for different subjects that lack certain nucleases.

FIG. 69 shows a percent of changes in pooled long molecules (>500 bp) abundance relative to the wild-type mice on low, medium, and high expression gene groups.

FIG. 70 is a flowchart of an example process for measuring an activity of a first nuclease in a biological sample using sizes of cfDNA fragments.

FIG. 71 illustrates a measurement system according to an embodiment of the present disclosure.

FIG. 72 shows a block diagram of an example computer system usable with systems and methods according to embodiments of the present disclosure.

### TERMS

A “tissue” corresponds to a group of cells that group together as a functional unit. More than one type of cells can be found in a single tissue. Different types of tissue may consist of different types of cells (e.g., hepatocytes, alveolar cells or blood cells), but also may correspond to tissue from different organisms (mother vs. fetus) or to healthy cells vs. tumor cells. “Reference tissues” can correspond to tissues used to determine tissue-specific methylation levels. Multiple samples of a same tissue type from different individuals may be used to determine a tissue-specific methylation level for that tissue type.

A “biological sample” refers to any sample that is taken from a subject (e.g., a human or other animal), such as a pregnant woman, a person with cancer or other disorder, or a person suspected of having cancer or other disorder, an organ transplant recipient or a subject suspected of having a disease process involving an organ (e.g., the heart in myocardial infarction, or the brain in stroke, or the hematopoietic system in anemia) and contains one or more nucleic acid molecule(s) of interest (e.g., DNA and/or RNA). The biological sample can be a bodily fluid, such as blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), intraocular fluids (e.g., the aqueous humor), amniotic fluid, etc. Stool samples can also be used. In various embodiments, the majority of DNA in a biological sample (e.g., that has been enriched for cell-free DNA, such as a plasma sample obtained via a centrifugation protocol) can be cell-free, e.g., greater than 50%, 60%, 70%, 80%, 90%, 95%, or 99% of the DNA can be cell-free. A centrifugation protocol for enriching cell-free DNA from a biological sample can include, for example, centrifuging the biological sample at 1,600 g×10 minutes, obtaining the fluid part of the centrifuged sample, and re-centrifuging at for example, 16,000 g for another 10 minutes to remove residual cells. As part of an analysis of a biological sample, a statistically significant number of cell-free DNA molecules can be analyzed (e.g., to provide an accurate measurement) for a biological sample. In some embodiments, at least 1,000 cell-free DNA molecules are analyzed. In other embodiments, at least 10,000 or 50,000 or 100,000 or 500,000 or 1,000,000 or 5,000,000 cell-free DNA molecules, or more, can be analyzed. At least a same number of sequence reads can be analyzed. Any amount described herein can be any of the numbers listed above. Examples sizes of a sample can include 30, 50, 100, 200, 300, 500, 1,000, 5,000, or 10,000 or more nanograms, or 1, 2, 3, 4, 5, 6, 7, 8, 9, or 10 ml.

The term “assay” generally refers to a technique for determining a property of a nucleic acid or a sample of nucleic acids (e.g., a statistically significant number of nucleic acids), as well as a property of the subject from which the sample was obtained. An assay (e.g., a first assay or a second assay) generally refers to a technique for determining the quantity of nucleic acids in a sample, genomic identity of nucleic acids in a sample, the copy number variation of nucleic acids in a sample, the methylation status of nucleic acids in a sample, the fragment size distribution of nucleic acids in a sample, the mutational status of nucleic acids in a sample, or the fragmentation pattern of nucleic acids in a sample. Any assay known to a person having ordinary skill in the art may be used to detect any of the properties of nucleic acids mentioned herein. Properties of nucleic acids include a sequence, quantity, genomic identity, copy number, a methylation state at one or more nucleotide positions, a size of the nucleic acid, a mutation in the nucleic acid at one or more nucleotide positions, and the pattern of fragmentation of a nucleic acid (e.g., the nucleotide position(s) at which a nucleic acid fragments). The term “assay” may be used interchangeably with the term “method”. An assay or method can have a particular sensitivity and/or specificity (e.g., based on selection of one or more cutoff values), and their relative usefulness as a diagnostic tool can be measured using Receiver Operating Characteristic (ROC) Area-Under-the-Curve (AUC) statistics.

The terms “control”, “control sample”, “background sample,” “reference”, “reference sample”, “normal”, and “normal sample” may be interchangeably used to generally describe a sample that does not have a particular condition or is otherwise healthy. In an example, a no-template control (NTC) sample with contaminant DNA can be considered as a reference sample. In another example, the reference sample is a sample taken from a subject without an infection. A reference sample may be obtained from the subject, or from a database. The reference generally refers to a reference genome that is used to map sequence reads obtained from sequencing a sample from the subject. A reference genome generally refers to a haploid or diploid genome to which sequence reads from the biological sample can be aligned and compared. For a haploid genome, there is only one nucleotide at each locus. For a diploid genome, heterozygous loci can be identified, with such a locus having two alleles, where either allele can allow a match for alignment to the locus. A reference genome can be a reference microbe genome that corresponds to a particular microbe species, e.g., by including one or more microbe genomes.

A “reference genome” or “reference sequence” may be an entire genome sequence of a reference organism, one or more portions of a reference genome that may or may not be contiguous, a consensus sequence of many reference organisms, a compilation sequence based on different components of different organisms, or any other appropriate reference sequence. As examples, a reference genome/sequence can at least 1,000, 10,000, 50,000, 100,000, 500,000, 1,000,000, 5,000,000, 10,000,000, 50,000,000, 100,000,000, 500,000,000, one billions, or 3 billion nucleotides long, e.g., a full human genome or a repeat masked human genome. A reference may also include information regarding variations of the reference known to be found in a population of organisms.

The term “fragment” (e.g., a DNA or an RNA fragment), as used herein, can refer to a portion of a polynucleotide or polypeptide sequence that comprises at least 3 consecutive nucleotides. A nucleic acid fragment can retain the biological activity and/or some characteristics of the parent polypeptide. A nucleic acid fragment can be double-stranded or single-stranded, methylated or unmethylated, intact or nicked, complexed or not complexed with other macromolecules, e.g. lipid particles, proteins. A nucleic acid fragment can be a linear fragment or a circular fragment. A tumor-derived nucleic acid can refer to any nucleic acid released from a tumor cell, including pathogen nucleic acids from pathogens in a tumor cell. As part of an analysis of a biological sample, a statistically significant number of fragments can be analyzed, e.g., at least 1,000 fragments can be analyzed. As other examples, at least 5,000, 10,000 or 50,000 or 100,000 or 500,000 or 1,000,000 or 5,000,000 fragments, or more, can be analyzed, and such fragments can be randomly selected or selected according to one or more criteria.

A long fragment (also referred to as a long molecule) can refer to cell-free molecules that are longer than or equal to and longer than a first size cutoff, such as but not limited to at least 400 nt, 450 nt, 500 nt, 550 nt, 600 nt, 650 nt, 700 nt, 750 nt, 800 nt, 850 nt, 900 nt, 950 nt, 1 knt, 1.1 knt, 1.2 knt, 1.3 knt, 1.4 knt, 1.5 knt, 1.6 knt, 1.7 knt, 1.8 knt, 1.9 knt, 2 knt, 2.5 knt, 3 knt, 4 knt, 5 knt, 6 knt, 7 knt, 8 knt, 9 knt, 10 knt, 15 knt, 20 knt, 30 knt, 40 knt, 50 knt, 60 knt, 70 knt, 80 knt, 90 knt, 100 knt, 200 knt, 300 knt, 400 knt, 500 knt, or 1 Mnt.

A short fragment (also referred to as a short molecule) can refer to cell-free molecules that are shorter than or equal to and shorter than a second size cutoff, such as but not limited to less than 100 nt, 120 nt, 150 nt, 180 nt, 200 nt, 250 nt, 300 nt, 350 nt, 400 nt, 450 nt, 500 nt, 550 nt, 600 nt, 650 nt, 700 nt, 750 nt, 800 nt, 850 nt, 900 nt, 950 nt, or 1 knt. These cutoff values may apply to any methods described herein. The second size cutoff for small molecules can be the same or different than the first size cutoff used for long fragments. An example for the size cutoffs being the same is <500 for small and >500 for long. An example for the size cutoffs being different is <400 for small and >600 for long.

A “sequence read” refers to a string of nucleotides obtained from any part or all of a nucleic acid molecule. For example, a sequence read may be a short string of nucleotides (e.g., 20-150 nucleotides) sequenced from a nucleic acid fragment, a short string of nucleotides at one or both ends of a nucleic acid fragment, or the sequencing of the entire nucleic acid fragment that exists in the biological sample. A sequence read may be obtained in a variety of ways, e.g., using sequencing techniques or using probes, e.g., in hybridization arrays or capture probes as may be used in microarrays, or amplification techniques, such as the polymerase chain reaction (PCR) or linear amplification using a single primer or isothermal amplification. Example sequencing techniques include massively parallel sequencing, targeted sequencing, Sanger sequencing, sequencing by ligation, ion semiconductor sequencing, and single molecule sequencing (e.g., using a nanopore, or single-molecule real-time sequencing (e.g., from Pacific Biosciences)). Such sequencing can be random sequencing or targeted sequencing (e.g., by using capture probes hybridizing to specific regions or by amplifying certain region, both of which enrich such regions). Example probe-based techniques include real-time PCR and digital PCR (e.g., droplet digital PCR). As part of an analysis of a biological sample, a statistically significant number of sequence reads can be analyzed, e.g., at least 1,000 sequence reads can be analyzed. As other examples, at least 5,000, 10,000, 50,000, 100,000, 500,000, 1,000,000, or 5,000,000 sequence reads, or more, can be analyzed. Additionally, amounts of sequence reads determined for embodiments of the present disclosure can be at least 1,000, 5,000, 10,000, 50,000, 100,000, 500,000, 1,000,000, or 5,000,000.

The term “mapping” or “aligning” refers to a process that relates a sequence to a location or coordinate (e.g., a genomic coordinate) in a reference (e.g., a reference genome) having a known reference sequence, where the sequence is similar to the known reference sequence at the location in the reference.

“Single-molecule sequencing” refers to sequencing of a single template DNA molecule to obtain a sequence read without the need to interpret base sequence information from clonal copies of a template DNA molecule. The single-molecule sequencing may sequence the entire molecule or only part of the DNA molecule. A majority of the DNA molecule may be sequenced, e.g., greater than 50%, 55%, 60%, 65%, 70%, 75%, 80%, 85%, 90%, 95%, or 99%. A sequence read (or reads from both ends) can be aligned to a reference genome. When both ends are aligned (e.g., as part of a read of the entire fragment or for paired-ends), greater accuracy can be achieved in the alignment and a length of the fragment can be obtained. Embodiments of the present disclosure can use single-molecule sequencing.

“Clinically-relevant DNA” can refer to DNA of a particular tissue source that is to be measured, e.g., to determine a fractional concentration of such DNA or to classify a phenotype of a sample (e.g., plasma). Examples of clinically-relevant DNA are fetal DNA in maternal plasma or tumor DNA in a patient's plasma or other sample with cell-free DNA. Another example includes the measurement of the amount of graft-associated DNA in the plasma, serum, or urine of a transplant patient. A further example includes the measurement of the fractional concentrations of hematopoietic and nonhematopoietic DNA in the plasma of a subject, or fractional concentration of a liver DNA fragments (or other tissue) in a sample or fractional concentration of brain DNA fragments in cerebrospinal fluid.

The term “fractional fetal DNA concentration” is used interchangeably with the terms “fetal DNA proportion” and “fetal DNA fraction,” and refers to the proportion of fetal DNA molecules that are present in a biological sample (e.g., maternal plasma or serum sample) that is derived from the fetus (Lo et al, Am J Hum Genet. 1998; 62:768-775; Lun et al, Clin Chem. 2008; 54:1664-1672). Similarly, tumor fraction or tumor DNA fraction can refer to the fractional concentration of tumor DNA in a biological sample, or tissue fraction can refer to the fractional concentration of DNA from one or more particular tissue(s), e.g., from a transplant organ.

A “site” (also called a “genomic site”) corresponds to a single site, which may be a single base position or a group of correlated base positions, e.g., a CpG site, TSS site, DNase hypersensitivity site, or larger group of correlated base positions. A “locus” may correspond to a region that includes multiple sites. A locus can include just one site, which would make the locus equivalent to a site in that context. A region can be defined around a site, e.g., a symmetric or asymmetric region around a site. As examples, a region can include at least +/−50 bases before and after a site (e.g., 101 bases), +/−60 bases, +/−70 bases, +/−80 bases, +/−90 bases, +/−100 bases, +/−150 bases, +/−200 bases, +/−300 bases, +/−400 bases, +/−500 bases, +/−600 bases, +/−700 bases, +/−800 bases, +/−900 bases, and +/−1,000 bases. As other examples a region can be at least 100 bases, 140 bases, 147 bases, or 167 bases long. One or more regions can be analyzed, e.g., to provide a level of a pathology (e.g., cancer) or a fraction of a particular tissue. Various number of regions, sites, or loci can be analyzed, e.g., 50, 100, 200, 500, 1,000, 5,000, 10,000, 50,000, 100,000, 500,000, one million, or more. Various techniques can determine a DNA molecule is located at one or more genomic positions in a reference genome, e.g., alignment of a sequence read to the reference genome or using position-specific probes. The position determination can be to some or all of the reference genome, e.g., if only part of the genome is being analyzed. As examples, the amount of the genome analyzed can be greater than 0.01%, 0.1%, 1%, 5%, 10%, or 50%. A “cutting site” can refer to a location that DNA was cut by a nuclease, thereby resulting in a DNA fragment.

A “methylation status” refers to the state of methylation at a given site. For example, a site may be either methylated, unmethylated, or in some cases, undetermined.

The “methylation density” of a region can refer to the number of reads at sites within the region showing methylation divided by the total number of reads covering the sites in the region. The sites may have specific characteristics, e.g., being CpG sites. Thus, the “CpG methylation density” of a region can refer to the number of reads showing CpG methylation divided by the total number of reads covering CpG sites in the region (e.g., a particular CpG site, CpG sites within a CpG island, or a larger region). For example, the methylation density for each 100-kb bin in the human genome can be determined from the total number of cytosines not converted after bisulfite treatment (which corresponds to methylated cytosine) at CpG sites as a proportion of all CpG sites covered by sequence reads mapped to the 100-kb region. This analysis can also be performed for other bin sizes, e.g. 500 bp, 5 kb, 10 kb, 50-kb or 1-Mb, etc. A region could be the entire genome or a chromosome or part of a chromosome (e.g. a chromosomal arm). The methylation index of a CpG site is the same as the methylation density for a region when the region only includes that CpG site. The “proportion of methylated cytosines” can refer the number of cytosine sites, “C's”, that are shown to be methylated (for example unconverted after bisulfite conversion) over the total number of analyzed cytosine residues, i.e. including cytosines outside of the CpG context, in the region. The methylation index, methylation density, count of molecules methylated at one or more sites, and proportion of molecules methylated (e.g., cytosines) at one or more sites are examples of “methylation levels.” Apart from bisulfite conversion, other processes known to those skilled in the art can be used to interrogate the methylation status of DNA molecules, including, but not limited to enzymes sensitive to the methylation status (e.g. methylation-sensitive restriction enzymes), methylation binding proteins, single molecule sequencing using a platform sensitive to the methylation status (e.g. nanopore sequencing (Schreiber et al. Proc Natl Acad Sci 2013; 110: 18910-18915) and by single molecule, real-time sequencing (e.g. that from Pacific Biosciences) (Flusberg et al. Nat Methods 2010; 7: 461-465)).

The term “hypomethylation” can refer to a site or set of sites (e.g., a region) that has below a specified threshold for a methylation level, e.g., at or below 50%, 45%, 40%, 35%, 30%, 25%, or 20% for the methylation level. A site in a genome may be considered unmethylated if the methylation level is below a threshold. The term “hypermethylation” can refer to a site or set of sites (e.g., a region) that has above a specified value for a methylation level, e.g., at or above 95%, 90%, 80%, 75%, 70%, 65%, or 60% for the methylation level. A site in a genome may be considered methylated if the methylation level is greater than a threshold. Hypomethylation or hypermethylation can occur for a particular tissue or across a set of tissues.

The terms “hypermethylated” and “hypomethylated” may also refer to the methylation level of a population of DNA molecules as measured by the multiple molecule methylation levels of these molecules. A hypermethylated population of molecules is one in which the multiple molecule methylation level is at or above a threshold which may be defined from application to application, and which may change from application to application. The threshold may be 5%, 10%, 20%, 30%, 40%, 50%, 60%, 70%, 80%, 90%, or 95%. A hypomethylated population of molecules is one in which the multiple molecule methylation level is at or below a threshold which may be defined from application to application. The threshold may be 5%, 10%, 20%, 30%, 40%, 50%, 60%, 70%, 80%, 90%, and 95%. The population may be from a single tissue type of multiple tissue types. The population of molecules may be aligned to one or more selected genomic regions. The selected genomic region(s) may be related to a disease such as a genetic disorder, an imprinting disorder, a metabolic disorder, or a neurological disorder. As examples, the selected genomic region(s) can have a length of great than, equal, or less than 50 nucleotides (nt), 100 nt, 200 nt, 300 nt, 500 nt, 1000 nt, 2 knt, 5 knt, 10 knt, 20 knt, 30 knt, 40 knt, 50 knt, 60 knt, 70 knt, 80 knt, 90 knt, 100 knt, 200 knt, 300 knt, 400 knt, 500 knt, or 1 Mnt.

The term “sequencing depth” refers to the number of times a locus is covered by a sequence read aligned to the locus. The locus could be as small as a nucleotide, or as large as a chromosome arm, or as large as the entire genome.

A sequence read can include an “ending sequence” associated with an end of a fragment. The ending sequence can correspond to the outermost N bases of the fragment, e.g., 1-30 bases at the end of the fragment. If a sequence read corresponds to an entire fragment, then the sequence read can include two ending sequences. When paired-end sequencing provides two sequence reads that correspond to the ends of the fragments, each sequence read can include one ending sequence.

The terms “size profile” and “size distribution” generally relate to the sizes of DNA fragments in a biological sample. A size profile may be a histogram that provides a distribution of an amount of DNA fragments at a variety of sizes. Various statistical parameters (also referred to as size parameters or just parameter) can distinguish one size profile to another. One parameter is the percentage of DNA fragment of a particular size or range of sizes relative to all DNA fragments or relative to DNA fragments of another size or range.

A “calibration sample” can correspond to a biological sample whose fractional concentration of clinically-relevant DNA (e.g., tissue-specific DNA fraction) is known or determined via a calibration method, e.g., using an allele specific to the tissue, such as in transplantation in a pregnant subject whereby an allele present in the donor's genome but absent in the recipient's genome can be used as a marker for the transplanted organ.

A “calibration data point” includes a “calibration value” and a measured or known fractional concentration of the clinically-relevant DNA (e.g., DNA of particular tissue type). The calibration value can be determined from relative frequencies (e.g., an aggregate value) as determined for a calibration sample, for which the fractional concentration of the clinically-relevant DNA is known. The calibration data points may be defined in a variety of ways, e.g., as discrete points or as a calibration function (also called a calibration curve or calibration surface). The calibration function could be derived from additional mathematical transformation of the calibration data points.

The term “classification” as used herein refers to any number(s) or other characters(s) that are associated with a particular property of a sample. For example, a “+” symbol (or the word “positive”) could signify that a sample is classified as having deletions or amplifications. The classification can be binary (e.g., positive or negative) or have more levels of classification (e.g., a scale from 1 to 10 or 0 to 1), including probabilities. Different techniques for determining a classification can be combined to obtain a final classification from the initial or intermediate classification for each of the different techniques, e.g., by majority vote or a requirement that all initial/intermediate classifications are the same (e.g., positive).

The term “parameter” as used herein means a numerical value that characterizes a quantitative data set and/or a numerical relationship between quantitative data sets. For example, a ratio (or function of a ratio) between a first amount of a first nucleic acid sequence and a second amount of a second nucleic acid sequence is a parameter. The parameter can be used to determine any classification described herein, e.g., with respect to fetal, cancer, or transplant analysis. A normalized amount, e.g., a relative frequency, is an example of a parameter.

The terms “cutoff” and “threshold” refer to predetermined numbers used in an operation. For example, a cutoff size (also referred to as a size cutoff) can refer to a size above which fragments are excluded. As another example, a threshold value may be a value above or below which a particular classification applies. Either of these terms can be used in either of these contexts. A cutoff or threshold may be “a reference value” or derived from a reference value that is representative of a particular classification or discriminates between two or more classifications. A cutoff may be predetermined with or without reference to the characteristics of the sample or the subject. For example, cutoffs may be chosen based on the age or sex of the tested subject. A cutoff may be chosen after and based on output of the test data. For example, certain cutoffs may be used when the sequencing of a sample reaches a certain depth. As another example, reference subjects with known classifications of one or more conditions and measured characteristic values (e.g., a methylation level, a statistical size value, or a count) can be used to determine reference levels to discriminate between the different conditions and/or classifications of a condition (e.g., whether the subject has the condition). A reference value can be selected as representative of one classification (e.g., a mean) or a value that is between two clusters of the metrics (e.g., chosen to obtain a desired sensitivity and specificity). As another example, a reference value can be determined based on statistical simulations of samples. Any of these terms can be used in any of these contexts. Such a reference value can be determined in various ways, as will be appreciated by the skilled person. For example, metrics can be determined for two different cohorts of subjects with different known classifications, and a reference value can be selected as representative of one classification (e.g., a mean) or a value that is between two clusters of the metrics (e.g., chosen to obtain a desired sensitivity and specificity). As another example, a reference value can be determined based on statistical simulations of samples. A particular value for a cutoff, threshold, reference, etc. can be determined based on a desired accuracy (e.g., a sensitivity and specificity).

The term “level of cancer” can refer to whether cancer exists (i.e., presence or absence), a stage of a cancer, a size of tumor, whether there is metastasis, the total tumor burden of the body, the cancer's response to treatment, and/or other measure of a severity of a cancer (e.g., recurrence of cancer). The level of cancer may be a number or other indicia, such as symbols, alphabet letters, and colors. The level may be zero. The level of cancer may also include premalignant or precancerous conditions (states). The level of cancer can be used in various ways. For example, screening can check if cancer is present in someone who is not previously known to have cancer. Assessment can investigate someone who has been diagnosed with cancer to monitor the progress of cancer over time, study the effectiveness of therapies or to determine the prognosis. In one embodiment, the prognosis can be expressed as the chance of a patient dying of cancer, or the chance of the cancer progressing after a specific duration or time, or the chance or extent of cancer metastasizing. Detection can mean ‘screening’ or can mean checking if someone, with suggestive features of cancer (e.g., symptoms or other positive tests), has cancer. A level for various types of cancer can be determined, e.g., carcinoma or sarcoma, melanoma, lymphoma, and leukemia, as well as in various tissue of origin, including by way of example: breast, lung, liver, colon, pancreas, stomach, bone, blood, head and neck (e.g., head and neck squamous cell carcinoma), throat, bladder, kidney, prostate, uterine, rectal, bile duct, brain, eye, esophageal, ovarian, oral cavity, Nasopharyngeal, thyroid, urethral, testicular, vaginal, and pituitary.

The abbreviation “bp” refers to base pairs. In some instances, “bp” may be used to denote a length of a DNA fragment, even though the DNA fragment may be single stranded and does not include a base pair. In the context of single-stranded DNA, “bp” may be interpreted as providing the length in nucleotides.

The abbreviation “nt” refers to nucleotides. In some instances, “nt” may be used to denote a length of a single-stranded DNA in a base unit. Also, “nt” may be used to denote the relative positions such as upstream or downstream of the locus being analyzed. For a double-stranded DNA, “nt” may still refer to the length of a single strand rather than the total number of nucleotides in the two strands, unless context clearly dictates otherwise. In some contexts concerning technological conceptualization, data presentation, processing and analysis, “nt” and “bp” may be used interchangeably.

A “machine learning model” (ML model) can refer to a software module configured to be run on one or more processors to provide a classification or numerical value of a property of one or more samples. An ML model can include various parameters (e.g., for coefficients, weights, thresholds, functional properties of function, such as activation functions). As examples, an ML model can include at least 10, 100, 1,000, 5,000, 10,000, 50,000, 100,000, or one million parameters. An ML model can be generated using sample data (e.g., training samples) to make predictions on test data. Various number of training samples can be used, e.g., at least 10, 100, 1,000, 5,000, 10,000, 50,000, 100,000, or at least 200,000 training samples. One example is an unsupervised learning model. Another example type of model is supervised learning that can be used with embodiments of the present disclosure. Example supervised learning models may include different approaches and algorithms including analytical learning, statistical models, artificial neural network (e.g. including convolutional and/or transformer layers), boosting (meta-algorithm), Bayesian statistics, case-based reasoning, decision tree learning, inductive logic programming, Gaussian process regression, genetic programming, group method of data handling, kernel estimators, learning automata, learning classifier systems, minimum message length (decision trees, decision graphs, etc.), multilinear subspace learning, naive Bayes classifier, maximum entropy classifier, conditional random field, nearest neighbor algorithm, probably approximately correct learning (PAC) learning, ripple down rules, a knowledge acquisition methodology, symbolic machine learning algorithms, subsymbolic machine learning algorithms, minimum complexity machines (MCM), random forests, ordinal classification, data pre-processing, handling imbalanced datasets, statistical relational learning, or Proaftn (a multicriteria classification algorithm), or an ensemble of any of these types. The model may include linear regression, logistic regression, deep recurrent neural network (e.g., long short term memory, LSTM), hidden Markov model (HMM), linear discriminant analysis (LDA), k-means clustering, density-based spatial clustering of applications with noise (DBSCAN), random forest algorithm, support vector machine (SVM), or any model described herein. Supervised learning models can be trained in various ways using various cost/loss functions that define the error from the known label (e.g., least squares and absolute difference from known classification) and various optimization techniques, e.g., using backpropagation, steepest descent, conjugate gradient, and Newton and quasi-Newton techniques.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term “about” or “approximately” can mean within an order of magnitude, within 5-fold, and more preferably within 2-fold, of a value. Where particular values are described in the application and claims, unless otherwise stated the term “about” meaning within an acceptable error range for the particular value should be assumed. The term “about” can have the meaning as commonly understood by one of ordinary skill in the art. The term “about” can refer to ±10%. The term “about” can refer to ±5%.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within embodiments of the present disclosure. The upper and lower limits of these smaller ranges may independently be included or excluded in the range (e.g., range can be greater than or less than specified number), and each range where either, neither, or both limits are included in the smaller ranges is also encompassed within the present disclosure, subject to any specifically excluded limit in the stated range. Where the stated range includes one or both of the limits, ranges excluding either or both of those included limits are also included in the present disclosure.

Standard abbreviations may be used, e.g., bp, base pair(s); kb, kilobase(s); pi, picoliter(s); s or sec, second(s); min, minute(s); h or hr, hour(s); aa, amino acid(s); nt, nucleotide(s); and the like.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the embodiments of the present disclosure, some potential and exemplary methods and materials may now be described.

### DETAILED DESCRIPTION

Until recently, the analysis of cell-free DNA molecules involved predominantly short cell-free DNA fragments. Long cell-free DNA molecules may be used to analyze a biological sample. The genomic origin, fragmentomics, and transcriptional correlation of long cell-free DNA can provide information on the molecules, the biological sample, and/or the subject from which the biological is obtained. Analysis using long cell-free DNA fragments, alone or in combination with short cell-free DNA fragments, may improve accuracy or efficiency over analysis using short cell-free DNA fragments alone or other analysis techniques.

To this end, we investigated the origins of long cfDNA molecules from different genomic elements. Remarkably, analysis of plasma cfDNA using long-read sequencing revealed uneven distribution of long molecules from across the genome. Long cfDNA molecules showed overrepresentation in euchromatic regions of the genome, in sharp contrast to short DNA molecules. We observed a stronger relationship between the abundance of long molecules and mRNA gene expression levels, compared with short molecules (Pearson's r=0.71 versus −0.14). Moreover, long and short molecules demonstrated distinct fragmentation patterns surrounding CpG sites. As an example, leveraging the cleavage preferences surrounding CpG sites, the combined ending ratios of long and short molecules differentiated patients with hepatocellular carcinoma (HCC) from non-HCC subjects (AUC=0.874).

We further investigated knockout mice in which selected nuclease genes had been inactivated, in comparison with wild-type mice. The proportion of long molecules originating from transcription start sites were lower in Dffb-deficient mice but higher in Dnase1l3-deficient mice, compared to that of wild-type mice. This work thus provides new insights into the biological properties and potential clinical applications of long cfDNA molecules. Long cfDNA fragments can be defined using various size cutoffs for various techniques described herein, and short cfDNA fragments can be similarly defined using various size cutoffs, which may be different than the size cutoff used to define long cfDNA fragments.

Accordingly, various types of measurements are described, some of which use sizes of cfDNA fragments in the biological sample. Some techniques can detect cancer. For example, a technique can detect cancer using aggregated amounts of cfDNA fragments ending at three or more various positions around different types of CpG sites. The cfDNA fragments can be of a certain size, different size ranges can be analyzed in multiple groups (e.g., long and short as separated groups but with a collective result), or all of the cfDNA fragments analyzed together. Various ratios of amounts, including ratios of a sums of amounts to an amount or sum of amounts, of cfDNA fragments ending at various positions can be used. As another example, machine learning techniques with a feature vector of respective amounts ending at a given position can be used. The use of various types of CpG sites allows more CpG fragments to be used, thereby enabling a smaller sample and/or increased accuracy for a volume of a sample. Similarly, a use of three or more positions also allows more CpG fragments to be used.

Another technique can detect cancer using an amount of cfDNA fragments at a set of open chromatin regions (OCRs). A proportion of cell-free DNA molecules (e.g., long, short, or all) at the set of OCRs can indicate cancer. The proportion can include, but not limited to, various ratios of long to short cfDNA fragments or to cfDNA fragments not in the set of OCR regions. The set of OCRs can correspond to cancer-associated genes, which can be identified as those that are highly expressed (upregulated) in a particular cancer.

Another technique can estimate an expression level of a gene using an abundance of long cfDNA fragments at the gene. The expression level can be determined from discrete classifications (e.g., different categories of differing levels of expression) or as a numerical value. Calibration measurements of gene expression levels using direct measurement techniques (e.g., counting of transcripts from the gene) in calibration samples along with the abundance of long cfDNA fragments in those calibration samples can provide the link that enables the estimation of the expression level indirectly via the abundance of long cfDNA fragments.

Another technique can identify open chromatin regions (OCRs) using an abundance of long cfDNA fragments. As is shown below, the abundance of long cfDNA fragments increases in OCRs. The OCRs can be of various types, e.g., include transcription starting sites (TSSs), DNASE1 hypersensitive sites (DHSs), and CCCTC-binding factor sites (CTCFs).

Another technique can determine a fractional concentration of cell-free DNA of a first tissue type using an abundance of long cfDNA fragments at a set of OCRs. At least some of the set of OCRs can be associated with the first tissue type, e.g., be TSSs specific to the first tissue type. When the first tissue type is tumor tissue, the increase in tumor DNA (e.g., as a tumor grows) can occur across various OCRs or at least in OCRs in which the tumor originates. And the increase in those OCRs can sufficiently influence a larger set of OCRs that include OCRs of other tissue types. Additionally, the tumor tissue can have changed activity in a nuclease (e.g., higher or lower than normal), that affects the size of cfDNA fragments across OCR regions of various tissues. An increase in the fractional concentration can indicate cancer, e.g., as used in the technique that analyzes an amount of cfDNA fragments at a set of open chromatin regions (OCRs).

Another technique can classify an activity of a nuclease using an abundance of long cfDNA fragments. The abundance of long cfDNA fragments can generally be across the genome or within certain regions, such as OCRs, e.g., corresponding to TSSs, DHSs, and CTCFs.

Aspects of implementation can be shared across the different techniques. And similar steps of different techniques can be performed in a similar manner, e.g., measuring sizes of cfDNA molecules.

### I. INTRODUCTION

Fragmentation patterns of cell-free DNA (cfDNA) are demonstrated to be linked to a myriad of biological characteristics, including nuclease activities (Serpas et al. 2019; Han et al. 2020), DNA methylation (Zhou et al. 2022), nucleosome structures (Ivanov et al. 2015; Snyder et al. 2016; Sun et al. 2019), mRNA expression levels (Ulz et al. 2016), and DNA-binding transcription factor activity (Ulz et al. 2019). These characteristics have spurred much research efforts in understanding the underlying biological mechanisms. For example, size analysis of cfDNA molecules reflects characteristic patterns of cfDNA fragmentation. Through short-read sequencing (Illumina), a typical plasma cfDNA fragment size distribution may be depicted with a dominant peak around 166 base pairs (bp), with 10-bp periodicities below 150 bp, suggesting a nucleosomal origin (Lo et al. 2010; Ivanov et al. 2015). Thus, cfDNA is generally believed to be a population of short DNA molecules. Moreover, cfDNA fragments from different tissues may bear information tracing to their tissues of origin. As examples, fetal cfDNA molecules in pregnant women and tumoral cfDNA molecules in patients with cancer are generally shorter than background hematopoietic cell-derived cfDNA (Chan et al. 2004; Lo et al. 2010; Jiang et al. 2015). These findings have catalyzed the use of size information of cfDNA for disease detection (Yu et al. 2014; Jiang et al. 2015; Mouliere et al. 2018).

Single-molecule sequencing technologies, including Pacific Biosciences (PacBio) single-molecule real-time (SMRT) sequencing and Oxford Nanopore Technologies (ONT) sequencing, have opened up the possibility of detecting and characterizing long cfDNA molecules. Recent studies using these platforms have uncovered a population of long cfDNA molecules up to tens of kilobases, in plasma DNA of healthy, pregnant individuals and patients with cancer (Yu et al. 2021; Choy et al. 2022; Yu et al. 2023b, 2023a). Depending on the sequencing platforms used, long molecules account for about a median of 15% and 5% of the total molecules from PacBio and ONT platforms, respectively (Yu et al. 2023b). Hence, single-molecule, long-read sequencing technologies are capable of sequencing a much wider spectrum of cfDNA molecules.

Studies have investigated the quantity, size distribution, methylation patterns and end motifs of cfDNA molecules generated by long-read sequencing (Yu et al. 2021; Choy et al. 2022; Katsman et al. 2022; Lau et al. 2023). As single-molecule sequencing technologies are able to measure methylation patterns (Tse et al. 2021; Lau et al. 2023), one can use methylation patterns to explore the tissues of origin of long cfDNA molecules. Biological properties of long molecules, however, remain largely unexplored. Hence, we investigated whether long and short cfDNA molecules might originate from different genomic elements.

We aggregated cfDNA data generated by SMRT or ONT sequencing to profile the genomic representations and studied the correlation between long cfDNA molecules and gene transcriptional activity. We examined end frequencies of long molecules derived from regulatory regions such as DNASE1 hypersensitive sites (DHSs) and CCCTC-binding factor sites (CTCFs). Moreover, we analyzed cleavage profiles surrounding cytosine-phosphate-guanine sites (CpGs) to investigate long cfDNA fragmentomics in the context of cancer detection. We generated additional cfDNA data from various nuclease-knockout mice using SMRT sequencing to gain biological insights regarding the role of nucleases in the generation of long cfDNA molecules.

Any of the techniques describe below may use sequence reads of cell-free DNA molecules. The sequence reads can be obtained by sequencing the cell-free DNA molecules from the biological sample of the subject. The sequencing can be single-molecule sequencing.

### II. LONG CFDNA ORIGINATE UNEVENLY ACROSS THE HUMAN GENOME

We investigated whether long cfDNA molecules originated evenly across the genome. In this initial example, we considered long cfDNA molecules as those of >500 bp and short cfDNA molecules as those ≤500 bp), but other implementations can use other size cutoffs, which can differ between long and short (e.g., 600 bp for long fragments and 200 for short fragments).

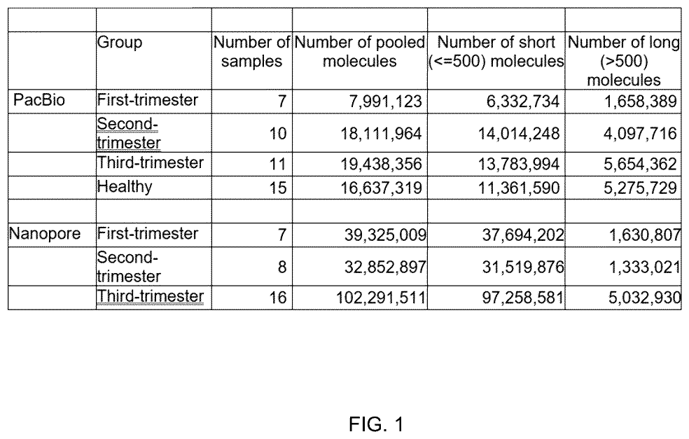

FIG. 1 shows a table of pooled sequenced plasma DNA molecules from previous studies (Yu et al. 2021; Choy et al. 2022; Yu et al. 2023b) using the PacBio and ONT platforms. For both platforms, plasma samples of pregnant subjects at different trimesters were analyzed. And for the PacBio platform, plasma samples from healthy subjects were also analyzed. FIG. 1 shows the number of samples, the number of pooled DNA molecule across the samples of a particular class of subjects (e.g., pregnant at a particular trimester or healthy), the number of short DNA molecules, and the number of long DNA molecules.

We first compared the genomic representation profiles of long (>500 bp) and short (<=500 bp) molecules across 100-kilobase (kb) non-overlapping bins (See section on example implementations for more details). Long and short molecules displayed unevenly in various genomic origins along the whole genome. Interestingly, we observed differential genomic representations between long and short molecules in some regions, both from the SMRT and ONT sequencing data.

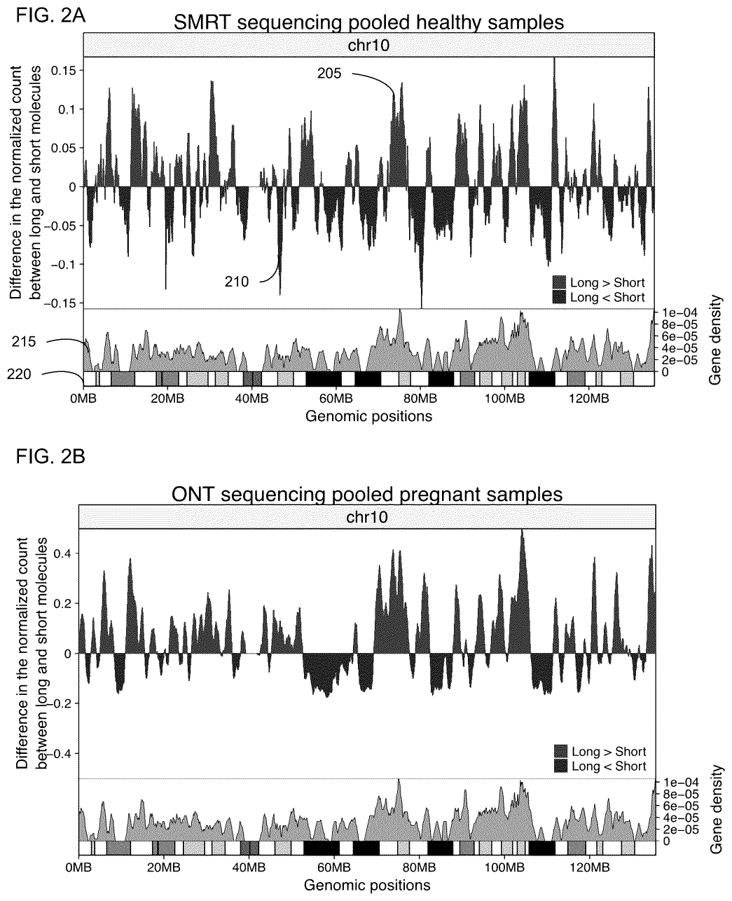

FIG. 2A shows a distribution of long and short molecules from chromosome 10 in human plasma samples as determined using SMRT sequencing for healthy subjects. The vertical axis is a normalized count for a given region, where the normalization divides the difference by the total number of DNA molecules for the region. The horizontal axis shows genomic positions along chromosome 10. Section 215 provides a gene density across the genomic regions. Chromosome 10 shows genomic representation differences between long and short pooled DNA molecules from 15 healthy individuals that were sequenced on the PacBio platform. Overrepresentation 205 and underrepresentation 210 of long cfDNA molecules with respect to short molecules were indicated in color blue and red, respectively. A 100-kb bin size with a 1-Mb smooth window was used for genomic representation. Section 220 shows bands resulting from Giemsa-staining.

FIG. 2B shows a distribution of long and short molecules from chromosome 10 in human plasma samples from 31 first-trimester pregnancies as determined using ONT sequencing. The differential signals in genomic representation were more pronounced in the ONT data. Specifically, we observed that long molecules tended to be overrepresented in regions that overlapped with light bands on Giemsa-stained chromosomes and underrepresented in dark-banded regions shown in ideograms. The light and dark bands typically corresponded to GC-rich euchromatic and AT-rich heterochromatic regions, respectively (Bickmore and Sumner 1989; BAC Resource Consortium et al. 2001). Thus, long molecules were preferentially derived from euchromatic regions compared to short molecules.

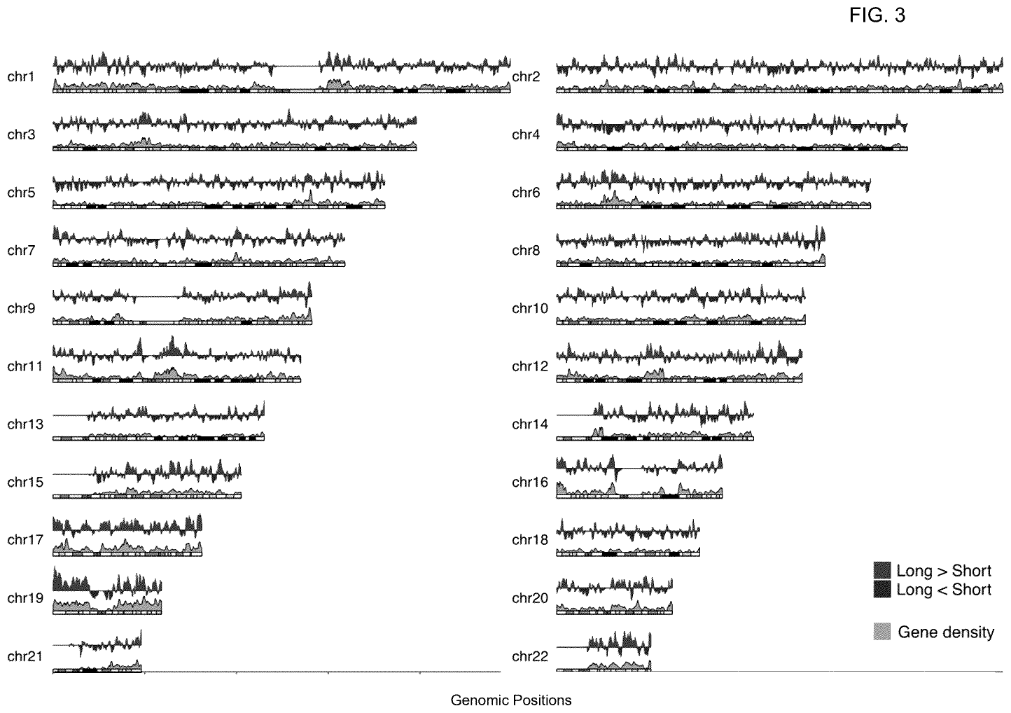

FIG. 3 shows genome-wide representation between long and short pooled DNA molecules from healthy individuals with sequencing on the PacBio platform. The upper track shows genomic representation difference between long and short molecules and the lower track shows the gene density.

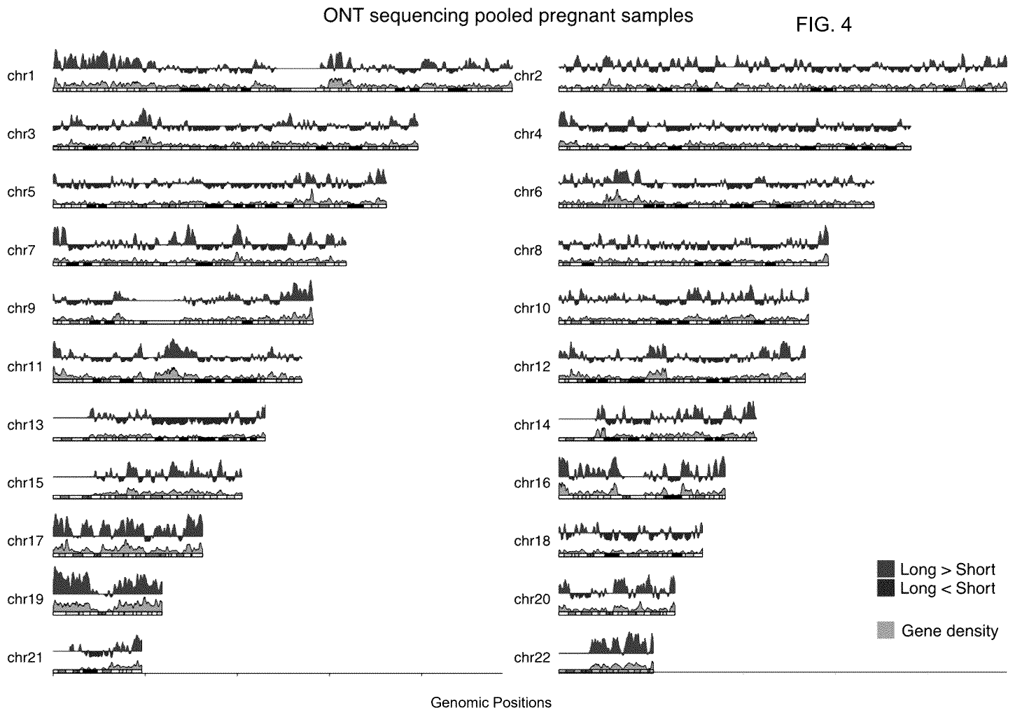

FIG. 4 shows genome-wide representation between long and short pooled DNA molecules from pregnant individuals with sequencing on the ONT platform. The upper track shows genomic representation difference between long and short molecules and the lower track shows the gene density.

The differential representation between long and short molecules derived from euchromatic regions was particularly prominent on chromosome 19, which has high GC-content and considered as the most gene-rich chromosome (Grimwood et al. 2004). As euchromatin generally tends to be gene-rich, we wondered if gene density of a genomic region might positively correlate with the difference between long and short cfDNA molecules from that region. Indeed, the difference was significantly correlated to autosomal gene density (Pearson's r=0.56, P<2.2e−16) and the gene-rich chromosome 19 showed a correlation of 0.7 using pooled healthy samples from SMRT sequencing data. The correlation to gene density was 0.76 (P<2.2e−16) for pooled pregnant samples using ONT sequencing data.

### III. PREDICTING EXPRESSION LEVEL BASED ON LONG/SHORT

Motivated by the observation that long molecules appeared to be enriched in euchromatin that was associated with active transcription, we assessed the potential relationship between abundance of long molecules and transcriptional activity. First, we analyzed the abundance of long and short cfDNA molecules originating from unexpressed and housekeeping genes (see section on example implementations for more details). We also analyzed abundance of long and short cfDNA molecules for genes of different expression levels.

### A. Unexpressed vs Housekeeping

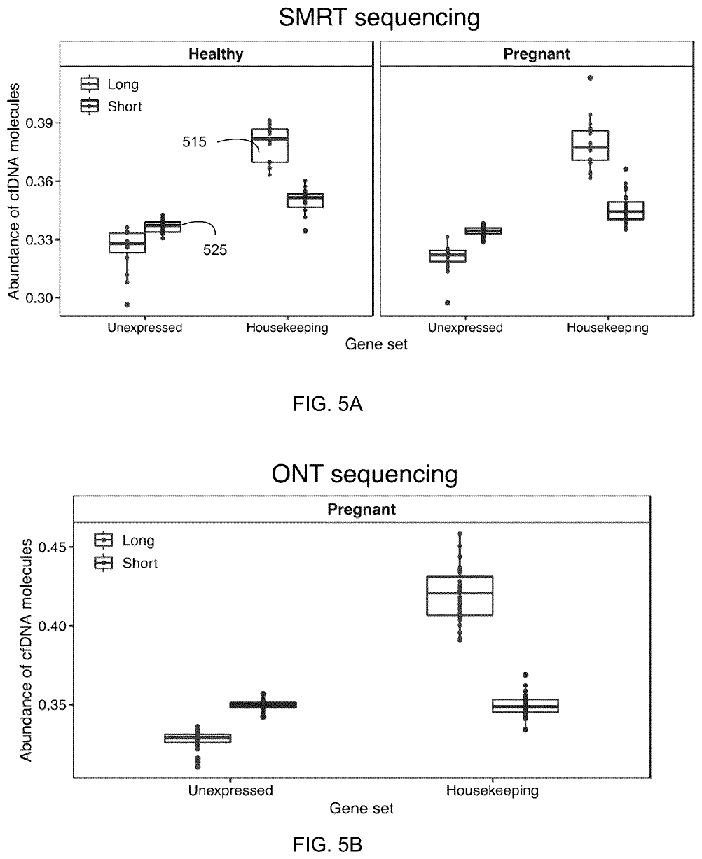

FIG. 5A shows the abundance of SMRT long and short molecules on gene bodies of unexpressed and housekeeping genes for healthy individuals and pregnant subjects. FIG. 5B shows the abundance of ONT long and short molecules on gene bodies of unexpressed genes and housekeeping genes for pregnant subjects. A total of 2,154 unexpressed genes were used, where the unexpressed genes had a mean of less than 1 normalized transcript per million across 54 tissues. A total of 3,510 housekeeping genes were used and were experimentally defined in pubmed.ncbi.nlm.nih.gov/23810203. Long cfDNA 515 are on the left, and short cfDNA 525 are on the right.

The vertical axis shows the abundance of long (>500 bp) and short (≤500 bp) cfDNA molecules at the unexpressed genes and the housekeeping genes. A DNA fragment is counted at a particular gene if the fragment overlaps the gene by one or more bases. The abundance of long or short cfDNA at a set of genes can be determined in various ways, e.g., as the fraction out of all of the cfDNA molecules, fraction of only the cfDNA molecules at the set genes, or a ratio of long/short, short/long, long/(long+short), etc., any of which may be expressed as FPKM. For example, the abundance was expressed as FPKM (fragments per kilobase per million reads), which normalizes read count based on gene length and the total number of mapped reads. For example, for a number of sequenced fragments, F, overlapping to a set of gene of interest (e.g. housekeeping genes) with a total genomic size of L, and a total of mapped sequenced fragments of T, the abundance can be calculated by F/(T*L)*10⁹.

Long cfDNA molecules showed higher abundance over housekeeping genes and lower abundance over unexpressed genes in healthy and pregnant subjects. Housekeeping genes have a higher expression than the unexpressed genes.

### B. Abundances at Genes at Different Expression Levels

We further examined the abundance of cfDNA molecules originating from gene bodies of differentially expressed genes. Specifically, we measured the abundance of long and short cfDNA for five gene sets that have different expression levels. Autosomal protein-coding genes were ranked based on their median expression across tissues (e.g., 79 cell types) and grouped into five sets, from a lowly expressed EXP1 to highly expressed EXP5 set. The five genes sets (EXP1-EXP5) correspond to genes with median normalized transcript per million across tissues in the range of [0, 0.001), [0.001, 1), [1, 8), [8, 30), and [30, 11800), with each containing 4836, 3163, 4187, 4816, and 3088 genes, respectively. The five gene sets EXP1-EXP5 had a median gene expression (in normalized transcript per million) of 0, 0.3, 3.5, 15.5, and 55.7, respectively.

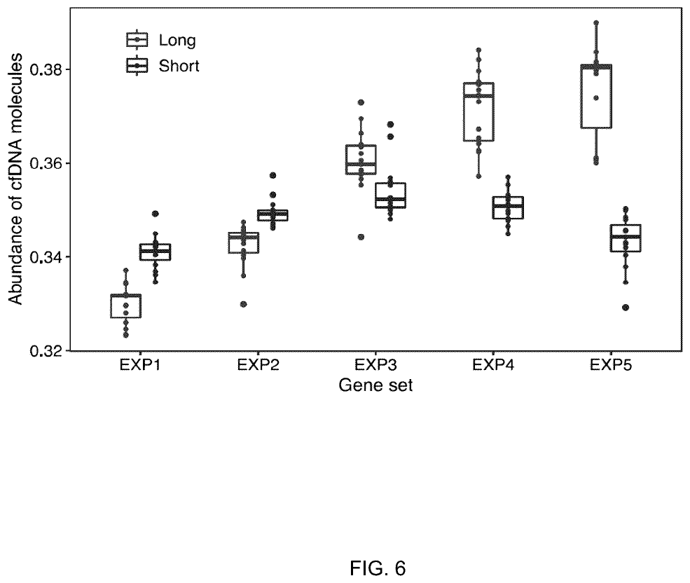

FIG. 6 shows the abundance of SMRT long and short molecules on gene bodies of expression-stratified gene groups for healthy individuals. In line with the enrichment in housekeeping genes, the analysis of healthy samples revealed a stepwise increase of long cfDNA molecules abundance as expression levels increased from EXP1 to EXP5 (P=0.01374, Mann-Kendall trend test; Pearson's r=0.78), while no such trend was found in short molecules (P=0.4032, Mann-Kendall trend test; Pearson's r=−0.28).

We also measured the abundance for of long and short cfDNA for pregnant females.

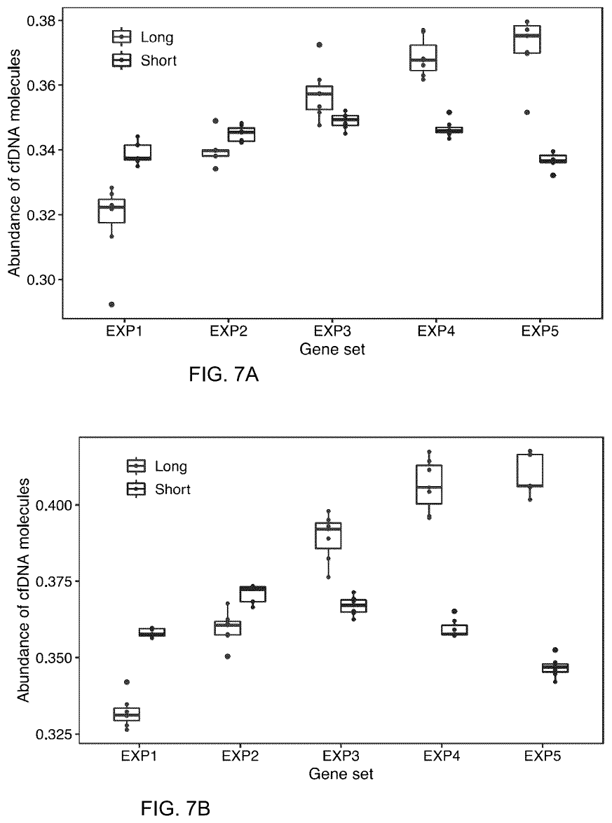

FIG. 7A shows the abundance of long and short molecules on gene bodies of expression-stratified gene groups for the first-trimester individuals sequenced on the PacBio platform. FIG. 7B shows the abundance of long and short molecules on gene bodies of expression-stratified gene groups for the first-trimester individuals sequenced on the ONT platform. We confirmed the observation that long molecule abundance was positively associated with gene expression from SMRT and ONT sequencing data in pregnancies.

We also analyzed the relationship at a higher resolution. A median molecule abundance for long and short cfDNA was determined for different gene sets with various median expression levels across different tissues.

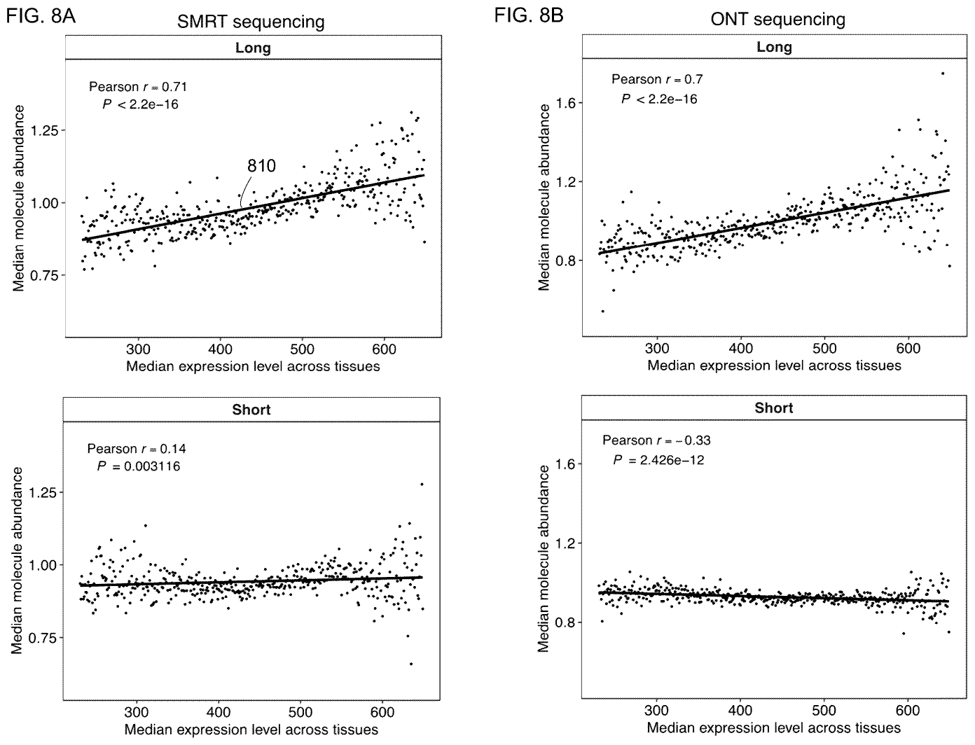

FIG. 8A shows the abundance of SMRT long and short molecules on gene bodies of gene groups having varying median expression levels across tissues for healthy individuals using the PacBio platform. We showed that the median abundances of long molecules were positively correlated (Pearson's r=0.71) with median transformed expression scores across tissues based on 15,556 expressed genes (P<2.2e−16).

In FIG. 8A for the long cfDNA, each of the dots can be considered calibration data points, as a calibration value for the abundance of long cfDNA fragments for a particular gene and gene expression level has been measured. A calibration function 810, which is a line in this example, can be fit to the calibration data points obtained from calibration samples. When a new sample is obtained and a value of a parameter measured (e.g., abundance of long cfDNA fragments), that value can be input to the calibration function to get an estimate of expression level of the gene. Such an input provides an implicit comparison to a calibration value.

As another example, a new measured value for the parameter for a new sample can be compared directly to a calibration value. The abundance of long cfDNA fragments for the new sample can be taken as the expression level of a calibration data points with the closest parameter value to the newly measured one. If more than one calibration data points is identified, a statistical value (e.g., an average or a median) can be determined from the set of closest calibration data points.

FIG. 8B shows the abundance of SMRT long and short molecules on gene bodies of gene groups having varying median expression levels across tissues for healthy individuals using the ONT platform. We showed that the median abundances of long molecules were positively correlated (Pearson's r=0.7) with median transformed expression scores across tissues based on 15,556 expressed genes (P<2.2e−16).

Such positive correlations observed with long molecules were not observed in short molecules (Pearson's r=0.14; P=0.003; FIG. 8A). The conclusion was further confirmed by the ONT data (FIG. 8B).

### C. End Frequency at TSS for Different Gene Expression Levels

In addition to the abundance of molecules, we assessed molecule ends in specific regions. Specifically, end frequencies of cfDNA at transcription starting sites (TSSs) were examined. An end frequency is a rate at which DNA molecules of a group end at a particular position. The end frequency analysis was performed surrounding the TSSs. Transcription starting sites were retrieved using NCBI RefSeq data. The TSSs were grouped according to the gene sets EXP1-EXP5 described above.

The peak or mid-point of the region of interests was regarded as position 0. The end frequencies within +/−2000 bp were normalized with median count of the region, i.e., the 4001 bp for each TSS. The normalized end frequencies were then aggregated each gene set. We applied a 20 bp window to smooth the count (i.e., the value at given position was taken as the average within a 20 bp window around the position). The smoothing can be done before or after normalization.

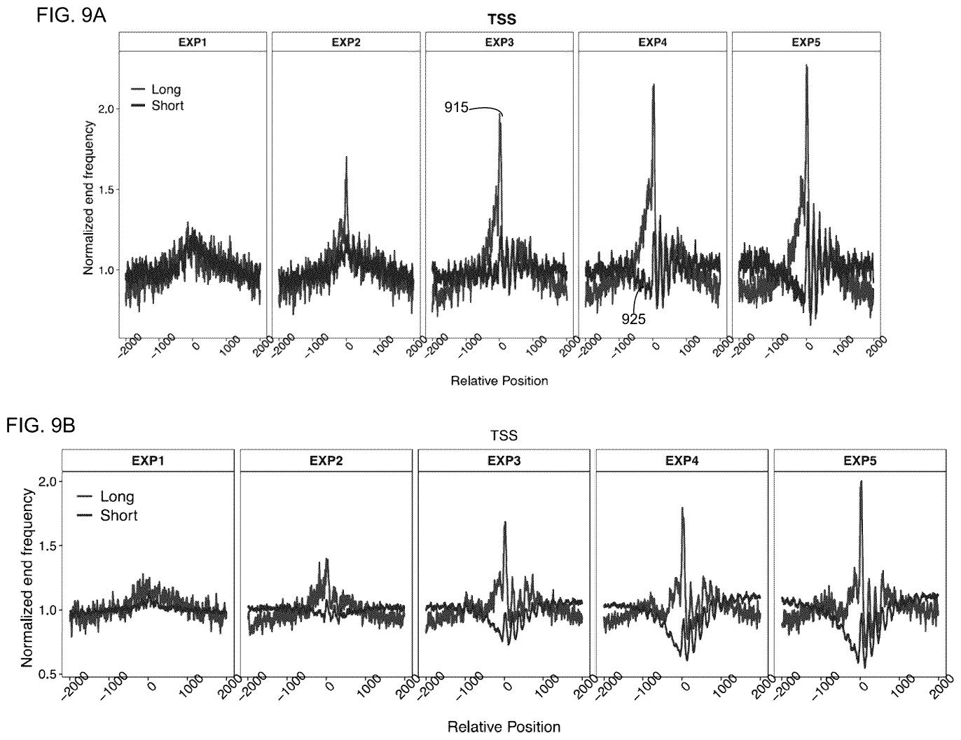

FIG. 9A shows normalized end frequencies of SMRT long and short molecules pooled from plasma of healthy individuals at TSSs of expression-stratified gene groups EXP1 to EXP5, corresponding to low to high expression. Transcription starting positions are denoted as position 0. All transcription starting sites were strand-adjusted so that positive positions are in the direction of transcription. Long cfDNA 915 shows a peak while short cfDNA 925 does not show a well-defined peak.

Interestingly, as the expression level rose, the normalized end frequencies of long molecules gradually increased in the vicinity of TSSs (e.g. 50-bp upstream and downstream of TSSs), rising from 1.01-fold in EXP1 to 1.7-fold in EXP5. Conversely, for short molecules, the normalized end frequencies had a decreasing tendency. The fold change of end frequencies around TSSs was measured using the ratio between long and short cfDNA molecules. Normalized end frequencies from position −50 to 50 were aggregated to compute the ratio.

FIG. 9B shows normalized end frequencies of ONT long and short molecules pooled from plasma of pregnant individuals at TSSs of expression-stratified gene groups EXP1 to EXP5, corresponding to low to high expression. Transcription starting positions are denoted as position 0. All transcription starting sites were strand-adjusted so that positive positions are in the direction of transcription.

This data further indicates that the abundance of long DNA fragments varies proportionality with expression levels.

### D. Different Criteria for Long and Short

We also analyzed the abundance of cfDNA with expression for different definition of long and short DNA molecules. Specifically, we defined short cfDNA fragments to be less than 200 bp and long cfDNA fragments to be greater than 600 bp.

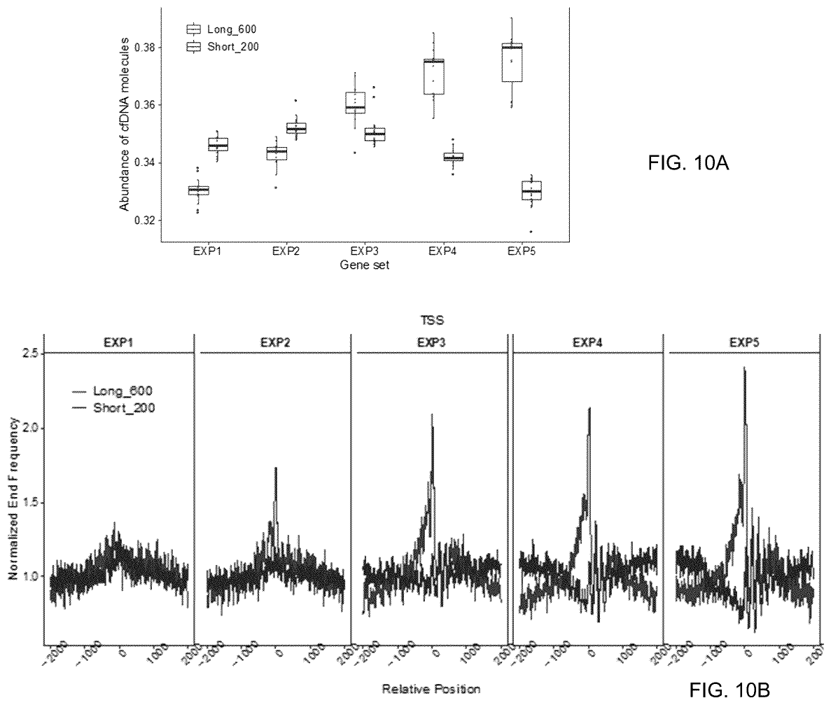

FIG. 10A shows the abundance of long (>600) and short (<200) molecules on gene bodies of expression-stratified gene groups for healthy individuals. FIG. 10B shows normalized end frequencies of long (>600) and short (<200) molecules pooled from plasma of healthy individuals at TSSs of expression-stratified gene groups EXP1 to EXP5, corresponding to low to high expression.

The same behavior can be seen for these different size cutoffs than for the earlier plots. Accordingly, different size cutoffs defining long and short cfDNA fragments can be used. The size cutoff for long cfDNA fragments can be various values as described herein, e.g., at least 400 bp, 500 bp, 600 bp, 700 bp, 800 bp, 900 bp, pr 1 kb bp. The size cutoff for short cfDNA fragments can be different and be various values as described herein, e.g., 150 bp, 200 bp, 250 bp, 300 bp, 350 bp, 400 bp, 450 bp, 500 bp or less.

### E. Method

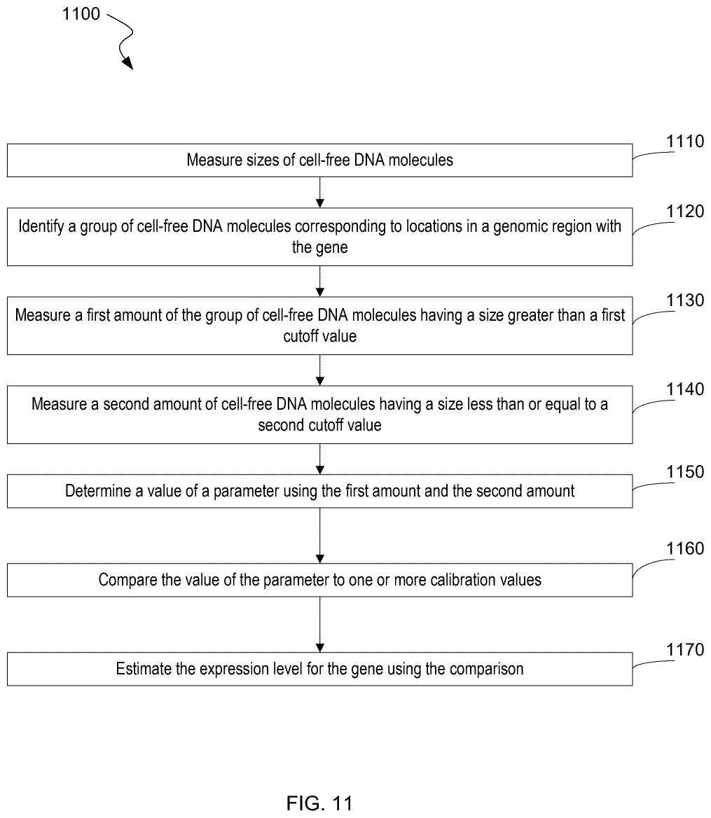

FIG. 11 is a flowchart of an example process 1100 of estimating an expression level of a gene. In some implementations, one or more process blocks of FIG. 11 may be performed by any system described herein. A region with higher expression levels has a greater number of longer cell-free DNA as shown above. The data shows that more long molecules are correlated with a higher expression level. The data also shows that the abundance of short molecules is independent of the expression level. A ratio of long to short can be used. Various such ratios can be used, e.g., long/short, short/long, long/(short+long), etc.

At block 1110, sizes of cell-free DNA molecules are measured. The sizes can be measured in various ways, as can also be for other techniques described herein. For example, measuring the sizes of cell-free DNA molecules in the biological sample can include performing a physical separation of cell-free DNA molecules having different sizes. Examples of such a physical separation include electrophoresis or filtration.

At block 1120, a group of cell-free DNA molecules corresponding to locations in a genomic region within the gene is identified. Such cfDNA molecules can be identified by aligning sequence reads of the cell-free DNA molecules to a reference genome to determine genomic coordinates of ends of the cell-free DNA molecules. The genomic coordinates can be used to determine the cell-free DNA molecules that are located within the genomic region. The sequences reads (e.g., if single molecule sequencing is used) and/or the genomic coordinates can be used to determine the sizes of the cell-free DNA molecules.

The group of cfDNA molecules can be identified in various ways. For example, a cell-free DNA molecule can be considered to be within the gene when an end is located at one or more positions within the genomic region of the gene. The one or more positions can be specific position (e.g., within a window around a center of an OCR region of the gene) or be such that an end can be located anywhere in the genomic region. For instance, the cell-free DNA molecules associated with the gene can be defined as at least 1-nt of a cell-free DNA molecule overlapping with the genomic region. As another example, a cell-free DNA molecule can be considered to be within a gene when at least a specified percentage (e.g., 10%, 20%, 30%, 40% or 50%) of the cell-free DNA molecule overlaps with the genomic region of the gene. For instance, identifying the group of cell-free DNA molecules may include identifying cell-free DNA molecules where each cell-free DNA molecule has at least 50% of its length in the genomic region. The identifying of the group of cell-free DNA molecules can use 3′, 5′, or both ends of cell-free DNA molecules.

At block 1130, a first amount of the group of cell-free DNA molecules having a size greater than a first cutoff value is measured. The cutoff value may be any cutoff value described herein. For example, the first cutoff value can be at least 400 nt.

At block 1140, a second amount of cell-free DNA molecules having a size less than or equal to a second cutoff value is measured. The second cutoff value can be equal to or less than the first cutoff value, although it can be greater than the first cutoff value as well. For example, the second cutoff value can be 500 nt or less.

In various embodiments, a cutoff value used for determining the long and/or short DNA categories could include, but not limited to, 150 nt, 180 nt, 200 nt, 250 nt, 300 nt, 350 nt, 400 nt, 450 nt, 500 nt, 550 nt, 600 nt, 650 nt, 700 nt, 750 nt, 800 nt, 850 nt, 900 nt, 950 nt, 1 knt, 1.1 knt, 1.2 knt, 1.3 knt, 1.4 knt, 1.5 knt, 1.6 knt, 1.7 knt, 1.8 knt, 1.9 knt, 2 knt, 2.5 knt, 3 knt, 4 knt, 5 knt, 6 knt, 7 knt, 8 knt, 9 knt, 10 knt, 15 knt, 20 knt, 30 knt, 40 knt, 50 knt, 60 knt, 70 knt, 80 knt, 90 knt, 100 knt, 200 knt, 300 knt, 400 knt, 500 knt, or 1 Mnt. These cutoff values may apply to any techniques described herein.

At block 1150, a value of a parameter is determined using the first amount and the second amount. The parameter reflects a relative amount between the first amount and the second amount. The parameter may be the first amount normalized by the second amount. The parameter may be the first amount normalized by a total amount of the first amount and the second amount.

At block 1160, the value of the parameter is compared to one or more calibration values. The one or more calibration values may be determined from samples with known expression levels of the gene or of any gene (e.g., a representative set of genes).

At block 1170, the expression level for the gene is estimated using the comparison. The expression level can be a categorical level or numerical. For example, a set of genes can be placed into two or more categories (as in FIG. 6) where different categories have different expression levels. For instance, a calibration value for an abundance of long cfDNA molecules of 0.35 can be identified as corresponding to a level 2, which is for the group of genes labeled as EXP2 in FIG. 6. For numerical, the expression level can be a number, e.g., within a certain precision. Such a number can be obtained with the comparison of the parameter to the one or more calibration values being accomplished by inputting a new measurement of the parameter value to a calibration function. e.g., as shown in FIG. 8A.

Accordingly, comparing the value of the parameter to the calibration value can include inputting the value of the parameter to a calibration function. As another example, a new measured value for the parameter for a new sample can be compared directly to a calibration value. The fractional concentration for the new sample can be taken as the expression level of a calibration data points with the closest parameter value to the newly measured one. If more than one calibration data points is identified, a statistical value (e.g., an average or a median) can be determined from the set of closest calibration data points.

Process 1100 may include additional implementations, such as any single implementation or any combination of implementations described herein and/or in connection with one or more other processes described elsewhere herein.

In embodiments, the gene is expressed by a first tissue, and the expression level is a tissue-specific expression level. In embodiments, process 1800 further includes comparing the expression level to one or more reference values, where the one or more reference values are determined from samples with known fractional concentrations of cell-free DNA molecules from the first tissue. Process 1800 may include estimating the fractional concentration of cell-free DNA molecules from the first tissue using the comparison.

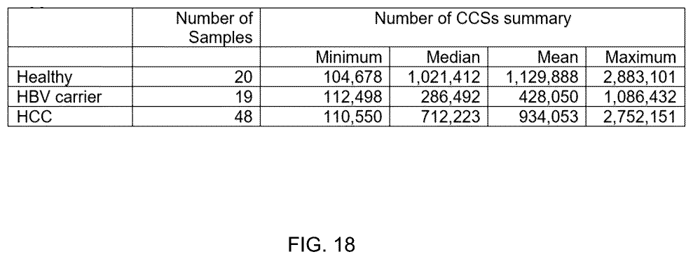

Although FIG. 18 shows example blocks of process 1800, in some implementations, process 1800 may include additional blocks, fewer blocks, different blocks, or differently arranged blocks than those depicted in FIG. 18. Additionally, or alternatively, two or more of the blocks of process 1800 may be performed in parallel.

The gene estimated expression level of the gene can be used in various ways, e.g., to detect a disease or disorder. For example, gene expression can reflect pathophysiological status of multiple tissues in diseases. Gene expression can provide information about a proportion of the cfDNA that is from a particular tissue, e.g., if a gene is uniquely expressed in a particular tissue.

### IV. IDENTIFYING OPEN CHROMATIN REGIONS

Given that TSSs occur in open chromatin regions and long cfDNA fragments increase at TSSs, we further investigated the abundance of long cfDNA fragments in other types of open chromatin regions. Specifically, we analysed end frequencies of cfDNA at regulatory regions that include DNASE1 hypersensitive sites (DHSs) and CCCTC-binding factor sites (CTCFs).

This data is surprising given that it had been shown that cell-free DNA, such as plasma DNA, is generally shorter and less intact, namely of poor intact probability, or poorer integrity, within open chromatin domains, including around transcription start sites, and at locations between nucleosomal cores, such as at the linker positions (Straver et al Prenat Diagn 2016, 36:614-621).

### A. DHS and CTCF

DHSs and CTCFs were identified from publicly available data. Further details are provided in the section for example implementations. The peak or mid-point of the region of interests was regarded as position 0. The end frequencies within +/−2000 bp were aggregated and normalized with median count of the region, as was done for TSSs, but a smoothing window of 10 bp was used.

Fold change of end frequencies around DHSs was measured as follows. End frequencies within a DHS region (a median size of 211 bp) were aggregated. In parallel, end frequencies of the equivalent region (i.e., same size) that are at least a specified distance away (e.g., 3000 bp away upstream and downstream) were aggregated and used as a normalization factor. Fold change of end frequencies around TSSs was measured using ratio between the normalized end frequency for long and short DNA molecules. Normalized end frequencies from position −50 to 50 were aggregated to compute the ratio. Other types of normalization and normalization factors may be used. For example, end frequencies at least 2,500, 4,000, 5,000, 10,000 bp or more can be used. And only upstream of downstream end frequencies can be used.

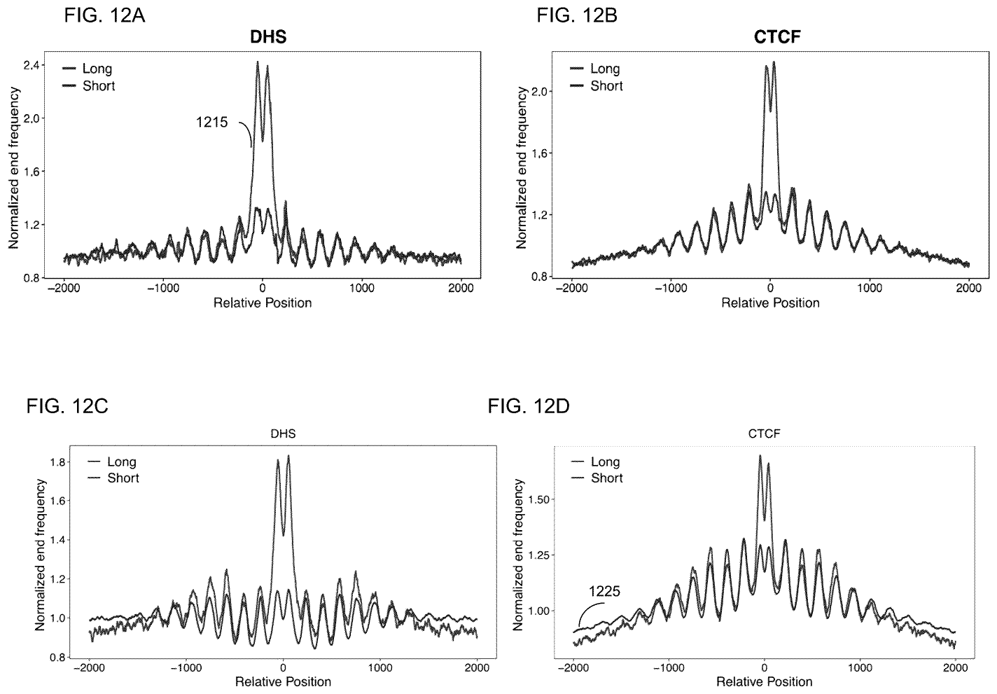

FIG. 12A shows normalized end frequencies of SMRT long and short molecules pooled from plasma of healthy individuals at DHSs. FIG. 12B shows normalized end frequencies of SMRT long and short molecules pooled from plasma of healthy individuals at CTCFs. FIG. 12C shows normalized end frequencies of ONT long and short molecules pooled from plasma of pregnant individuals at DHSs. FIG. 12D shows normalized end frequencies of ONT long and short molecules pooled from plasma of pregnant individuals at CTCF. Long cfDNA 1215 shows a peak while short cfDNA 1225 does not show a well-defined peak.

Long molecules pooled from the plasma of healthy individuals displayed higher end frequencies of cfDNA near the peak of tissue-invariant DHSs (FIG. 12A). We observed that the end frequency of long cfDNA molecules were two times that of short molecules in DHSs. As a confirmation, the pattern was observed in plasma of pregnant women from ONT sequencing (FIG. 12C). At CTCFs, long molecules exhibited higher cfDNA end frequencies as well, in both SMRT and ONT sequencing data (FIG. 12B and FIG. 12D). These results have further highlighted that long molecules were preferentially derived from open chromatin regions and their abundance was significantly correlated with transcriptional activities.

### B. Different Size Cutoffs

We also analyzed the abundance of cfDNA with expression for different definition of long and short DNA molecules. Specifically, we defined short cfDNA fragments to be less than 200 bp and long cfDNA fragments to be greater than 600 bp.

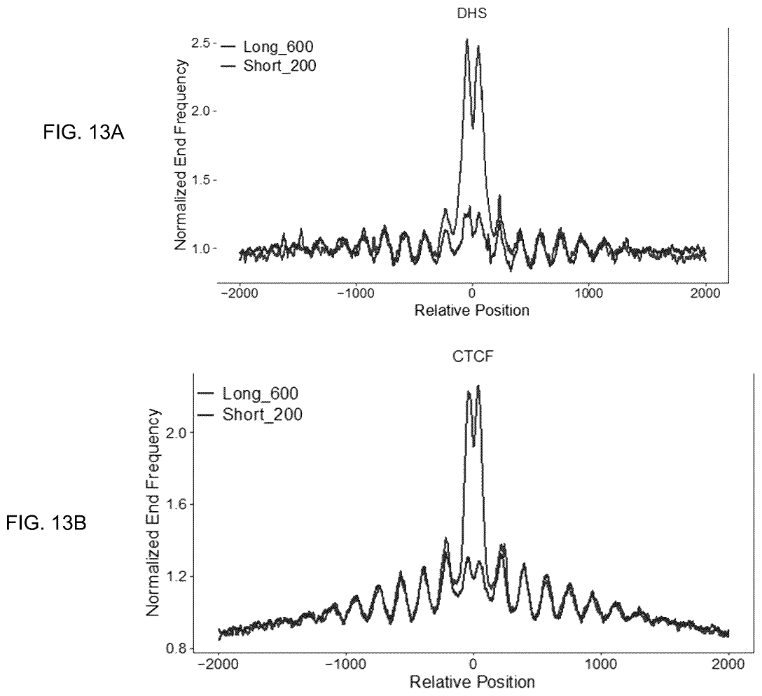

FIG. 13A shows normalized end frequencies of long (>600 bp) and short (<200 bp) molecules pooled from plasma of healthy individuals at DHSs. FIG. 13B shows normalized end frequencies of long (>600 bp) and short (<200 bp) molecules pooled from plasma of healthy individuals at CTCFs.

The same behavior can be seen for these different size cutoffs than for the earlier plots. Accordingly, different size cutoffs defining long and short cfDNA fragments can be used. The size cutoff for long cfDNA fragments can be various values as described herein, e.g., at least 400 bp, 500 bp, 600 bp, 700 bp, 800 bp, 900 bp, pr 1 kb bp. The size cutoff for short cfDNA fragments can be different and be various values as described herein, e.g., 150 bp, 200 bp, 250 bp, 300 bp, 350 bp, 400 bp, 450 bp, 500 bp or less.

### C. Method

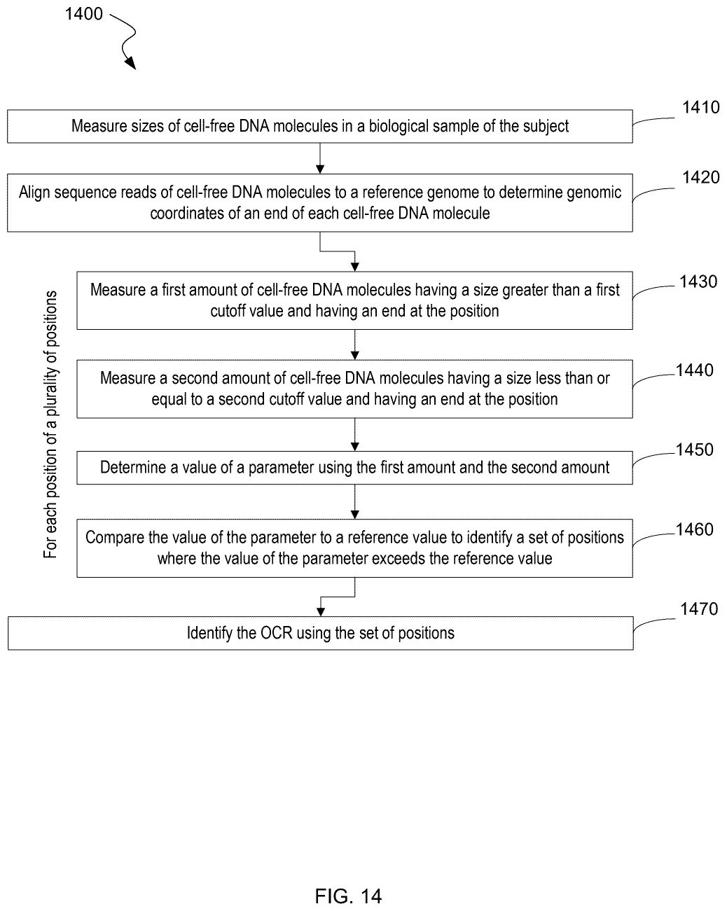

FIG. 14 is a flowchart of an example process 1400 for identifying an open chromatin region (OCR) in a genome of a subject. In some implementations, one or more process blocks of FIG. 14 may be performed by any system described herein. Open chromatin regions (OCRs), including TSS, DHS, and CTCF, are characterized by a high end frequency of long ends. OCRs can be identified, avoiding more laborious techniques, such as ChIP-Seq.

At block 1410, sizes of cell-free DNA molecules in a biological sample of the subject are measured. The sizes can be measured in various ways, as can also be for other techniques described herein. For example, measuring the sizes of cell-free DNA molecules in the biological sample can include performing a physical separation of cell-free DNA molecules having different sizes. Examples of such a physical separation include electrophoresis or filtration.

At block 1420, sequence reads of cell-free DNA molecules are aligned to a reference genome to determine genomic coordinates of an end of each cell-free DNA molecule. The sequences reads (e.g., if single molecule sequencing is used) and/or the genomic coordinates can be used to determine the sizes of the cell-free DNA molecules. Accordingly, measuring the sizes of cell-free DNA molecules can use the sequence reads.

At block 1430, for each position of a plurality of positions in a genomic region, a first amount of cell-free DNA molecules having a size greater than a first cutoff value and having an end at the position is measured. The first cutoff value may be any cutoff value described herein. For example, the first cutoff value can be at least 400 nt.

At block 1440, for each position of a plurality of positions in a genomic region, a second amount of cell-free DNA molecules having a size less than or equal to a second cutoff value and having an end at the position is measured. The second cutoff value can be equal to or less than the first cutoff value, although it can be greater than the first cutoff value as well. For example, the second cutoff value can be 500 nt or less.

At block 1450, a value of a parameter is determined using the first amount and the second amount for each position. The parameter reflects a relative amount between the first amount and the second amount. The parameter may be a ratio of the first amount over the second amount. The parameter may be a ratio of the first amount over a sum of the first amount and the second amount. The parameter may be a difference between the first amount and the second amount.

At block 1460, the value of the parameter is compared to a reference value for each position. A set of positions where the value of the parameter exceeds the reference value is identified. The reference value is a statistical value calculated using at least a portion of the cell-free DNA molecules. The reference value may be a mean or median of all the cell-free DNA molecules, of cell-free DNA molecules that are not outliers, or of cell-free DNA molecules from one or more reference regions. For instance, the reference value can be determined using values of the parameter at sites known to correspond to an OCR, which can be considered a reference region.

The reference value can depend on the parameter used and take various forms. For example, the reference vale can be an end frequency value or a fold change. The fold change can be relative to the median value within a window of neighboring genomic sites. For example, analyzing fragment end frequencies across upstream and downstream 2 kb of a target site of interest, the fold change required by the reference value can be above at least, but not limited to 1.1, 1.2, 1.4, 1.5, 1.6, 1.7, 1.8, 1.9, 2.0, 2.5, 3.0, 3.5, 4.0, 5.0, 10 etc. The value of the parameter being higher than the reference value indicates the target site is within an OCR.

At block 1470, the OCR is identified using the set of positions. In embodiments, identifying the OCR may include identifying a first position of the set of positions with the maximum value of the parameter, and identifying the OCR as a window of positions around the first position. The window may include at least 50, 100, 200, 300, 400, 500, 600, 700, 800, 900, 1,000, 1,500, or 2,000 positions, or may include less than these numbers of positions. The window may or may not be centered at the first position. In some embodiments, identifying the OCR may include identifying the OCR as the set of positions.

In some embodiments, identifying the OCR may include identifying a first position of the set of positions between a second position with the maximum value of the parameter and a third position with a second highest value of the parameter. The first position may have a local minimum value of the parameter. The OCR may be identified as a window of positions around the first position. As examples, the OCR can include a transcription start site (TSS), a DNase1 hypersensitive site (DHS), a CCCTC-binding factor (CTCF) site, or a combination thereof.

Process 1400 may include additional implementations, such as any single implementation or any combination of implementations described below and/or in connection with one or more other processes described elsewhere herein.

### V. FRACTIONAL CONCENTRATION CFDNA OF PARTICULAR TISSUE TYPE

Different tissue types have different open chromatin regions. And given that long DNA is more abundant in open chromatin regions (as evidence by FIG. 10B and FIGS. 12A-13B), we theorized that if the DNA fragments from a particular tissue type increased then the amount of long DNA fragments at an OCR specific to that particular tissue type would increase. Accordingly, an abundance of long DNA fragments could be used to estimate a tissue fraction from the particular tissue type. The particular tissue type could be clinically-relevant DNA, such as fetal, tumor, or transplant.

### A. Ratio of Long DNA to Tissue Fraction

We analyzed 1,882,229 and 5,500,952 long and short cfDNA molecules originating from 177,845 OCRs among 39 patients with HCC. A cfDNA molecule was considered to be at an OCR when at least 50% size of the molecule overlapped with the OCR. Other percentages can be used, such as at least 5%, 10%, 15%, 20%, 25%, 30%, 35%, 40%, 45%, 50%, 55%, 60%, 65%, or 70%. Additionally or alternatively, a number of nucleotides can be used as the criteria, e.g., at least 1, 2, 3, 4, 5, 6, 7, 8, 9, 10, 15, 20, 50, or 100 nt. The OCRs were identified in the following manner. The DHSs identified in pubmed.ncbi.nlm.nih.gov/32728217/ were used. TSSs were retrieved using NCBI RefSeq data. CTCF binding sites from pubmed.ncbi.nlm.nih.gov/22955616/ were used. A window of 2 kb was used for the OCRs.

The ratio of long (e.g. >500 bp) cfDNA fragments to short (e.g. <500 bp) cfDNA fragments was calculated. A normalization can be performed, e.g., relative to a number of fragments in other regions, such as a flanking region. For instance, the ratio can be normalized by the total cfDNA fragments originating from a non-OCR region, which can be upstream and/or downstream e.g., by 1, 2, 3, 4, or 5 kb) of the OCR and be of various sizes, e.g., 0.5 kb, 1 kb, 1.5 kb, 2 kb, 2.5 kb, or 3 kb in size. The present data used regions 3 kb upstream/downstream and that were 2 kb in size, i.e., regions between 3 and 5 kb away from the OCR region. An example formula is: [Long OCR Count/(Long upstream non-OCR count+Long downstream non-OCR count)]/[Short OCR Count/(Short upstream non-OCR count+Short downstream non-OCR count)].

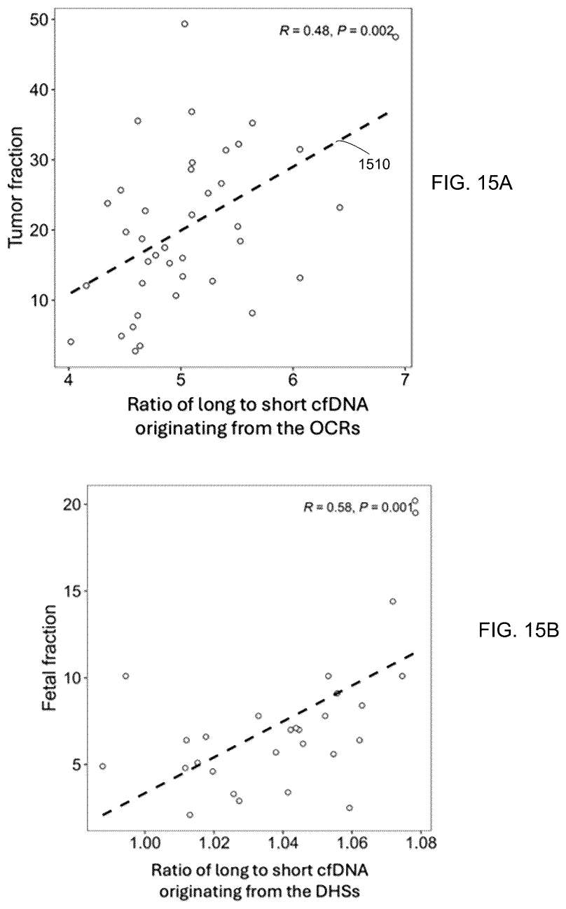

FIG. 15A shows a plot between the ratio of long (>500 bp) to short (<500 bp) cfDNA originating from the OCRs and tumor DNA fraction. The horizontal axis shows the ration of long to short cfDNA from the OCRs. The vertical axis is the tumor DNA fraction. The tumor DNA fraction was deduced by the established method, ichorCNA (Adalsteinsson V. et al. Nature Commun. 2017; 8:1324). We observed that such normalized ratio was correlated with tumor DNA fraction (Pearson's r=0.48, P value=0.002).

In FIG. 15A, each of the dots can be considered calibration data points, as a calibration value for the ration of long to short has been measured and a fractional concentration of the tissue type (tumor tissue in this example) has been measured. A calibration function 1510, which is a line in this example, can be fit to the calibration data points obtained from calibration samples. When a new sample is obtained and a value of a parameter measured (e.g., a ratio of long to short originating from a set of OCRs), that value can be input to the calibration function to get an estimate of the fractional concentration of the first tissue type. Such an input provides an implicit comparison to a calibration value.

As another example, a new measured value for the parameter for a new sample can be compared directly to a calibration value. The fractional concentration for the new sample can be taken as the fractional concentration of a calibration data points with the closest parameter value to the newly measured one. If more than one calibration data points is identified, a statistical value (e.g., an average or a median) can be determined from the set of closest calibration data points.

We also analyzed fetal DNA. We analyzed 1,298,128 and 3,726,747 long and short cfDNA molecules originating from 89,930 placental-specific Dnase I hypersensitivity sites (DHSs) among 28 pregnant subjects. A similar approach was used for the fetal analysis as for the tumor analysis.

FIG. 15B shows a plot between the ratio of long (>500 bp) to short (<500 bp) cfDNA originating from the OCRs and fetal DNA fraction. We observed that the normalized ratio of long to short cfDNA was correlated with fetal DNA fraction (Pearson's r=0.58, P value=0.001). The fetal DNA fraction was deduced by SNP-based approach (Lo et al. Sci Transl Med. 2010; 2:61ra91). These results suggested that the long cfDNA is useful for predicting the clinically-relevant tissue DNA contribution into plasma.

Other types of OCRs can be used, such as TSS and CTCF. Other numbers of OCRs can be used, e.g., at least 100, 200, 500, 1,000, 2,000, 5,000, 10,000, 20,000, 50,000, 100,000, 120,000, 150,000, and 200,000 OCRs. And various numbers of cfDNA fragments can be used, as described herein.

### B. Results for Other Tissues

The ability to detect other tissue types in plasma is illustrated by analyzing end frequencies near tissue-specific DHSs revealed effects of tissue types on long cfDNA fragmentations.

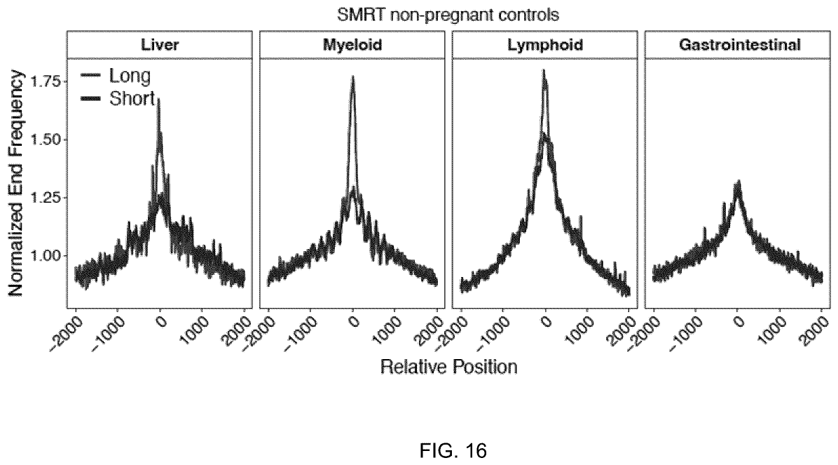

FIG. 16 shows normalized end frequencies of long and short molecules pooled from plasma of healthy individuals at tissue-specific DHSs for liver, myeloid, lymphoid, and gastrointestinal tissues. At liver-specific and myeloid-specific DHSs, long cfDNA in nonpregnant controls tended to have stronger end preferences (1.4-fold) compared with those of short cfDNA. Such a strong preference highlights the relatively high percentage of cfDNA fragments from myeloid cells and a slightly lower percentage from liver cells. Lymphoid cells contribute a smaller amount of DNA, which results in a smaller difference. Gastrointestinal cells contribute a small percentage of cfDNA and thus there is little difference, but if there was gastrointestinal cancer a difference would exist.

### C. Method

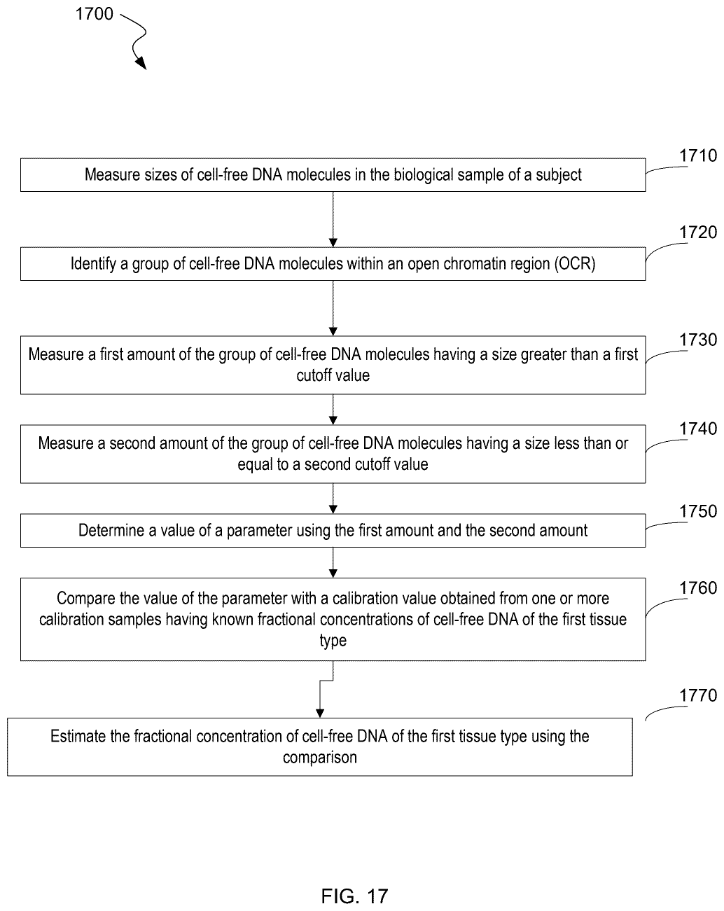

FIG. 17 is a flowchart of an example process 1700 of determining a fractional concentration of cell-free DNA of a first tissue type in a biological sample of a subject. In some implementations, one or more process blocks of FIG. 17 may be performed by any system described herein. An OCR may be associated with certain clinically-relevant DNA of a certain tissue. The amount of fragments in the OCR can then be used to determine a fractional concentration of clinically-relevant DNA (e.g., FIGS. 3A, 3B, and 3C).

At block 1710, sizes of cell-free DNA molecules in the biological sample of a subject are measured. The sizes can be measured in a similar manner as for other techniques. For example, measuring the sizes of cell-free DNA molecules in the biological sample can include performing a physical separation of cell-free DNA molecules having different sizes. Examples of such a physical separation include electrophoresis or filtration.

At block 1720, a group of cell-free DNA molecules within an open chromatin region (OCR) of a set of OCR is identified. Such cfDNA molecules can be identified by aligning sequence reads of the cell-free DNA molecules to a reference genome to determine genomic coordinates of ends of the cell-free DNA molecules. The genomic coordinates can be used to determine the cell-free DNA molecules that are located in the set of OCRs. The sequences reads (e.g., if single molecule sequencing is used) and/or the genomic coordinates can be used to determine the sizes of the cell-free DNA molecules. Various number of OCRs can be used, e.g., at least 10, 20, 50, 100, 1,000, 2,000, 5,000, 10,000, 20,000, 50,000, or 100,000, for this technique and other techniques herein.

The group of cfDNA molecules across the set of OCRs can be identified in various ways. For example, a cell-free DNA molecule can be considered to be within an OCR when an end is located at one or more positions within a window including a center of the OCR. For instance, the cell-free DNA molecules associated with the OCRs can be defined as at least 1-nt of a cell-free DNA molecule overlapping with the OCRs. As another example, a cell-free DNA molecule can be considered to be within an OCR when at least a specified percentage (e.g., 10%, 20%, 30%, 40% or 50%) of the cell-free DNA molecule overlaps with the OCR. For instance, identifying the group of cell-free DNA molecules may include identifying cell-free DNA molecules where each cell-free DNA molecule has at least 50% of its length in the OCR. The identifying of the group of cell-free DNA molecules can use 3′, 5′, or both ends of cell-free DNA molecules.

Various types of OCRs may be used in the set. For example, the set of OCRs may include one or more transcription start sites (TSSs), DNase1 hypersensitive sites (DHSs), or CCCTC-binding factor (CTCF) sites. The group of cell-free DNA molecules may include cell-free DNA molecules having a 5′ end or 3′ end (or either) at the one or more positions central to the open chromatin region.

One or more of the set of OCRs can be associated with the first tissue type. The first tissue type can be tumor cells. As other examples, the first tissue type can be clinically-relevant DNA, e.g., which might increase due to a high level of cell death, such as tumor tissue, fetal tissue, or transplant tissue.

At block 1730, a first amount of the group of cell-free DNA molecules having a size greater than a first cutoff value is measured. The first cutoff value can be various values, e.g., as described herein. For example, the first cutoff value can be at least 400 nt.

At block 1740, a second amount of the group of cell-free DNA molecules having a size less than or equal to a second cutoff value is measured. The second cutoff value can be equal to or less than the first cutoff value, although it can be greater than the first cutoff value as well. For example, the second cutoff value can be 500 nt or less

At block 1750, a value of a parameter is determined using the first amount and the second amount. The parameter can reflect a relative amount between the first amount and the second amount. The parameter can of various types as described above and as described for other techniques. For example, the parameter may be a ratio of the first amount over the second amount. The parameter may be a ratio of the first amount over a sum of the first amount and the second amount. The parameter may be a difference between the first amount and the second amount.

At block 1760, the value of the parameter is compared with a calibration value obtained from one or more calibration samples having known fractional concentrations of cell-free DNA of the first tissue type.

At block 1770, the fractional concentration of cell-free DNA of the first tissue type is estimated using the comparison. The fractional concentration can be expressed as a categorical value or a quantitative value. For example, comparing the value of the parameter to the calibration value can include inputting the value of the parameter to a calibration function, as explained above. As another example, a new measured value for the parameter for a new sample can be compared directly to a calibration value. The fractional concentration for the new sample can be taken as the fractional concentration of a calibration data points with the closest parameter value to the newly measured one. If more than one calibration data points is identified, a statistical value (e.g., an average or a median) can be determined from the set of closest calibration data points.

Process 1700 may include additional implementations, such as any single implementation or any combination of implementations described herein and/or in connection with one or more other processes described elsewhere herein.

Although FIG. 17 shows example blocks of process 1700, in some implementations, process 1700 may include additional blocks, fewer blocks, different blocks, or differently arranged blocks than those depicted in FIG. 17. Additionally, or alternatively, two or more of the blocks of process 1700 may be performed in parallel.

### VI. DETECTING CANCER USING OPEN CHROMATIN REGIONS

As the abundance of long cfDNA molecules in human plasma showed a relationship with gene expression, we explored whether such a signature could be used to distinguish patients with cancer (e.g., hepatocellular carcinoma (HCC)) from subjects without cancer. We also investigated the cfDNA (all and of varying sizes) at open chromatin regions, including regions highly expressed for cancer, e.g., a particular cancer type. Thus, we collected SMRT sequencing data using plasma samples from 20 healthy individuals, 19 hepatitis B virus (HBV) carriers, and 48 patients with HCC.

FIG. 18 shows a table of SMRT sequencing data using plasma samples from 20 healthy individuals, 19 hepatitis B virus (HBV) carriers and 48 patients with hepatocellular carcinoma (HCC). Median values of 1,021,412, 286,492, and 712,223 high-quality circular consensus sequencing reads were obtained for healthy individuals, HBV carriers and patients with HCC, respectively. Long cfDNA molecules accounted for a median of 21.3%, 19.7%, and 23.7% of total molecules in healthy subjects, HBV carriers and patients with HCC, respectively.

### A. Long Short at Genes Upregulated in Cancer

As the number of molecules was relatively low, we examined a group of genes. We analyzed samples from HCC patients and from CRC patients relative to healthy controls.

For CRC, we identified 651 CRC-associated genes that are defined as the upregulation in colon adenocarcinoma (COAD) compared with the adjacent normal tissues, with an adjusted p-value of <0.05 and fold changes of >2 from the database in Zefang Tang, et al., GEPIA: a web server for cancer and normal gene expression profiling and interactive analyses, Nucleic Acids Research, Volume 45, Issue Wi, 3 Jul. 2017, Pages W98-W102, doi.org/10.1093/nar/gkx247. For HCC, we selected 426 genes form public databases.

The p-value can be but not limited to less than 0.01, 0.001, 0.0001, etc. and fold changes can be at least 1.5, 2.5, 3, 4, 5, 6, 7, 8, 9, 10, etc. The adjusted p-value could be calculated by Bonferroni correction, Holm's method, Hochberg's method, Hommel's method, Benjamini-Hochberg method, Benjamini-Yekutieli method, etc. As another example, cancer-associated genes can be defined by a z-score at least above a certain cutoff, such as but not limited to, 1, 2, 3, 4, 5, 6, 7, 8, 9, 10, etc. The z-score metric can be determined by comparing the mean expression in a cancer group of interest (M1) to the mean expression in a reference group (M0) comprising at least one or more normal tissues and/or tumor tissues, normalized by the standard deviation in that reference group (S0) (i.e. z-score=(M1−M0)/S0). Additional criteria can be considered, for example, the minimum expression (RPKM) in the cancer group of interest would be at least but not limited to 0.1, 0.5, 1, 5, 10, 20, 40, 50, etc. The maximum (RPKM) in the reference group would be no more than but not limited to 0.1, 0.5, 1, 5, 10, 20, 30, 40, 50, etc. The difference between the maximum and minimum expressions can be at least but not limited to 1, 2, 3, 4, 5, 10, 20, 30, 40, 50, 100, etc.

Long and short DNA molecules overlapping with the genes were counted and normalized as reads per kilobase per million mapped reads (RPKM): (molecule counts overlapping the set of genes*10³*10⁶/(total read count*genomic size of gene regions)). The relative abundance was then calculated as log 2(long abundance/short abundance).

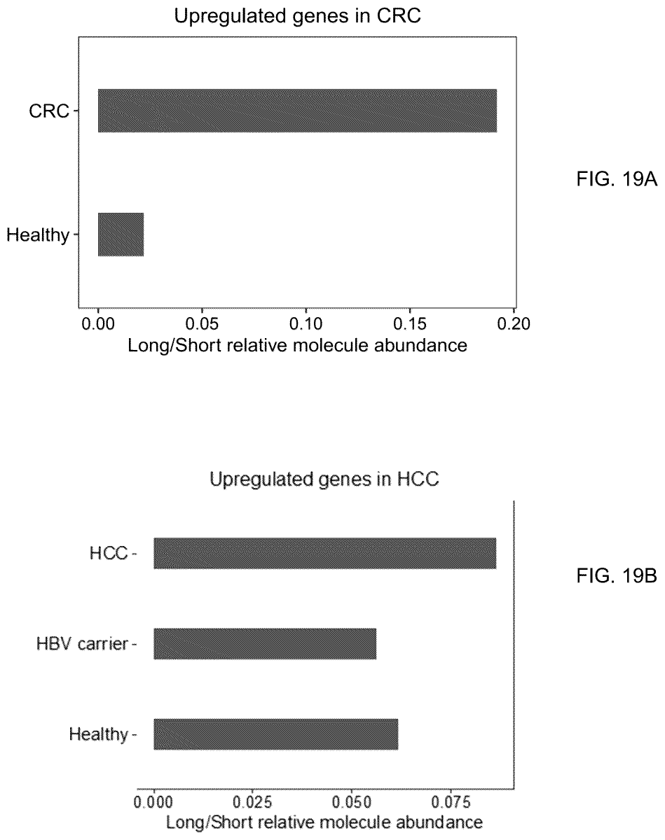

FIG. 19A shows a ratio of an abundance of long cfDNA fragments to short cfDNA fragment at a set of genes that are upregulated in CRC for patients with colon adenocarcinoma (COAD) and healthy controls. The abundances of the 651 CRC-associated genes were calculated for pooled CRC and healthy samples, using long and short molecules respectively. The relative molecule abundance between long and short molecules showed elevation in pooled CRC samples. The ratio is an example of a parameter that can be compared to a cutoff value for discriminating between subjects with CRC and those that are healthy. various ratios can be used, as described herein. Example values for a cutoff include any value between 0.05 and 0.15 for CRC.

FIG. 19B shows a ratio of an abundance of long cfDNA fragments to short cfDNA fragment at a set of genes that are upregulated in HCC for HCC patients, HBV carriers, and healthy controls. We selected 426 genes from public databases that showed higher expression levels in patients with HCC than healthy controls from a public database. The abundances of these selected genes were calculated for pooled HCC, HBV carrier and healthy samples, using long and short molecules respectively. The relative molecule abundance between long and short molecules showed elevation in pooled HCC samples. Example values for a cutoff include any value between 0.05 and 0.15 for CRC.

We also tested accuracy for HCC using more HCC-associated genes and for different number of molecules. HCC-associated genes were identified using curated HCC expression data from The Cancer Genome Atlas (The Cancer Genome Atlas Research Network et al. 2013). Expression values were averaged across 356 HCC tumor tissue samples and ranked by the averaged expression. The top 5000 expressed genes were selected and used as HCC-associated genes. The abundance of long and short molecules over these 5000 genes was calculated. We compared the abundance of long and short molecules over these HCC-associated genes. AUCs were calculated based on the long molecule index.

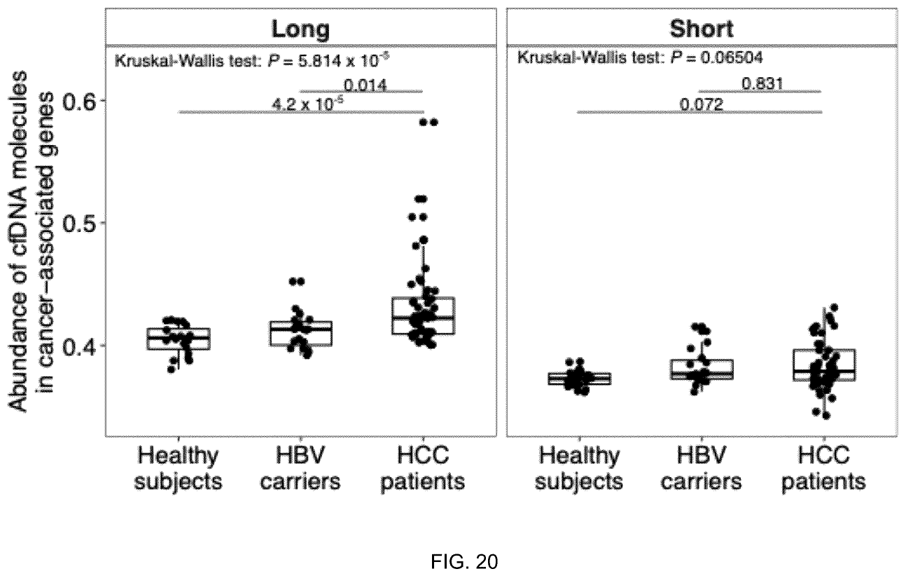

FIG. 20 shows a comparison of the abundance of long and short molecules at 5,000 HCC-associated genes among healthy individuals, HBV carriers and patients with HCC. The Kruskal-Wallis test was used for the P value for differences among groups. Post hoc pairwise Wilcoxon rank sum test P values with Benjamini-Hochberg adjustment are shown above horizontal lines. Long molecules showed significantly higher (P=5.814×10−5, Kruskal-Wallis test) abundance in patients with HCC compared with non-HCC groups, whereas short molecules did not show significant differences (P=0.06504, Kruskal-Wallis test) in this data set.

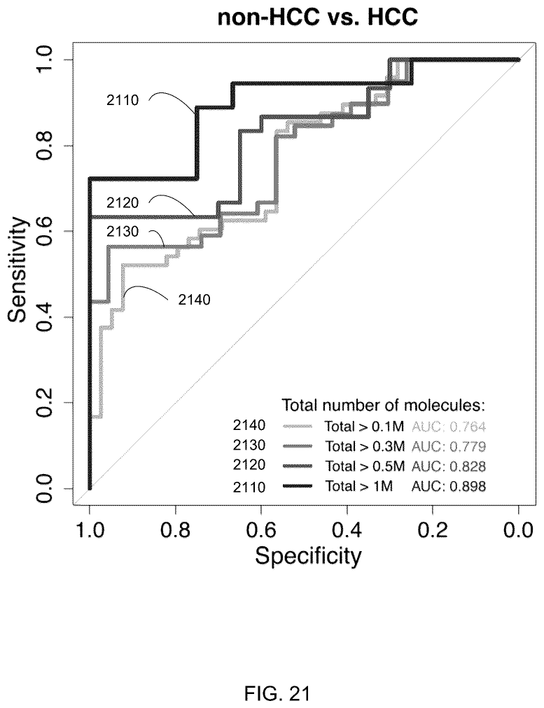

FIG. 21 shows ROCs of long molecule abundance for distinguishing individuals without HCC, including healthy subjects and HBV carriers, and with HCC. Various numbers of molecules were analyzed including curve 2140 with 0.1 million (Total>0.1M), curve 2130 with 0.3 million (Total>0.3M), curve 2120 with 0.5 million (Total>0.5M), and curve 2110 with 1 million (Total>1M) molecules from a sample and used for constructing ROCs. Using 100,000 molecules, a receiver operating characteristic (ROC) curve based on the abundance of long molecules yielded an area under the curve (AUC) of 0.76 to distinguish 39 non-HCC individuals and 48 patients with HCC. When the number of total molecules increased to 1 million for each sample, the AUC improved to 0.9 to distinguish 12 non-HCC individuals and 18 patients with HCC.

### B. Proportion of Long, Short, and all Molecules at OCRs

The plot below shows the proportion of fragment ends in OCR and non-OCR regions. OCRs refer to a specified number (e.g., 500-1000 bp) upstream and downstream of TSSs, DHSs and CTCFs, while non-OCRs refer to regions a specified number of bp (e.g., 3000 bp) upstream and downstream of respective TSSs, DHSs and CTCFs (shown in an illustration). Long proportions were calculated as long molecule ends in OCR divided by total long molecule ends in the sequencing run (number of long molecules*2). Other normalization techniques can be used, e.g., as described herein. Short proportions were calculated as short molecule ends in OCR divided by total short molecule ends. ‘Long+Short’ is the sum of a long proportion and a short proportion. ‘All’ is long and short molecules ends over total molecule ends. Thus, all molecules (long and short) were used to determine the proportion, instead of computing separate ratios and then adding together.

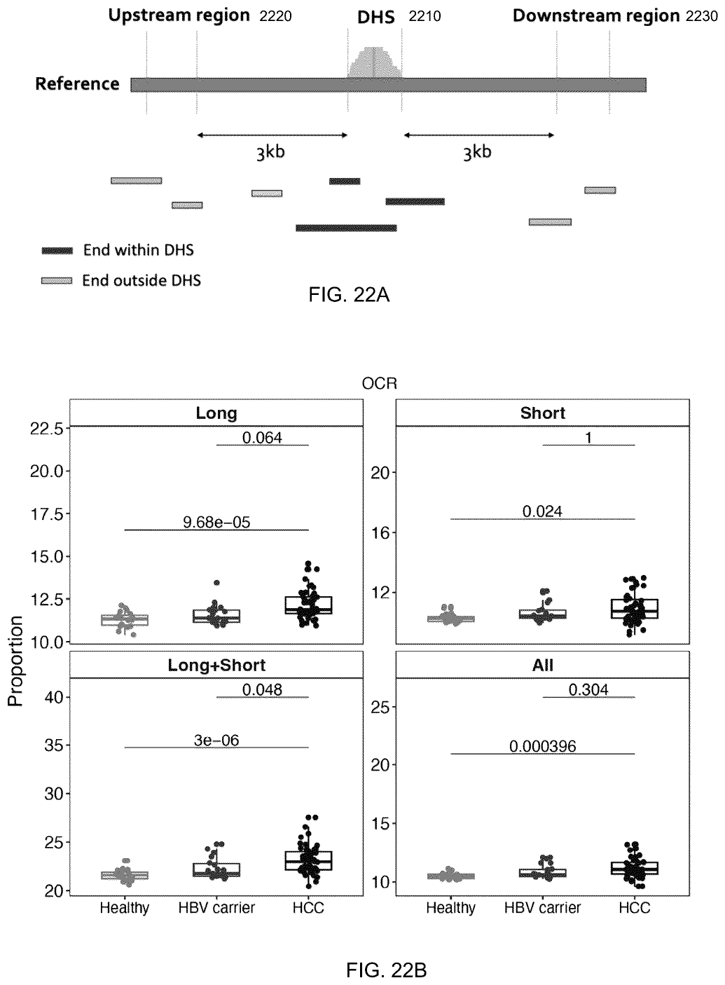

FIG. 22A shows an example way the OCR regions and the non-OCR regions were defined. In this example, the OCR region corresponds to a DHS sites and is labeled as DHS 2210. The OCR region is 2 kb in length, specifically 1000 bp upstream and downstream of the DHS. Other size regions can be used, e.g., 500, 600, 700, 800, or 900 bp, or longer. The non-OCR regions can be the same or different size than the OCR region. The non-OCR region can flank the OCR region, e.g., by being 1 kb, 2 kb, 3 kb, or 4 kb away from a boundary of the OCR. The non-OCR region can be of various sizes, e.g., 0.5 kb, 1 kb, 1.5 kb, 2 kb, 2.5 kb, or 3 kb long. In this example, the non-OCR regions are between −6000 to −4000 and 4000 to 6000 from the DHS.

FIG. 22B shows the proportion of long cfDNA, short cfDNA, long+short cfDNA, and all cfDNA at OCRs. A total of 177,845 OCRs were used. The OCRs were identified in the following manner. The DHSs identified in pubmed.ncbi.nlm.nih.gov/32728217/ were used. TSSs were retrieved using NCBI RefSeq data. CTCF binding sites from pubmed.ncbi.nlm.nih.gov/22955616/ were used. A window of 2 kb was used for the OCRs.

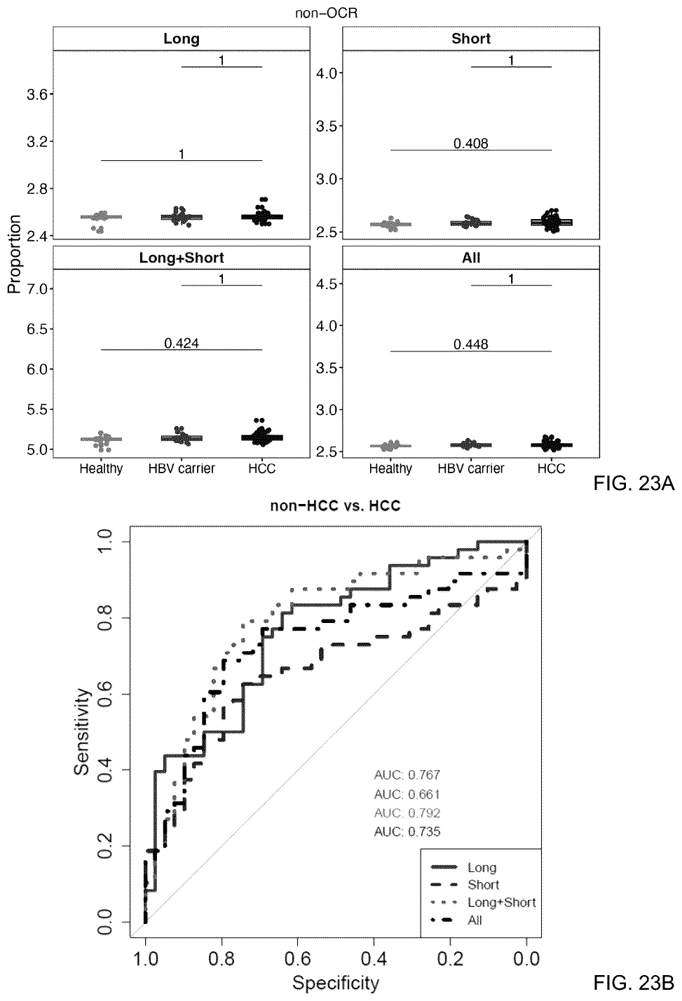

FIG. 23A shows the proportion of long cfDNA, short cfDNA, long+short cfDNA, and all cfDNA at non-OCRs. Twice as many non-OCRs were used as both upstream and downstream regions were used. Counts were averaged between the upstream and downstream regions.

FIG. 23B shows ROC curves using proportions of fragment ends in OCRs. As can be seen, the long+short proportion provided the highest AUC (0.792), with the long proportion (0.767) being second highest, followed by the proportion using all cfDNA fragments (0.735) and the short proportion (0.661).

### C. Method

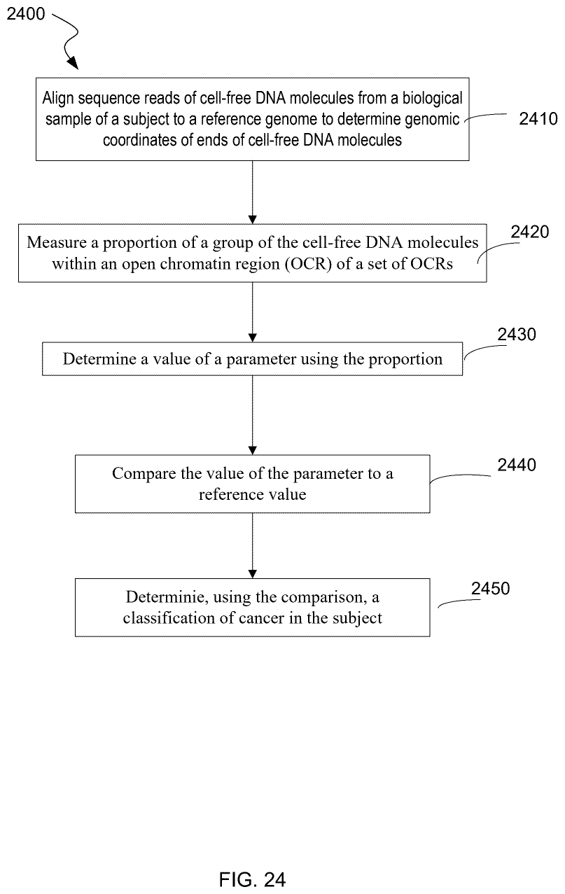

FIG. 24 is a flowchart of a method 2400 for detecting cancer using cfDNA at open chromatin regions in a biological sample of a subject. In various implementations, one or more process blocks of method 2400 (or any process or method described herein) may be performed by measurement systems, computer systems, or any system described herein. As described above, amount of cfDNA fragments at a set of open chromatin regions (OCRs) can be used to determine a classification of cancer (e.g., a level of cancer). A proportion of cell-free DNA molecules (e.g., long, short, or all) at a set of OCRs can indicate cancer. The proportion can include, but not limited to, various ratios of long to short cfDNA fragments or to cfDNA fragments not in the set of OCR regions. The set of OCRs can correspond to cancer-associated genes, which can be identified as those that are highly expressed (upregulated) in a particular cancer. In some embodiments, the biological sample is blood, plasma, or serum. Cell-free DNA can be extracted from such samples using various techniques as will be appreciated by the skilled person.

In various embodiments of any technique described herein, a cutoff value used for determining the long and/or short DNA categories could include, but not limited to, 150 nt, 180 nt, 200 nt, 250 nt, 300 nt, 350 nt, 400 nt, 450 nt, 500 nt, 550 nt, 600 nt, 650 nt, 700 nt, 750 nt, 800 nt, 850 nt, 900 nt, 950 nt, 1 knt, 1.1 knt, 1.2 knt, 1.3 knt, 1.4 knt, 1.5 knt, 1.6 knt, 1.7 knt, 1.8 knt, 1.9 knt, 2 knt, 2.5 knt, 3 knt, 4 knt, 5 knt, 6 knt, 7 knt, 8 knt, 9 knt, 10 knt, 15 knt, 20 knt, 30 knt, 40 knt, 50 knt, 60 knt, 70 knt, 80 knt, 90 knt, 100 knt, 200 knt, 300 knt, 400 knt, 500 knt, or 1 Mnt. These cutoff values may apply to any methods described herein.

At block 2410, sequence reads of cell-free DNA molecules from a biological sample of a subject are aligned to a reference genome to determine genomic coordinates of ends of the cell-free DNA molecules. The cell-free DNA molecules can be long cell-free DNA molecules and have sizes equal to or above a first cutoff value of at least 400 nt. Alternatively or in addition, the cell-free DNA molecules or an additional set of cell-free DNA molecules can be short cell-free DNA molecules, which can have sizes equal to or below a second cutoff value. The second cutoff value can be 500 nt or less. If both sets of cfDNA molecules are used, the second cutoff value can be less than the first cutoff value although it can be greater.

At block 2420, a proportion of a group of the cell-free DNA molecules within an open chromatin region (OCR) of a set of OCRs is measured. The proportion can be measured in various ways. The proportion can be between two values, e.g., long vs short or OCR vs. non-OCR). Accordingly, the group of cell-free DNA molecules can have a size greater than a cutoff value or have a size less than a cutoff value. Both such groups may also be used, e.g., to determine a long+short parameter. Various number of OCRs can be used, e.g., at least 10, 20, 50, 100, 1,000, 2,000, 5,000, 10,000, 20,000, 50,000, or 100,000, for this technique and other techniques herein.

A cell-free DNA molecule can be determined to be within an OCR in various ways. For example, a cell-free DNA molecule can be considered to be within an OCR when an end is located at one or more positions within a window including a center of the OCR. As another example, a cell-free DNA molecule can be considered to be within an OCR when at least a specified percentage (e.g., 10%, 20%, 30%, 40% or 50%) of the cell-free DNA molecule overlaps with the OCR. The identifying of the group of cell-free DNA molecules can use 3′, 5′, or both ends of cell-free DNA molecules.

Each of the one or more OCRs can be associated with the first tissue type. The cancer can be of the first tissue type. In this manner, a type of cancer can be detected. As examples, the set of OCRs can include transcription start sites (TSSs), DNase1 hypersensitive sites (DHSs), CCCTC-binding factor (CTCF) sites, or a combination thereof.

In one example, the proportion can be between long and short cfDNA. A first amount of the group of cell-free DNA molecules having a size greater than a first cutoff value can be measured. A second amount of the group of cell-free DNA molecules having a size equal to or less than a second cutoff value can be measured. The second cutoff value can be equal to or less than the first cutoff value. The proportion can be determined using the first amount and the second amount. The proportion can reflect a relative amount between the first amount and the second amount. In some embodiments, the first cutoff value is at least 400 nt. In some implementations, the second cutoff value is 500 nt or less

As another example, the proportion can be between cfDNA at the set of OCRs and cfDNA at other locations, e.g., the entire reference genome (such as a repeat-masked reference genome) or certain non-OCR regions. The certain non-OCR regions can be those that flank the OCR region, as is described for normalization in other sections and of other techniques described herein. Accordingly, measuring the proportion can comprise measuring a first amount of the group of cell-free DNA molecules and generating the proportion by normalizing the first amount using a second amount of the cell-free DNA molecules that are outside the one or more OCRs. The normalizing can be performed in various ways, e.g., the first amount/second amount or first amount/(first amount+second amount). Thus, normalizing the first amount can use a total amount of the sequence reads aligned to the reference genome.

Such a proportion cfDNA at the set of OCRs and cfDNA at other locations can be done for long and/or short cfDNA molecules. And both such proportion can be used, e.g., for comparing to different reference values (as may be done with a multi-dimensional parameter) or for combining into a single value of the parameter. For example, the group of cell-free DNA molecules can have a size greater than a first cutoff value. Then, another proportion of another group of the cell-free DNA molecules within an OCR of the set of OCRs can be measured. The other group of cell-free DNA molecules can have sizes equal to or less than a second cutoff value. The second cutoff value can be equal to or less than the first cutoff value. The value of the parameter can be determined using the proportion and the other proportion, e.g., a sum or a ratio of the two proportions can be used.

At block 2430, a value of a parameter is determined using the proportion. The value of the parameter can be the proportion. As described above, when two proportions are determined for long or short cfDNA molecules, a sum or a ratio of the two proportions can be used as the value of the parameter. As additional example, the parameter may be determined as a function of the proportion.

At block 2440, the value of the parameter is compared to a reference value. The reference value can be determined using training samples from subjects having known classifications of cancer, e.g., positive or negative for cancer. The training samples that are negative for cancer can include healthy subjects and/or subjects with other diseases that are not cancer, e.g., a viral infection such as HBV.

At block 2450, a classification of cancer in the subject is determined using the comparison. The classification can be a level of the cancer and can include a type of the cancer.

### VII. DETECTING CANCER USING CLEAVAGE PROFILE ACROSS CPG SITES

Studies have demonstrated that the cfDNA cleavage preferences occurred at methylated CpG sites (Han et al. 2021; Zhou et al. 2022). Beyond the fragmentation around regulatory regions, we investigated whether fragmentation of long molecules at CpGs would be different from those of short molecules and whether there was a cleavage pattern for cancer across CpG sites. We analyzed the cleavage patterns at commonly methylated and unmethylated CpGs in pooled healthy samples (See section on example implementation for additional details).

The data specified from FIG. 18 was also analyzed to explore whether the cleavage profile around CpGs for short, long, and all molecules would be associated with diseases. We evaluated cleavage patterns related to all autosomal CpGs and lower numbers of CpGs.

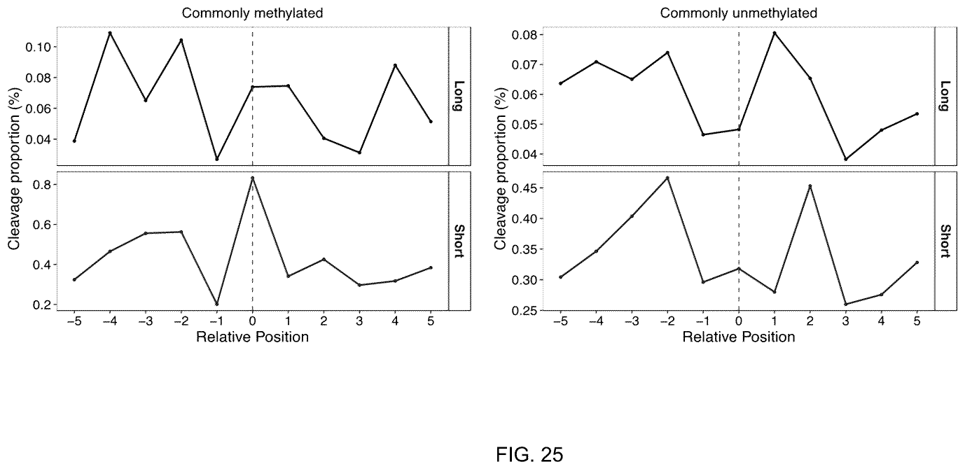

FIG. 25 shows cleavage profiles of pooled PacBio molecules from healthy subjects at commonly methylated (left panel) and commonly unmethylated (right panel) CpGs for long and short cfDNA fragments. A cleavage window of 11 bases is shown. Position 0 and 1 indicate cytosine and guanine, respectively. The vertical axis is a proportion of all long or short DNA fragments ending at a given position. Various normalization techniques can be used. In the example shown, a total number of long or short DNA fragments within the window was used to normalize the cleavage proportion for each window around a CpG site. When data over multiple CpG sites is shown collectively, an average or median can be determined for each position. Other normalization techniques may be used. For example, a normalization for long fragments can use a number of all DNA fragments that are long, and similar for short fragments.

Long cfDNA fragments were defined to be greater than 500 bp, and short cfDNA fragments were defined to be less than or equal to 500 bp. Other size cutoffs can be used as described herein. When size is not being used, the normalization can be similar but for all cfDNA fragments.

The cleavage of short molecules confirmed earlier findings that nuclease-mediated preferential cleavage occurs at methylated CpGs in healthy subjects analyzed by Illumina sequencing (Zhou et al. 2022). Specifically, the cfDNA cleavage was relatively enriched at the cytosine of methylated CpGs (position 0), followed by the rapid decrease at the nucleotide 1 position immediately preceding methylated CpGs.

Interestingly, we found that the cleavage of long molecules did not show such a pattern at methylated cytosine but exhibited the preferred cutting at positions several nucleotides away from the methylated CpGs (e.g., positions −4, −2, and 4).

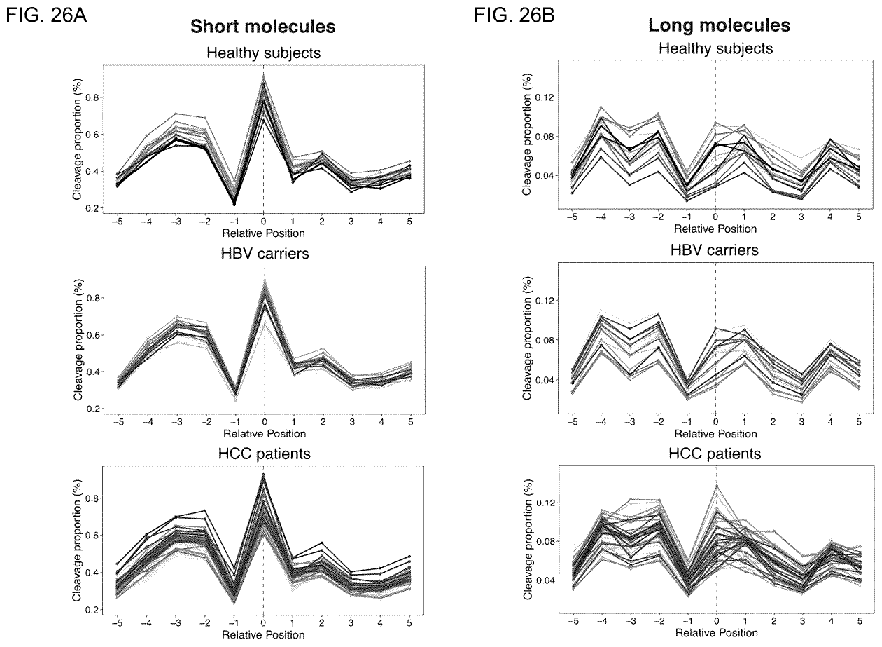

FIG. 26A shows cleavage profiles surrounding all autosomal CpGs for short cfDNA molecules for healthy, HBV, and HCC subjects. Each line represents one sample. A cleavage window of 11 bases is shown. Position 0 and 1 indicate cytosine and guanine, respectively. FIG. 26B shows cleavage profiles surrounding all autosomal CpGs for long cfDNA molecules for healthy, HBV, and HCC subjects.

As the human genome is highly methylated, with 70%-80% of CpGs being methylated (Ziller et al. 2013), the cleavage profiles of short molecules showed cutting preference at position 0 cytosine, similar to the cleavage profile from FIG. 25. The cleavages of long molecules, however, consistently showed a higher cleavage than the following nucleotide at positions −4, −2, 1 and 4 in healthy subjects and HBV carriers. But for HCC patients position 0 had a relatively higher cleavage compared to the healthy subjects and the HBV carriers.

### A. CGN/NCG Ratio for Long and Short DNA Molecules

We investigated a ratio of cfDNA molecules ending at position 0 (CGN) and position −1 (NCG) among healthy, HBV, and HCC subjects for long and short cfDNA fragments. The ratio of 5′ CGN to NCG end motifs (CGN/NCG motif ratio) has been shown to inform methylation level and can be used to distinguish patients with HCC from non-HCC individuals using Illumina sequencing (Zhou et al. 2022).

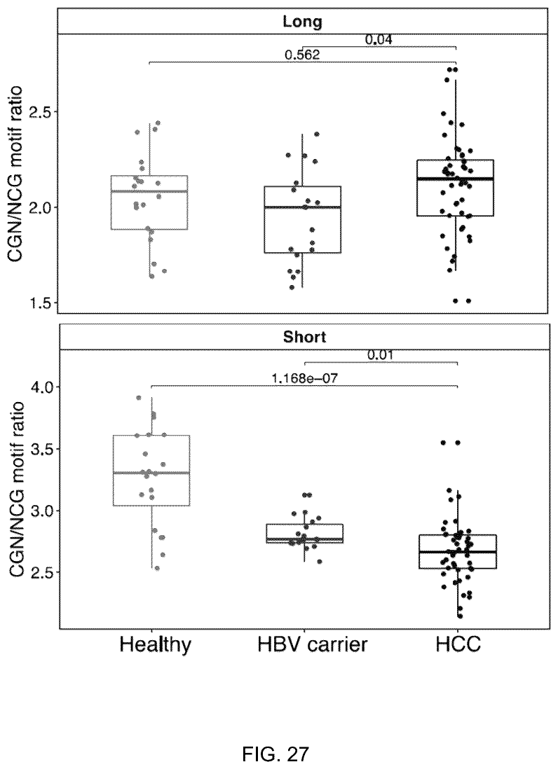

FIG. 27 shows a box plot of CGN/NCG motif ratios for long and short molecules for healthy, HBV, and HCC subjects. In this SMRT sequencing dataset, patients with HCC showed overall lower CGN/NCG motif ratios than the healthy subjects (P=1.168e−07) for short molecules. On the contrary, for long molecules, CGN/NCG motif ratios of patients with HCC showed an upward trend in comparison with healthy subjects but were not statistically significant (P=0.562) different. The separation was much better for short cfDNA fragments than for long cfDNA fragments.

### B. Ending Ratio

We also investigated a ratio of amounts aggregated at multiple positions around a set of CpG sites. We initially aggregated over all 26,752,699 autosome CpGs. Our initial investigation was for aggregation of positions −4, −2, 1, and 4 corresponding to a first aggregated amount. A second aggregated mount was determined for position −1. Both amounts were aggregated over all CpG sites. The aggregation can be done in various ways, e.g., a sum of the raw counts of cfDNA fragments ending at those positions or a sum of individual normalized amounts. For instance, a normalized value at each position can be determined using the raw count ending at that position divided by all fragments within the window around the CpG site. The numerator or the denominator of a ratio (or both) can be a result of an aggregation over multiple positions around the CpG sites. Any of these ratios is referred to as an ending ratio or an aggregated cleavage ratio.

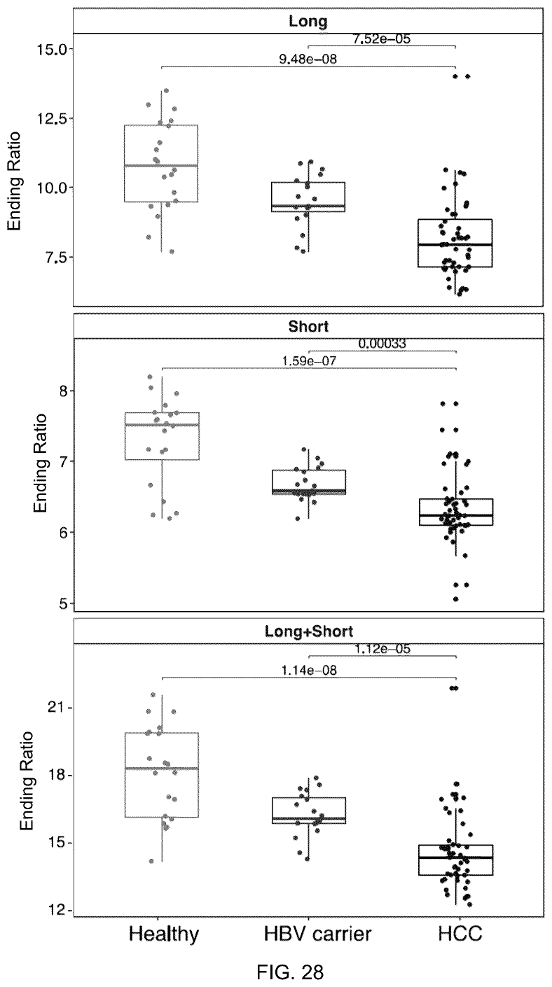

FIG. 28 shows a box plot of ending ratios between aggregating positions −4, −2, 1, and 4 and position −1 for long, short, and long+short molecules. Using this ending ratio, long and short molecules displayed significant differences between patients with HCC and non-HCC subjects for both long and short molecules, as well as a sum of the ending ratios for long and short, referred to as long+short.

The ending ratio for long cfDNA fragments corresponds to a total amount of long cfDNA fragments ending at positions −4, −2, 1, and 4 divided by the number of long cfDNA fragments at position −1. The ending ratio for short cfDNA fragments corresponds to a total amount of short cfDNA fragments ending at positions −4, −2, 1, and 4 divided by the number of short cfDNA fragments at position −1. The ending ratio for long+short is the sum of the long ending ratio and the short ending ratio.

When compared to the separation in FIG. 27, the use of the ending ratio defined for FIG. 28 provides an increased separation. Thus, the use of ending ratios involving three or more positions can provide an incased accuracy over CGN/NCG ratios.

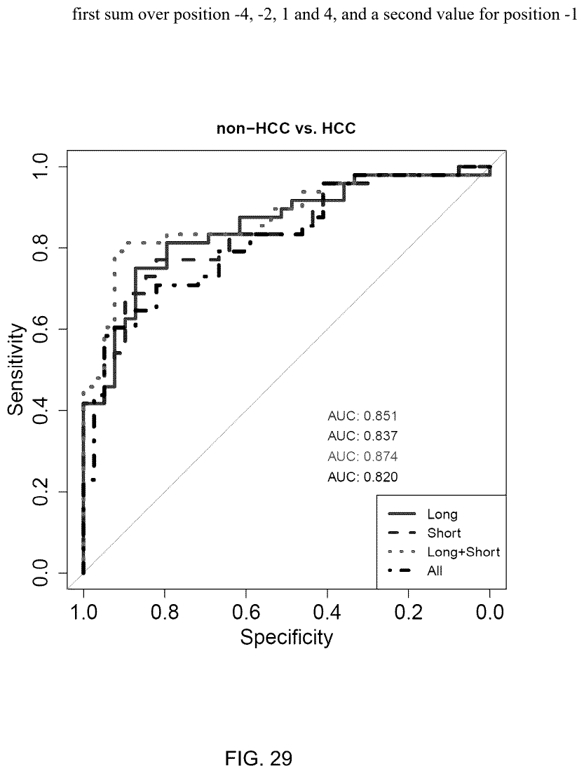

FIG. 29 shows Receiver Operating Characteristics (ROCs) curves for distinguishing patients with HCC from non-HCC subjects for short, long, and long+short cfDNA fragments using an ending ratio of aggregating positions −4, −2, 1, and 4 and position −1. We achieved an area under the curve (AUC) of 0.851 to distinguish 39 non-HCC individuals and 48 patients with HCC using long molecules only. An AUC of 0.837 was achieved for short molecules. Combining the ratio of long and short molecules, the AUC increased to 0.874.

Accordingly, a good separation was achieved using a combined analysis of at least three positions of cleavage around a set of CpG sites. Since all CpG sites were used, a larger amount of cfDNA fragments could be used than if only a certain type of CpG sites were used. In this manner, a sample size could be smaller and/or a greater statistical power and accuracy could be achieved.

### C. Machine Learning

Instead of aggregating a numerator and/or a denominator, we also investigated using machine learning models that perform the aggregation based on an input of the individual amounts at different positions. This example uses positions −5 to +5 for the window around the CpG sites, but other examples can use individual, disjoint values (i.e., not continuous range) or larger or smaller windows. A SVM model was used, but other models can also be used.

We trained a support vector machine model using the cleavage profile that ranges from position −5 to 5. We used leave-one-out cross-validation to assess the performance in cancer detection. A probability estimate was yielded for each sample from the cross-validation procedure. The model training was performed on cleavage profiles of long molecules and short molecules separately.

Additionally, we have also trained a model that concatenated both long and short cleavage profiles as input. Thus, ‘Concatenate’ refers to have positions −5 to 5 from long molecules and positions −5 to 5 from short molecules as input features (22 features); ‘All’ refers to pool long and short molecules together and then compute ratios for positions −5 to 5 (11 features).

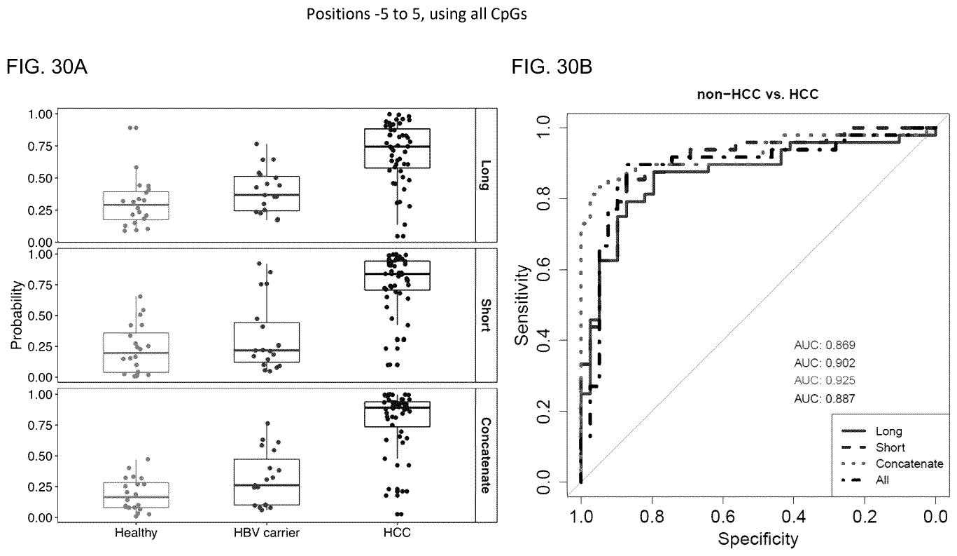

FIG. 30A shows a plot of probabilities for discriminating between healthy, HBV, and HCC subjects for long, short, and concatenation of both using an support vector machine (SVM) model for a window of −5 to +5 around all CpG sites. FIG. 30A shows comparisons of probability scores fitted by leave-one-out cross-validation on the dataset. “Concatenate” refers to training a SVM model using both long and short cleavage profiles. The estimated probability scores showed separations between patients with HCC and non-HCC subjects.

FIG. 30B shows areas under the curve for distinguishing patients with HCC from non-HCC subjects using probabilities scores in FIG. 30A. Using the two cleavage profiles (i.e., concatenating) for long and short cfDNA fragments provided the best AUC of 0.925. The short cfDNA fragments provided better AUC (0.902) than the long cfDNA fragments (0.869). Additionally, FIG. 30B shows results for using one cleavage profile for all cfDNA fragments. The AUC (0.887) for all fragments is between the results for the long and the short cfDNA fragments.

### D. Different Positions

We further investigated using different positions within the window around the CpG sites. The data in this section still uses all 26,752,699 autosome CpGs. In a later section, we investigated using fewer CpG sites.

### 1. Machine Learning

We trained an SVM model in a similar manner but using cleavage profiles of fewer positions, e.g., that ranges from position −4 to 4. The model training was performed on cleavage profiles of long molecules and short molecules separately. Additionally, we have also trained a model that concatenated both long and short cleavage profiles as input and a model using all molecules.

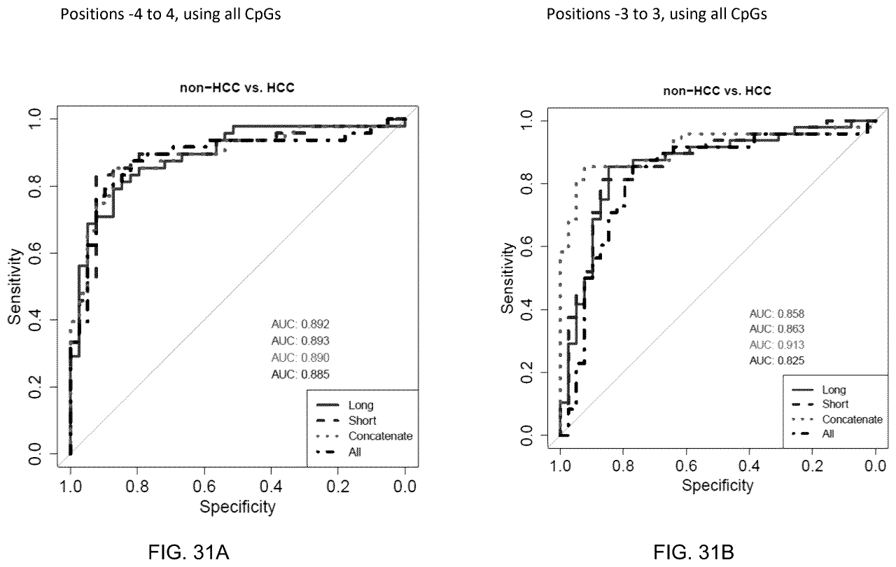

FIG. 31A shows ROC curves for long, short, concatenated, and all molecules using positions −4 to 4 aggregated across all CpG sites. Interestingly, the AUC (0.892) for long cfDNA was better for the −4 to 4 range than for −5 to 5 range. The AUC (0.893) for short cfDNA was lower for the −4 to 4 range, and the concatenated AUC (0.890) was lower as well. The use of all cfDNA was effectively the same. All AUC values for −4 to 4 were similar to each other.

FIG. 31B shows ROC curves for long, short, concatenated, and all molecules using positions −3 to 3 aggregated across all CpG sites. The concatenated AUC (0.913) increased relative to using the −4 to 4 range. Other AUC values were the same.

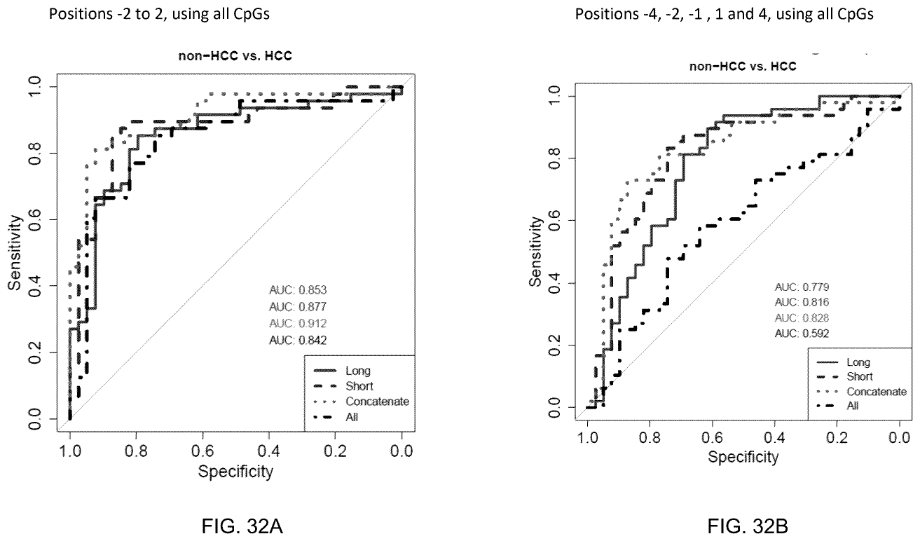

FIG. 32A shows ROC curves for long, short, concatenated, and all molecules using positions −2 to 2 aggregated across all CpG sites. The concatenated AUC (0.912) was about the same compared to using the −3 to 3 range. The short cfDNA performed better than the long cfDNA.

FIG. 32B shows ROC curves for long, short, concatenated, and all molecules using positions 4, −2, −1, 1 and 4 aggregated across all CpG sites. Interestingly, the resulting AUC values were all less than the AUC values for using an ending ratio involving the same positions, namely a ratio of aggregating positions −4, −2, 1, and 4 and position −1, as shown in FIG. 29.

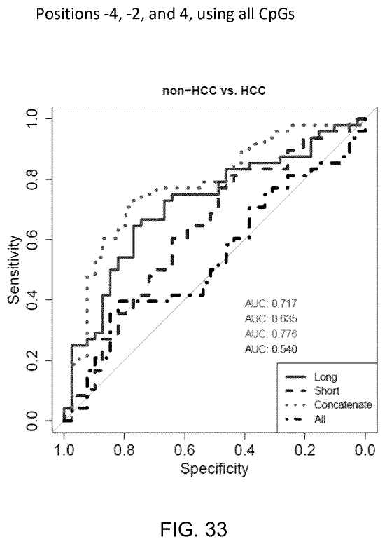

FIG. 33 shows ROC curves for long, short, concatenated, and all molecules using positions −4, −2, and 4 aggregated across all CpG sites. The SVM model for these positions performed the worse out of the sets of positions analyzed, but the concatenated AUC was still 0.776.

### 2. Ending Ratios

We also investigated ending ratios using various positions. We generated plots for all autosome CpGs. ‘Long+Short’ refers to calculating long and short molecule ending ratios separately, then adding ending ratios of long and short together. ‘All’ refers to long and short molecules were pooled together for ending ratio calculation. Combinations of additional positions include: (a) a first sum over position −4 and −2, and a second value for position −1, (b) a first sum over position −3 and 0, and a second value for position −1, (c) a first sum over position −4, −2, 1 and 4, and a second value for position 0, and (d) a first sum over position −4, −2, 1 and 4, and a second value for position −1 and 0.

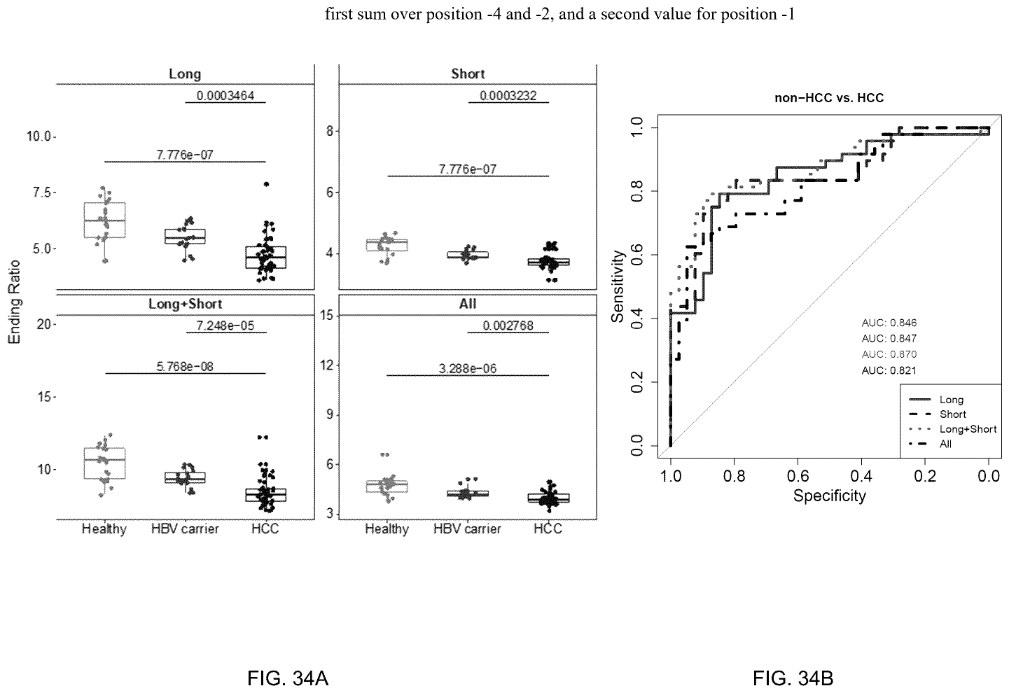

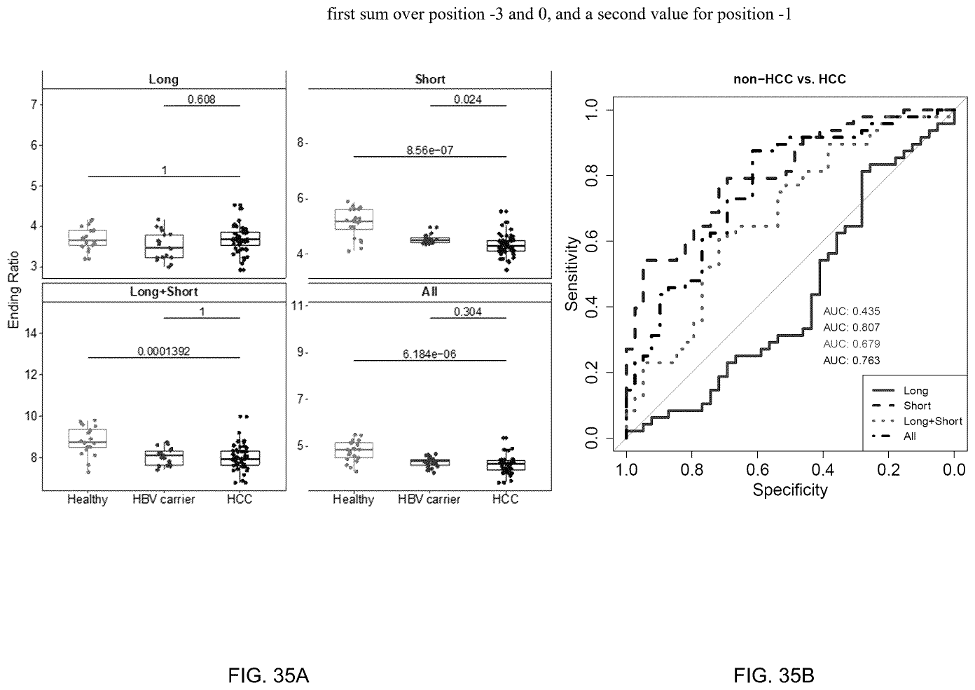

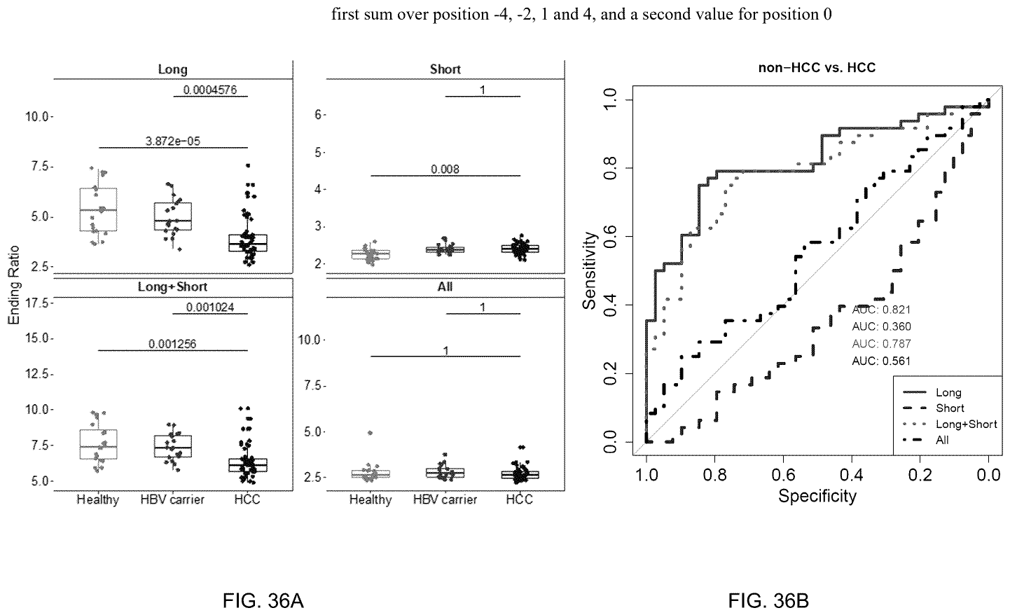

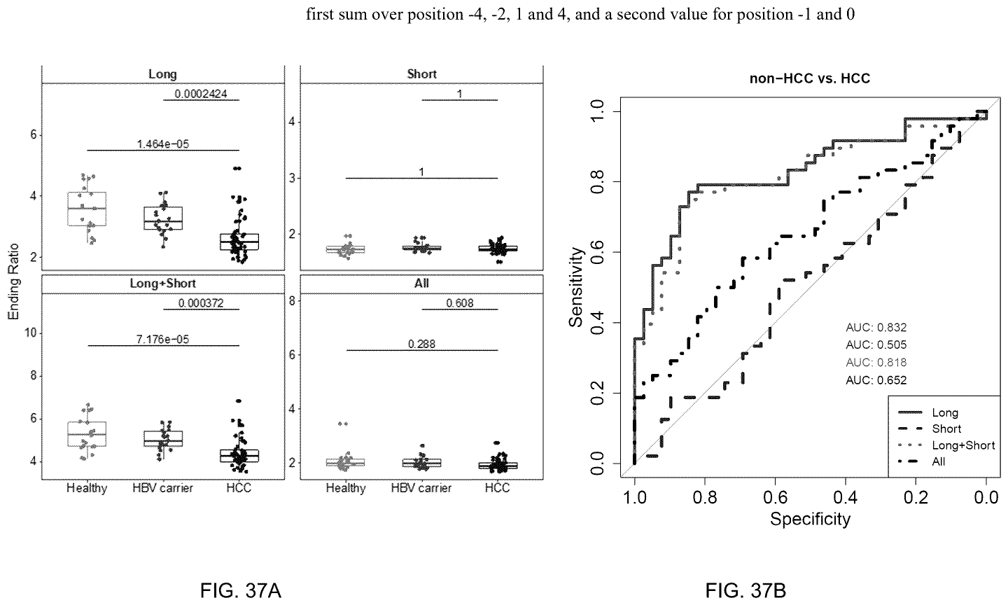

FIGS. 34A and 34B show results for differentiating HCC vs. non-HCC for all 26,752,699 autosome CpGs, using long, short, long+short, and all cfDNA fragments and a first sum over position −4 and −2, and a second value for position −1. FIGS. 35A and 35B show results for differentiating HCC vs. non-HCC for all 26,752,699 autosome CpGs, using long, short, long+short, and all cfDNA fragments and a first sum over position −3 and 0, and a second value for position −1. FIGS. 36A and 36B show results for differentiating HCC vs. non-HCC for all 26,752,699 autosome CpGs, using long, short, long+short, and all cfDNA fragments and a first sum over position −4, −2, 1 and 4, and a second value for position 0. FIGS. 37A and 37B show results for differentiating HCC vs. non-HCC for all 26,752,699 autosome CpGs, using long, short, long+short, and all cfDNA fragments and a first sum over position −4, −2, 1 and 4, and a second value for position −1 and 0.

The use of a first sum over position −4 and −2 and a second value for position −1, as shown in FIGS. 34A and 34B, achieved similar result as using a first sum over position −4, −2, 1 and 4 and a second value for position −1, as shown in FIG. 29. The other sets of positions showed decreased accuracy.

### E. Different Numbers of CpG Sites

We also investigated ending ratios using various numbers of CpG sites and various positions. We generated plots for randomly selected 15 million CpGs, 10 million CpGs, 5 million CpGs, one million CpGs and 500,000 CpGs. Combinations of positions include: (a) a first sum over position −4, −2, 1 and 4, and a second value for position −1, (b) a first sum over position −4 and −2, and a second value for position −1, (c) a first sum over position −3 and 0, and a second value for position −1, (d) a first sum over position −4, −2, 1 and 4, and a second value for position 0, and (e) a first sum over position −4, −2, 1 and 4, and a second value for position −1 and 0.

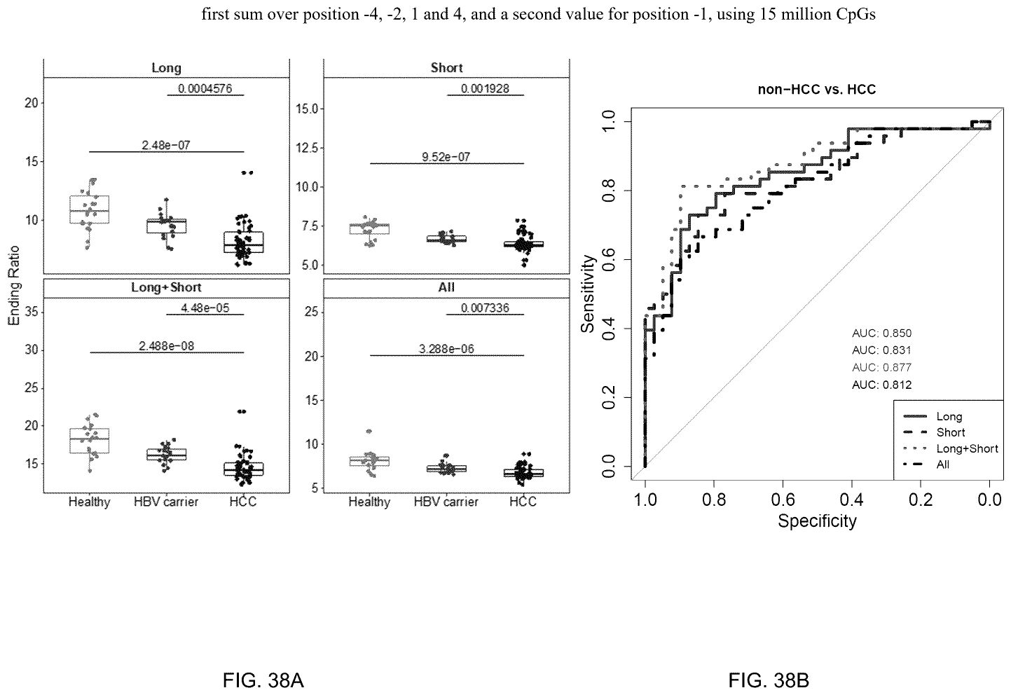

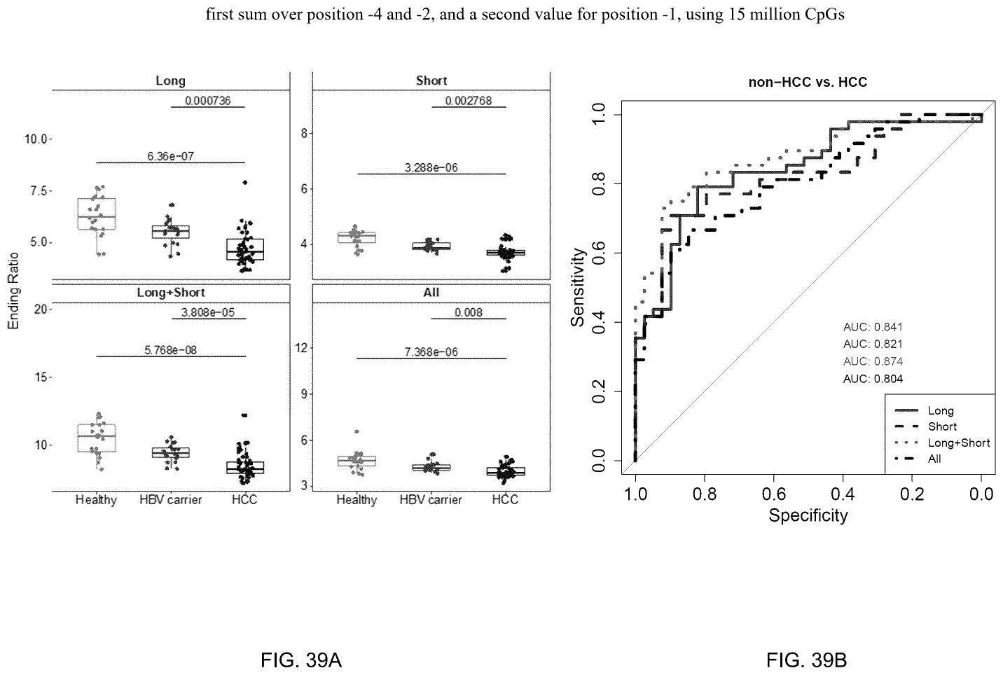

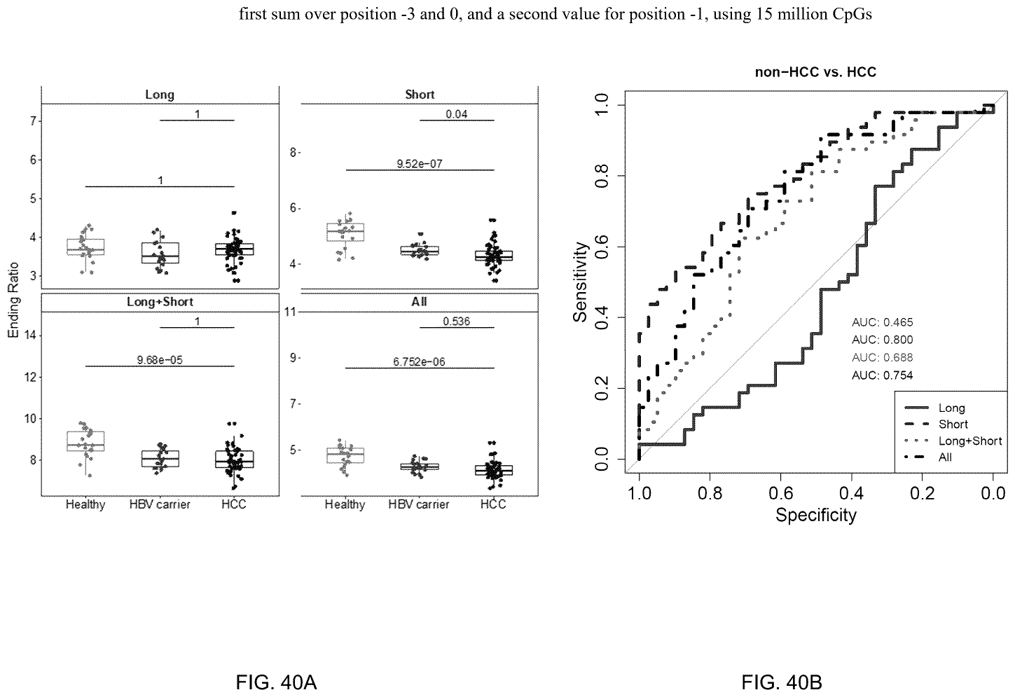

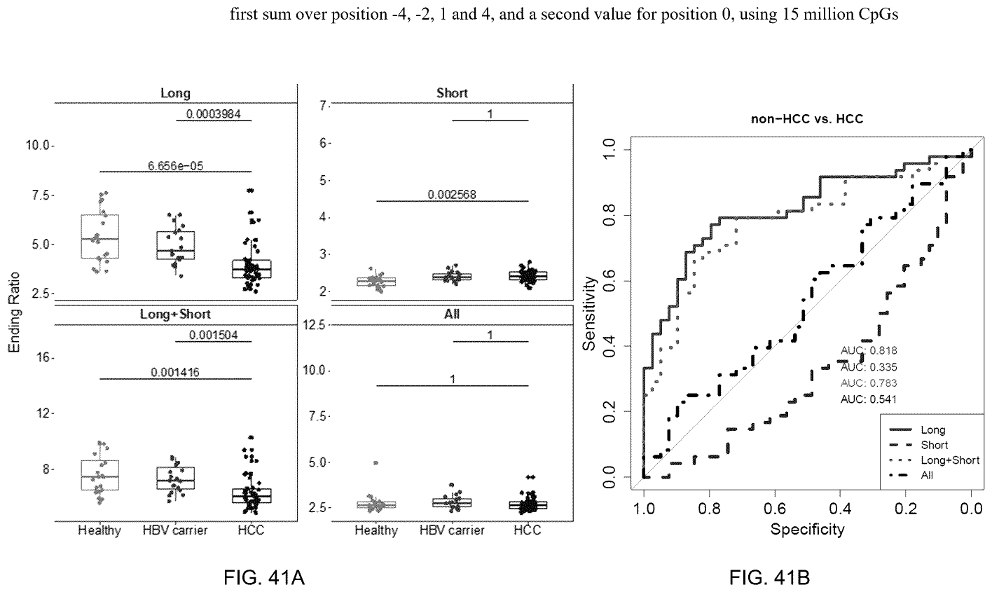

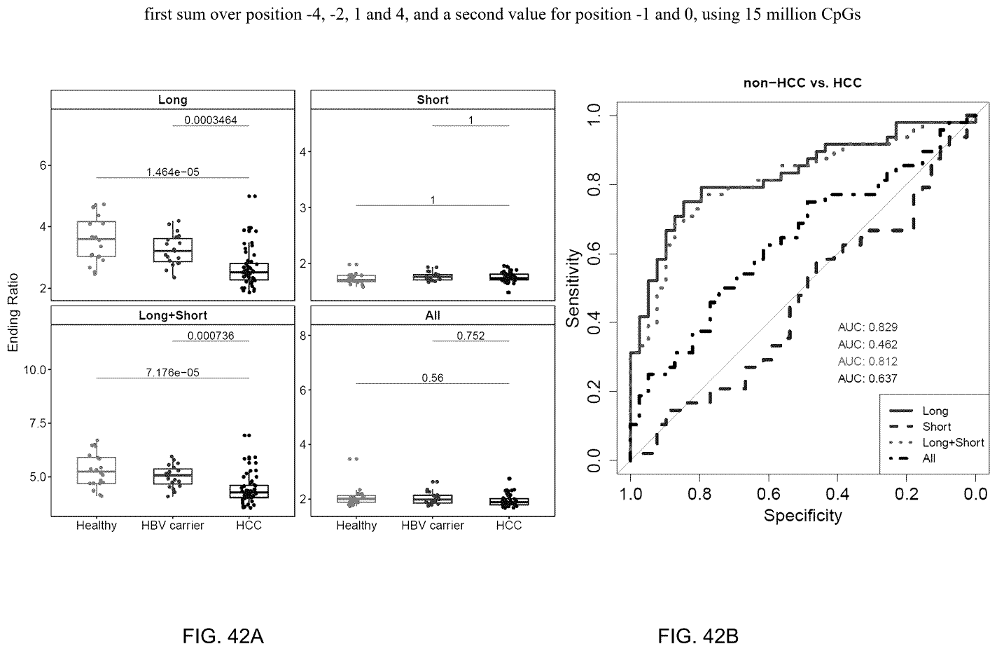

FIGS. 38A and 38B show results for differentiating HCC vs. non-HCC for randomly selected 15 million CpGs, using long, short, long+short, and all cfDNA fragments and a first sum over position −4, −2, 1 and 4, and a second value for position −1. FIGS. 39A and 39B show results for differentiating HCC vs. non-HCC for randomly selected 15 million CpGs, using long, short, long+short, and all cfDNA fragments and a first sum over position −4 and −2, and a second value for position −1. FIGS. 40A and 40B show results for differentiating HCC vs. non-HCC for randomly selected 15 million CpGs, using long, short, long+short, and all cfDNA fragments and a first sum over position −3 and 0, and a second value for position −1. FIGS. 41A and 41B show results for differentiating HCC vs. non-HCC for randomly selected 15 million CpGs, using long, short, long+short, and all cfDNA fragments and a first sum over position −4, −2, 1 and 4, and a second value for position 0. FIGS. 42A and 42B show results for differentiating HCC vs. non-HCC for randomly selected 15 million CpGs, using long, short, long+short, and all cfDNA fragments and a first sum over position −4, −2, 1 and 4, and a second value for position −1 and 0.

Using 15 million randomly selected CpG sites provided similar results as using all 26,752,699 autosome CpGs. The use of a first sum over position −4 and −2 and a second value for position −1 achieved similar result as using a first sum over position −4, −2, 1 and 4 and a second value for position −1. The other sets of positions showed decreased accuracy.

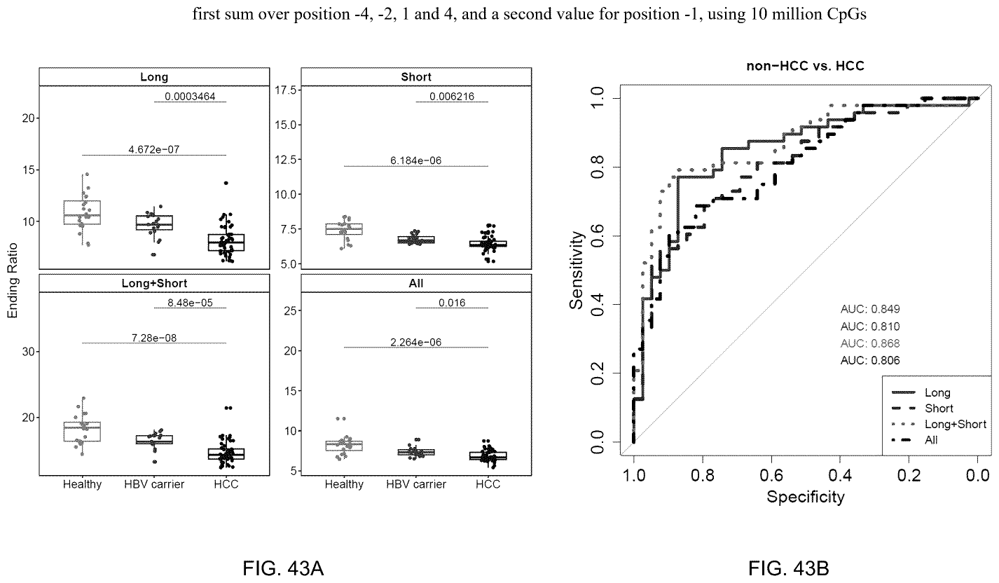

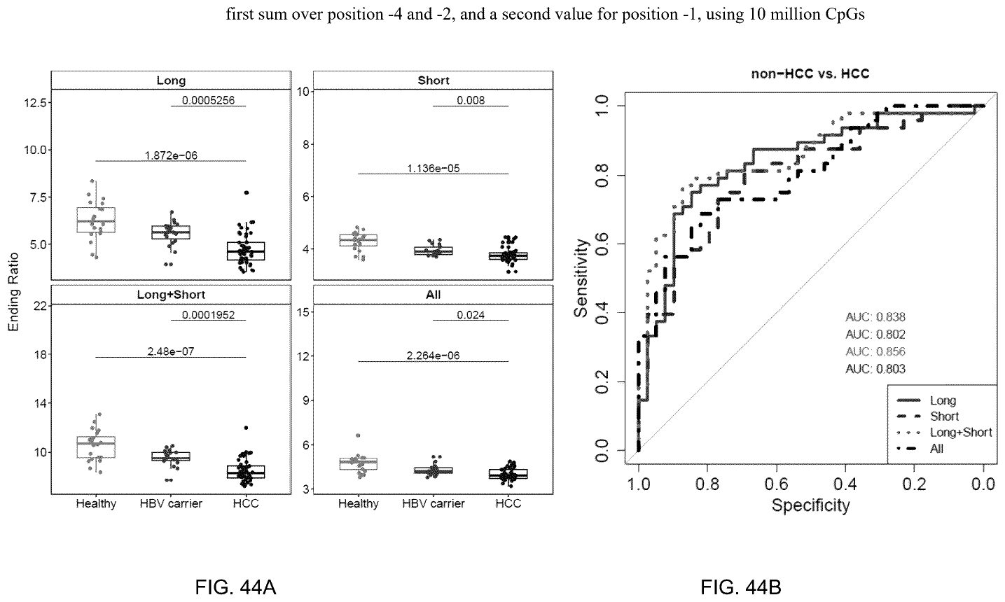

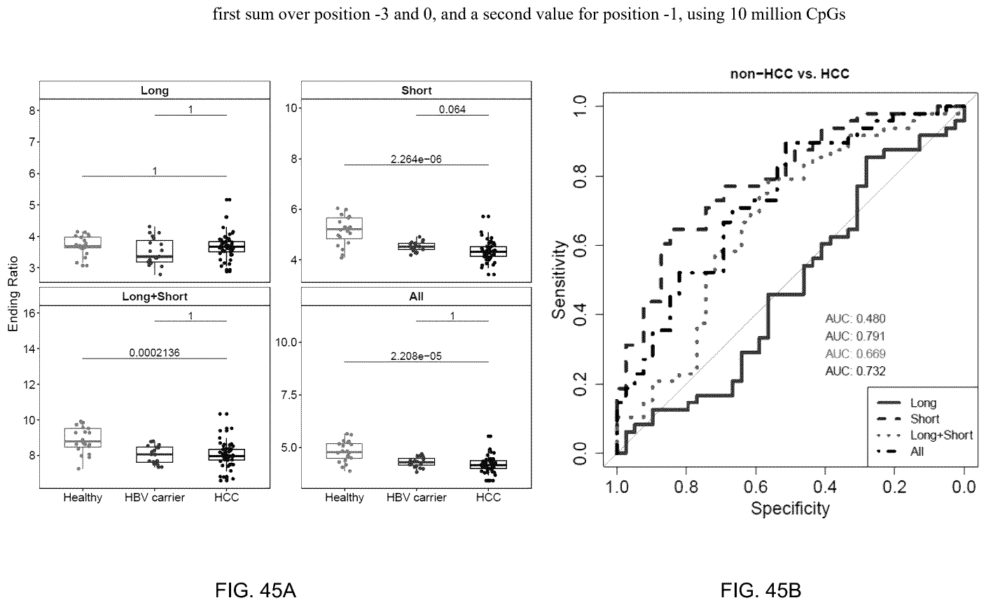

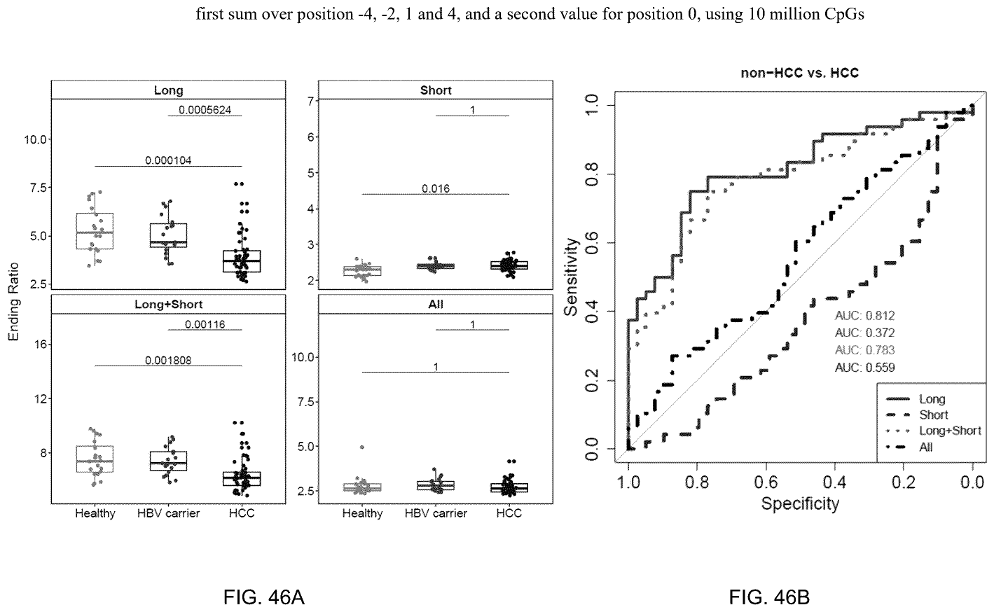

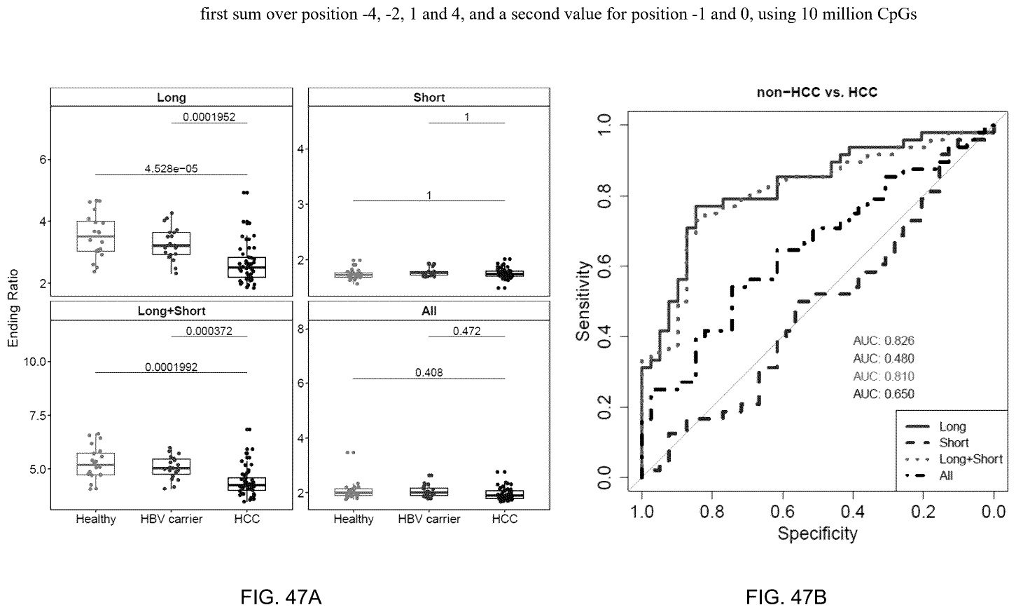

FIGS. 43A and 43B show results for differentiating HCC vs. non-HCC for randomly selected 10 million CpGs, using long, short, long+short, and all cfDNA fragments and a first sum over position −4, −2, 1 and 4, and a second value for position −1. FIGS. 44A and 44B show results for differentiating HCC vs. non-HCC for randomly selected 10 million CpGs, using long, short, long+short, and all cfDNA fragments and a first sum over position −4 and −2, and a second value for position −1. FIGS. 45A and 45B show results for differentiating HCC vs. non-HCC for randomly selected 10 million CpGs, using long, short, long+short, and all cfDNA fragments and a first sum over position −3 and 0, and a second value for position −1. FIGS. 46A and 46B show results for differentiating HCC vs. non-HCC for randomly selected 10 million CpGs, using long, short, long+short, and all cfDNA fragments and a first sum over position −4, −2, 1 and 4, and a second value for position 0. FIGS. 47A and 47B show results for differentiating HCC vs. non-HCC for randomly selected 10 million CpGs, using long, short, long+short, and all cfDNA fragments and a first sum over position −4, −2, 1 and 4, and a second value for position −1 and 0.

Using 10 million randomly selected CpG sites provided similar results as using all 26,752,699 autosome CpGs. The use of a first sum over position −4 and −2 and a second value for position −1 achieved similar result as using a first sum over position −4, −2, 1 and 4 and a second value for position −1. The other sets of positions showed decreased accuracy.

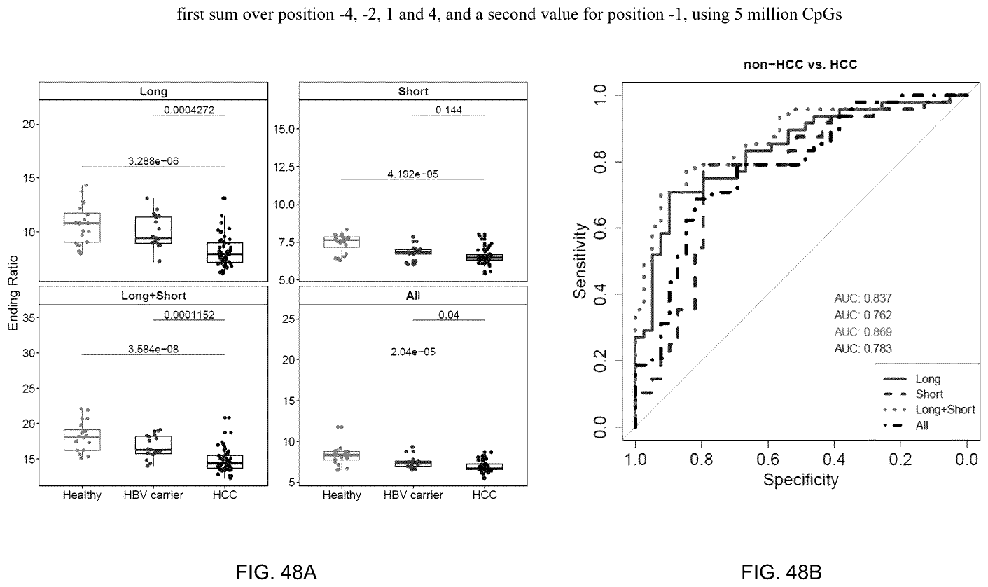

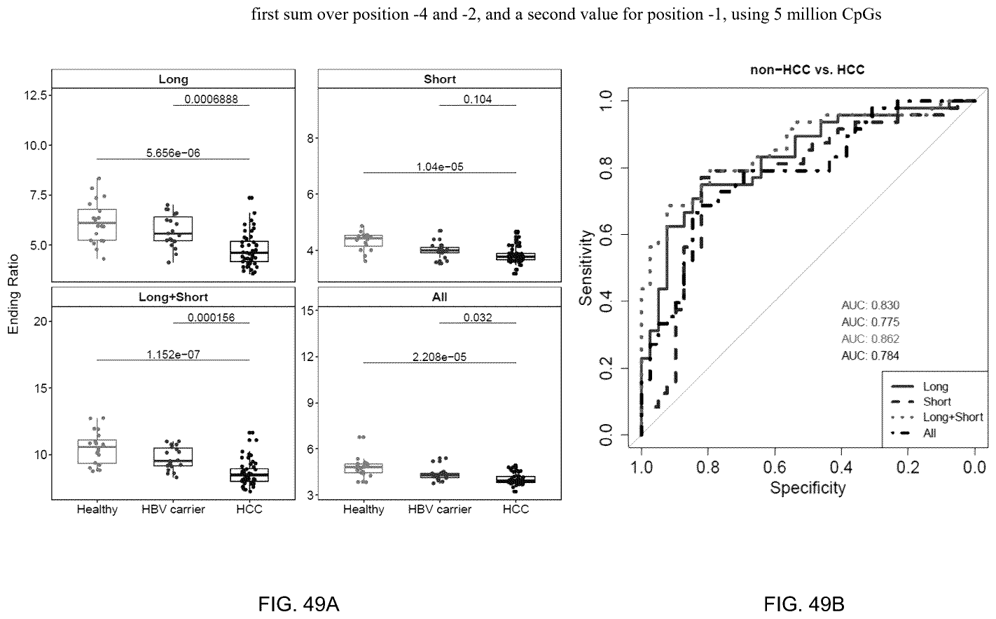

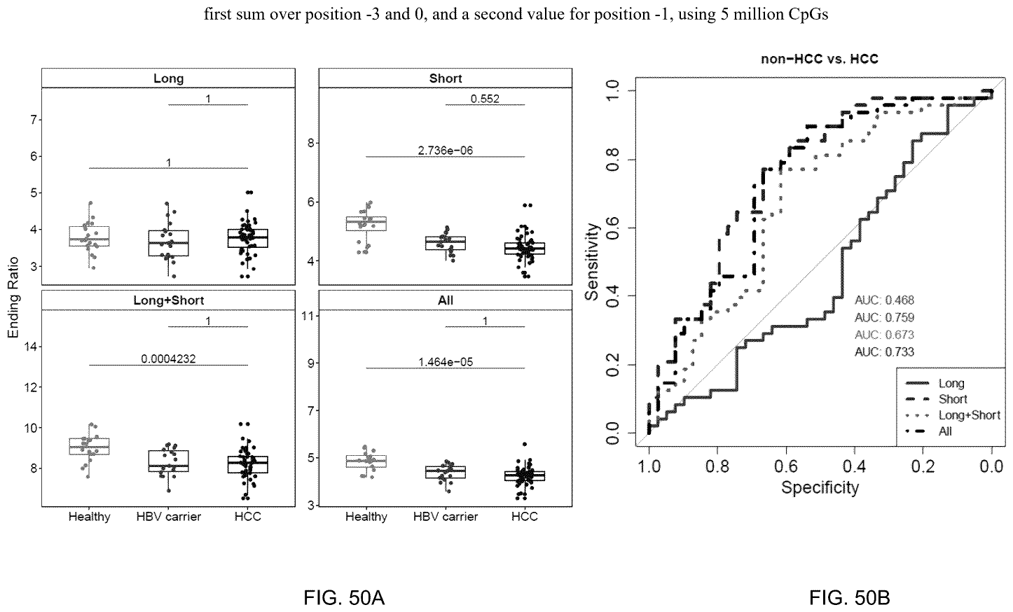

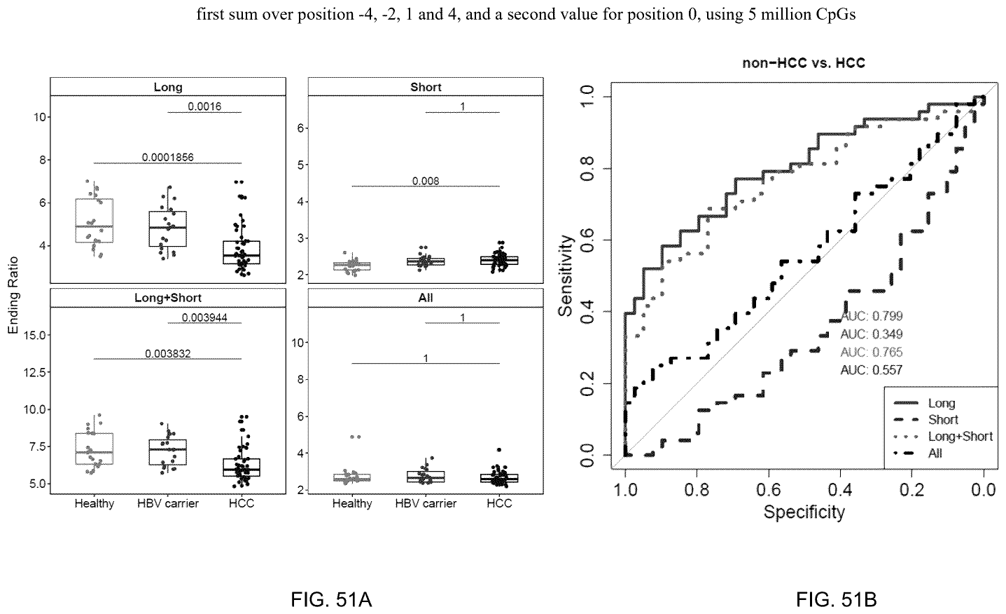

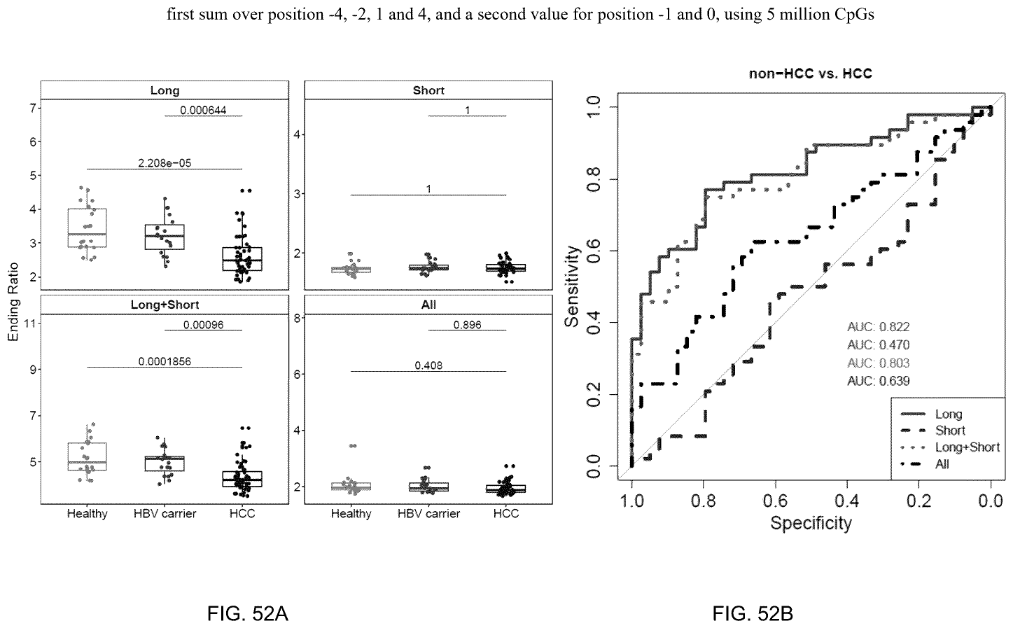

FIGS. 48A and 48B show results for differentiating HCC vs. non-HCC for randomly selected 5 million CpGs, using long, short, long+short, and all cfDNA fragments and a first sum over position −4, −2, 1 and 4, and a second value for position −1. FIGS. 49A and 49B show results for differentiating HCC vs. non-HCC for randomly selected 5 million CpGs, using long, short, long+short, and all cfDNA fragments and a first sum over position −4 and −2, and a second value for position −1. FIGS. 50A and 50B show results for differentiating HCC vs. non-HCC for randomly selected 5 million CpGs, using long, short, long+short, and all cfDNA fragments and a first sum over position −3 and 0, and a second value for position −1. FIGS. 51A and 51B show results for differentiating HCC vs. non-HCC for randomly selected 5 million CpGs, using long, short, long+short, and all cfDNA fragments and a first sum over position −4, −2, 1 and 4, and a second value for position 0. FIGS. 52A and 52B show results for differentiating HCC vs. non-HCC for randomly selected 5 million CpGs, using long, short, long+short, and all cfDNA fragments and a first sum over position −4, −2, 1 and 4, and a second value for position −1 and 0.

Using 5 million randomly selected CpG sites provided similar results as using all 26,752,699 autosome CpGs for the long and long+short cfDNA. The short cfDNA accuracy starts to decrease at this point, as well as using all of the cfDNA. The use of a first sum over position −4 and −2 and a second value for position −1 achieved similar result as using a first sum over position −4, −2, 1 and 4 and a second value for position −1. The other sets of positions showed decreased accuracy, although the long cfDNA and long+short showed improved results for first sum over position −4, −2, 1 and 4, and a second value for position 0 and first sum over position −4, −2, 1 and 4, and a second value for position −1 and 0.

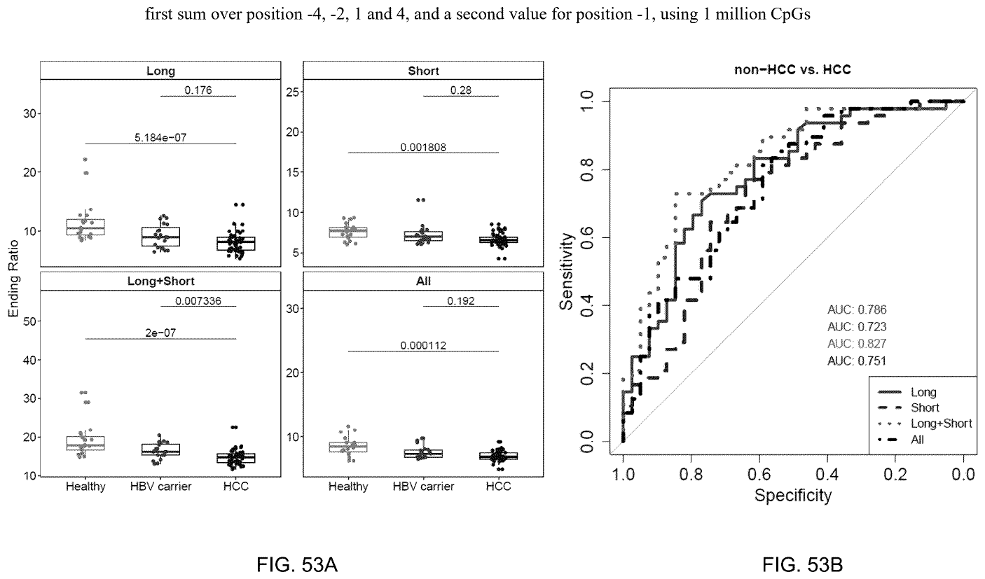

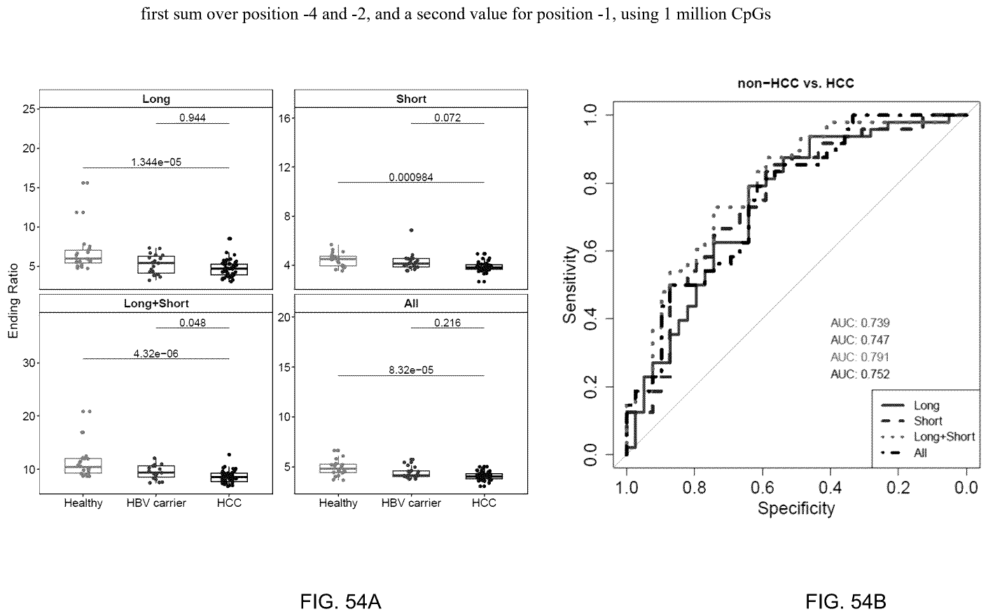

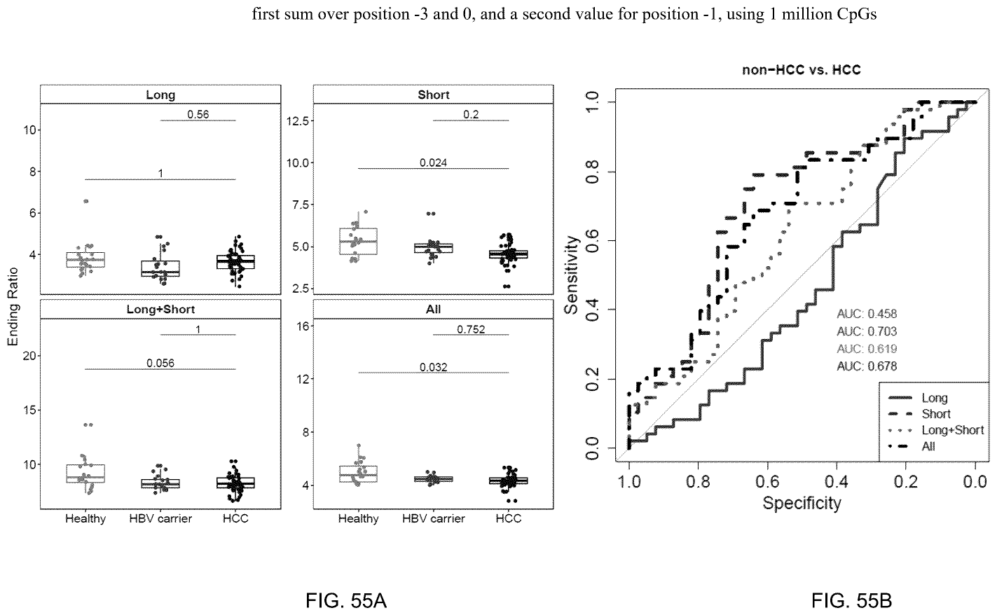

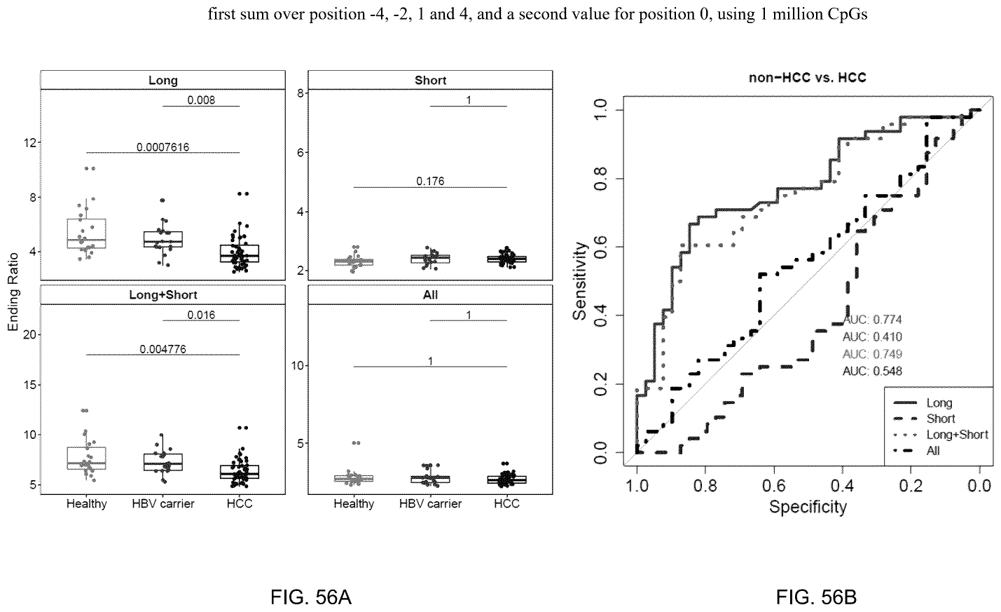

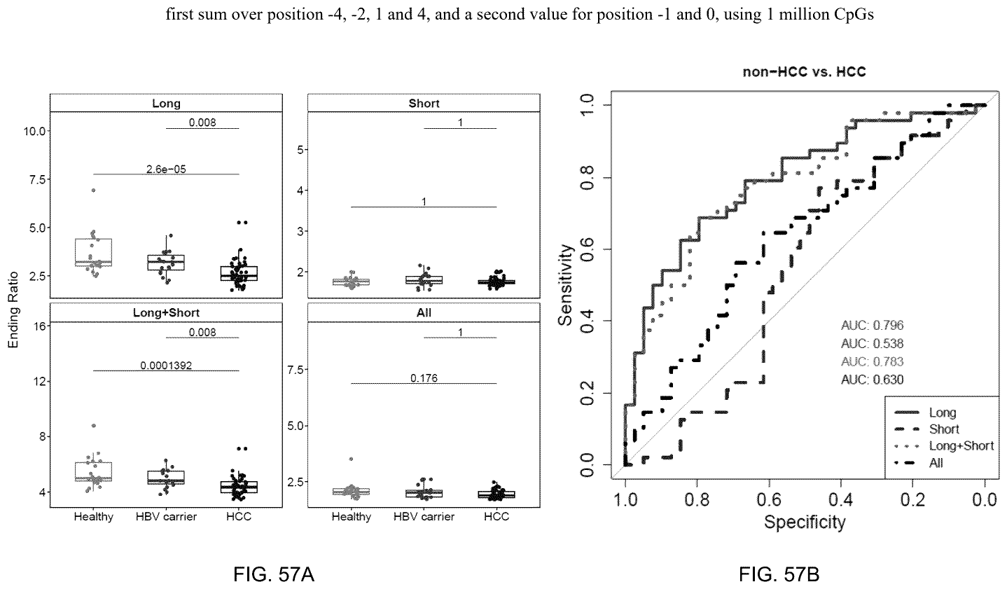

FIGS. 53A and 53B show results for differentiating HCC vs. non-HCC for randomly selected 1 million CpGs, using long, short, long+short, and all cfDNA fragments and a first sum over position −4, −2, 1 and 4, and a second value for position −1. FIGS. 54A and 54B show results for differentiating HCC vs. non-HCC for randomly selected 1 million CpGs, using long, short, long+short, and all cfDNA fragments and a first sum over position −4 and −2, and a second value for position −1. FIGS. 55A and 55B show results for differentiating HCC vs. non-HCC for randomly selected 1 million CpGs, using long, short, long+short, and all cfDNA fragments and a first sum over position −3 and 0, and a second value for position −1. FIGS. 56A and 56B show results for differentiating HCC vs. non-HCC for randomly selected 1 million CpGs, using long, short, long+short, and all cfDNA fragments and a first sum over position −4, −2, 1 and 4, and a second value for position 0. FIGS. 57A and 57B show results for differentiating HCC vs. non-HCC for randomly selected 1 million CpGs, using long, short, long+short, and all cfDNA fragments and a first sum over position −4, −2, 1 and 4, and a second value for position −1 and 0.

Using 1 million randomly selected CpG sites provided similar results as using all 26,752,699 autosome CpGs for the long+short cfDNA. The long cfDNA accuracy also starts to decrease at this point. The use of a first sum over position −4 and −2 and a second value for position −1 achieved better results than using a first sum over position −4, −2, 1 and 4 and a second value for position −1.

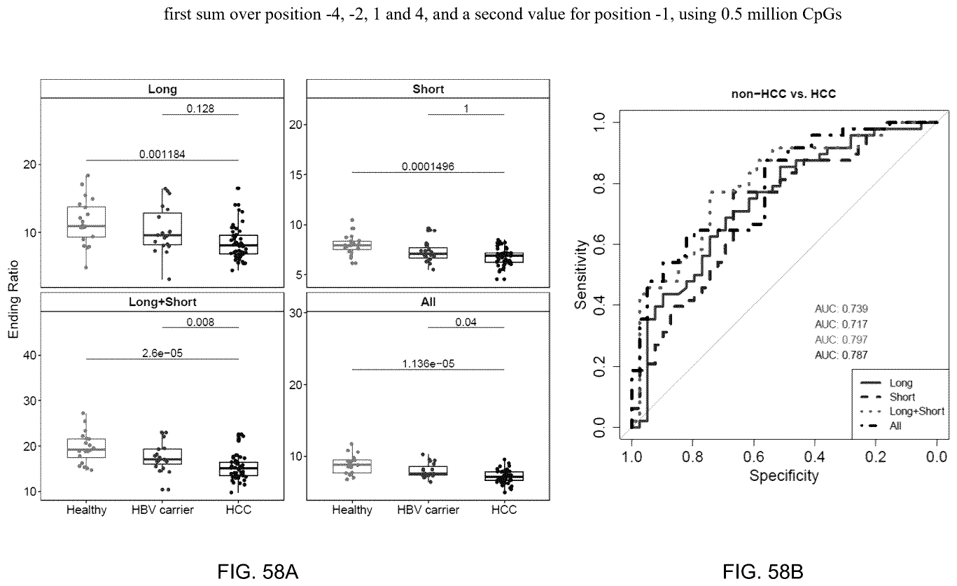

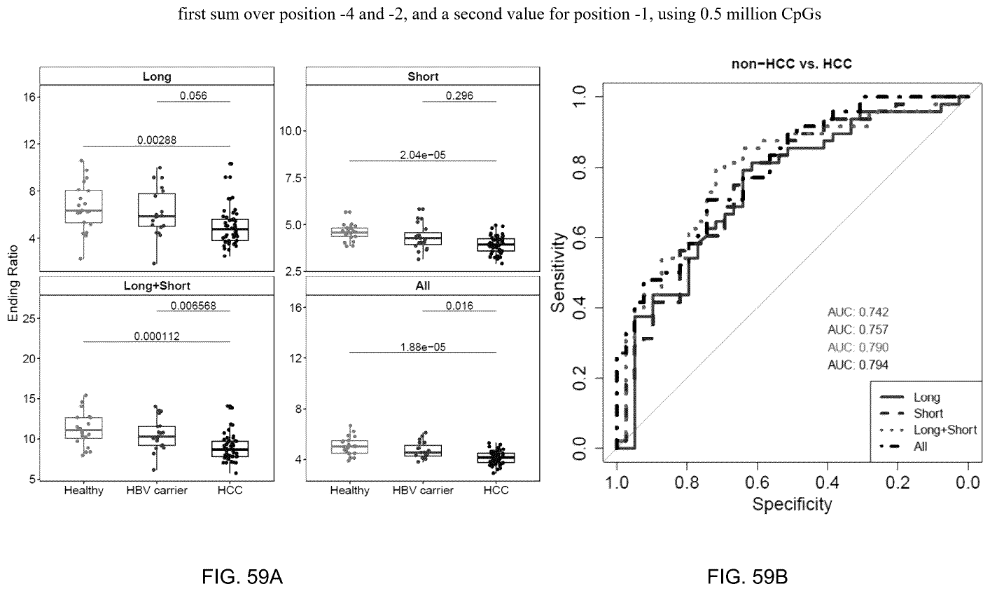

FIGS. 58A and 58B show results for differentiating HCC vs. non-HCC for randomly selected 0.5 million CpGs, using long, short, long+short, and all cfDNA fragments and a first sum over position −4, −2, 1 and 4, and a second value for position −1. FIGS. 59A and 59B show results for differentiating HCC vs. non-HCC for randomly selected 0.5 million CpGs, using long, short, long+short, and all cfDNA fragments and a first sum over position −4 and −2, and a second value for position −1. FIGS. 60A and 60B show results for differentiating HCC vs. non-HCC for randomly selected 0.5 million CpGs, using long, short, long+short, and all cfDNA fragments and a first sum over position −3 and 0, and a second value for position −1. FIGS. 61A and 61B show results for differentiating HCC vs. non-HCC for randomly selected 0.5 million CpGs, using long, short, long+short, and all cfDNA fragments and a first sum over position −4, −2, 1 and 4, and a second value for position 0. FIGS. 62A and 62B show results for differentiating HCC vs. non-HCC for randomly selected 0.5 million CpGs, using long, short, long+short, and all cfDNA fragments and a first sum over position −4, −2, 1 and 4, and a second value for position −1 and 0.

The accuracy at 500,000 cfDNA fragments showed similar behavior with the best results arising from the use of a first sum over position −4 and −2 and a second value for position −1 and using a first sum over position −4, −2, 1 and 4 and a second value for position −1.

### F. CRC

To illustrate that this technique works for different cancers, not just HCC, we also investigated colorectal cancer (CRC).

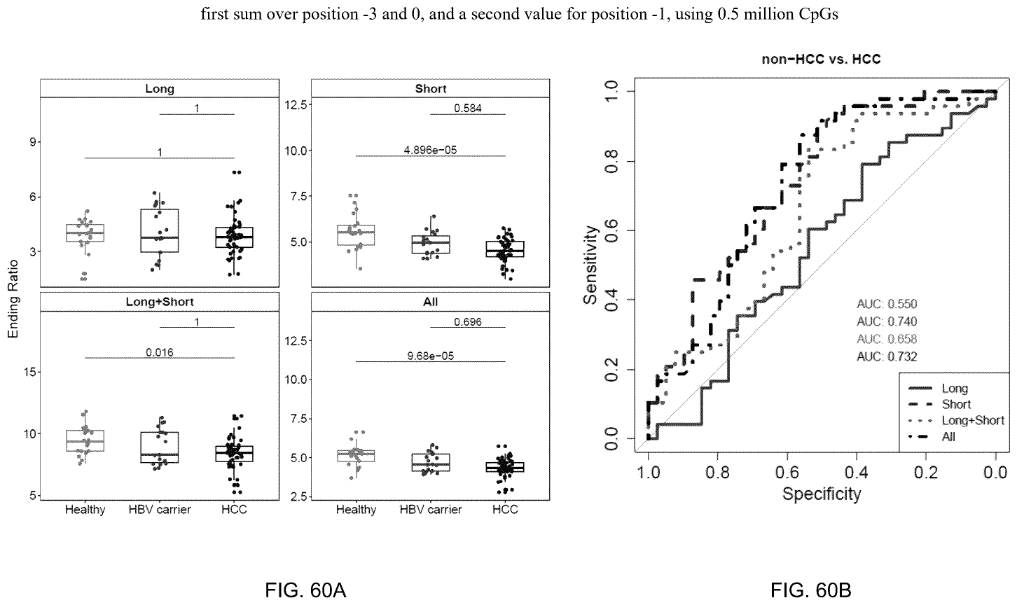

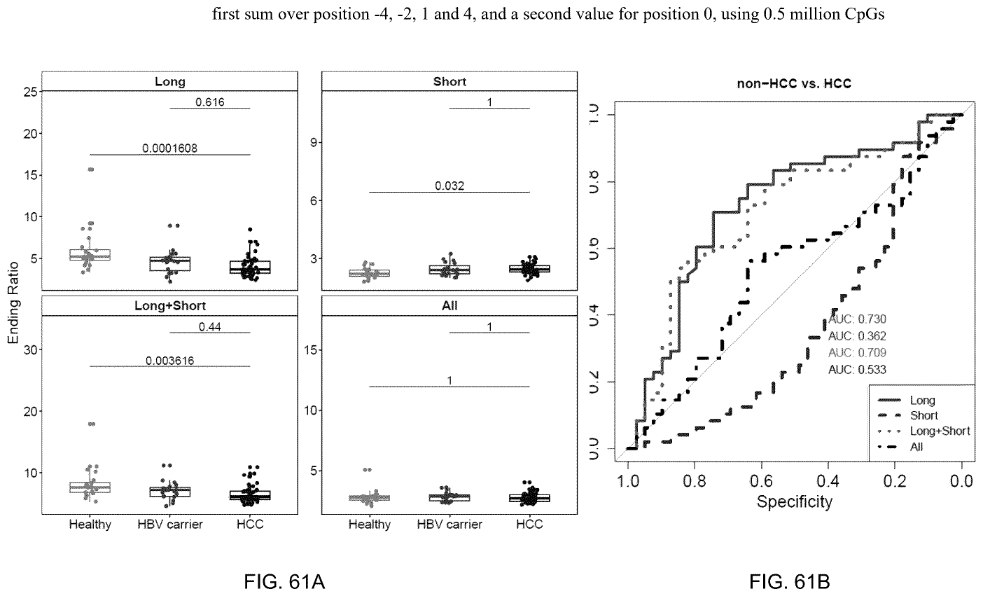

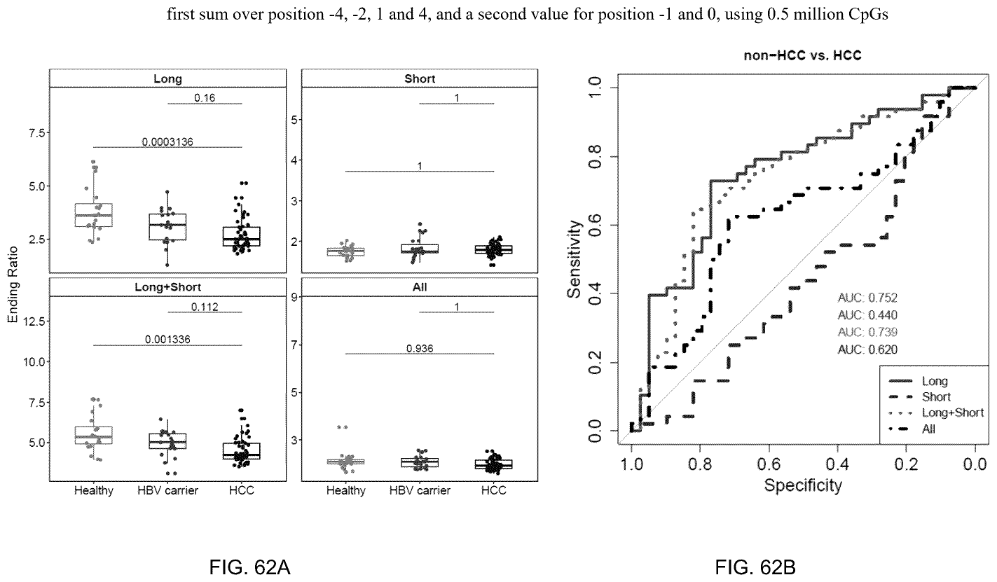

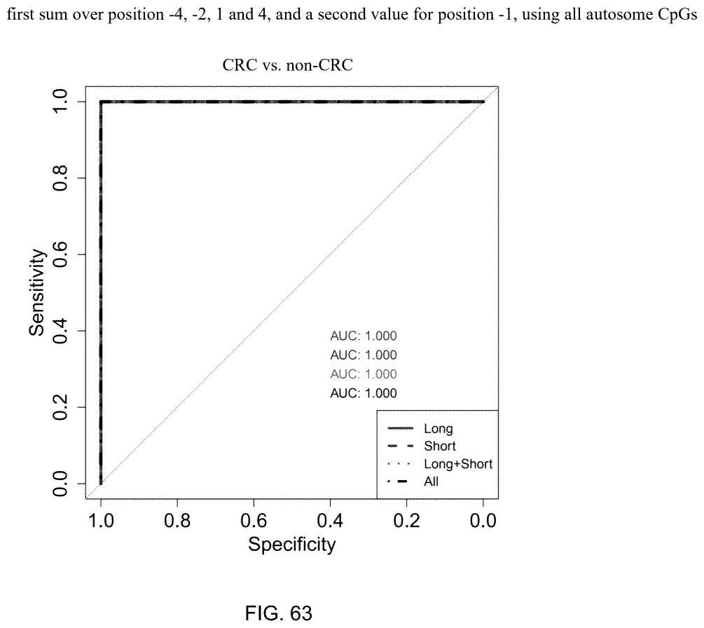

FIG. 63 is an ROC curve for differentiating CRC vs. non-CRC for 26,752,699 autosome CpGs, using long, short, long+short, and all cfDNA fragments and a first sum over position −4, −2, 1 and 4, and a second value for position −1. For CRC with the 4 samples, we can distinguish them from healthy samples perfectly using ending ratio of −4, −2, 1 and 4 to −1 from all autosome CpGs.

### G. Method

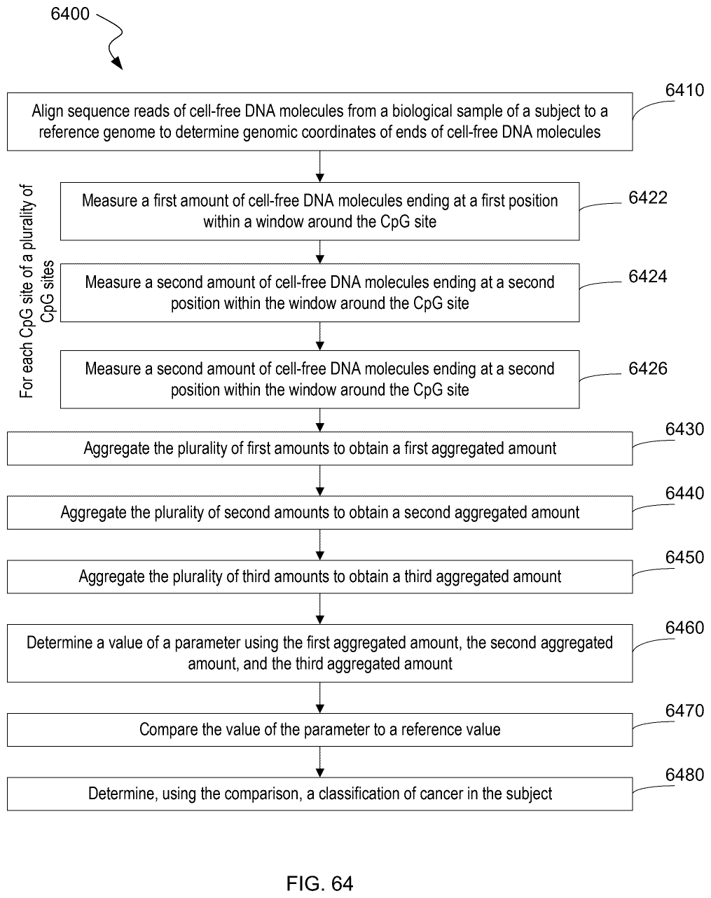

FIG. 64 is a flowchart of an example process 6400 for detecting cancer using cleavage profiles aggregated at various types of CpG sites. In some implementations, one or more process blocks of FIG. 64 may be performed by measurement systems, computer systems, or any system described herein. As described above, an ending ratio or a model can be used to determine whether a subject has cancer. The ending ratio is a ratio of amounts of fragments having an end at various positions around a CpG site to amounts of fragments having an end at other positions around the CpG site. Long and/or short cfDNA may also be used for the ending ratios. Various number of CpG sites may be used, e.g., at least 50, 100, 200, 500, 1,000, 5,000, 10,000, 50,000, 100,000, 500,000, one million, 5 million, 10 million, or 15 million.

At block 6410, sequence reads of cell-free DNA molecules from a biological sample of a subject are aligned to a reference genome to determine genomic coordinates of ends of the cell-free DNA molecules. The cell-free DNA molecules can be long cell-free DNA molecules and have sizes equal to or above a first cutoff value of at least 400 nt. Alternatively or in addition, the cell-free DNA molecules or an additional set of cell-free DNA molecules can be short cell-free DNA molecules, which can have sizes equal to or below a second cutoff value. The second cutoff value can be 500 nt or less. If both sets of cfDNA molecules are used, the second cutoff value can be less than the first cutoff value although it can be greater.

At block 6422, a first amount of cell-free DNA molecules ending at a first position within a window around the CpG site is measured for each CpG site of a plurality of CpG sites. The measurement uses the genomic coordinates previously determined. The cell-free DNA molecules can be identified using 3′, 5′, or both ends of the cell-free DNA molecules.

The plurality of CpG sites may include one or more hypermethylated CpG sites and one or more hypomethylated CpG sites. The window may include positions −5 to +5 relative to the CpG site. In some embodiments, the window may include positions −10 to +10 relative to the CpG site. The window may include positions from −n to +m, where n and m include the integers from 1 to 100 (e.g., including 20, 30, 40, 50). The window may be symmetrical or asymmetrical relative to the CpG site.

At block 6424, a second amount of cell-free DNA molecules ending at a second position within the window around the CpG site is measured for each CpG site of the plurality of CpG sites.

At block 6426, a third amount of cell-free DNA molecules ending at a third position within the window around the CpG site is measured for each CpG site of the plurality of CpG sites.

In some embodiments, the first position, the second position, and the third position may each be at one of the positions −4, −2, −1, 1, or 4.

The first amount of cell-free DNA molecules may include of cell-free DNA molecules having a 5′ end at the first position. The second amount of cell-free DNA molecules may include of cell-free DNA molecules having a 5′ end at the second position. The third amount of cell-free DNA molecules may include of cell-free DNA molecules having a 5′ end at the third position. In some embodiments, the first amount, the second amount, and the third amount may include the 3′ end at the respective positions in place of or in addition to the 5′ end.

At block 6430, the plurality of first amounts is aggregated to obtain a first aggregated amount. An aggregated amount may be a sum or a weighted sum.

At block 6440, the plurality of second amounts is aggregated to obtain a second aggregated amount.

At block 6450, the plurality of third amounts is aggregated to obtain a third aggregated amount.

At block 6460, a value of a parameter is determined using the first aggregated amount, the second aggregated amount, and the third aggregated amount. For example, the aggregated amounts may be summed together, multiplied together, or added in a linear combination (e.g., weighted sum). Additional positions and additional aggregated amounts may be used for the sum or product or ratio. For example, aggregated amounts from positions at −4, −2, +1, and +4 may be used.

As other examples, some of the aggregated amounts can be summed and ratio taken of one or more other aggregated amounts. For example, an aggregated amount for position −1 or a sum aggregated amounts for positions −1 and 0 can be used to determine a normalized sum. Accordingly, the parameter can include a normalized sum that is determined by determining a sum that includes the first aggregated amount and the second aggregated amount and normalizing the sum using the third aggregated amount. Additional aggregated amounts may be used when determining the value of the parameter.

The value of the parameter may be determined using additional aggregated amounts. For example, determining the value of the parameter may include measuring a fourth amount of cell-free DNA molecules ending at a fourth position within the window around the CpG site for each CpG site of the plurality of CpG sites, thereby determining a plurality of fourth amounts. The plurality of fourth amounts may be aggregated to obtain a fourth aggregated amount. The first aggregated amount, the second aggregated amount, and the third aggregated amount may be normalized using the fourth aggregated amount. The fourth position may be −1 or any integer in the window (e.g., −5, −4, −3 −2, −1, 0, +1, +2, +3, +4, +5). The value of the parameter can be a normalized sum of the first aggregated amount, the second aggregated amount, the third aggregated amount, and the fourth aggregated amount, e.g., where the first position is −4, the second position is −2, the third position is +1, and the fourth position is +4.

The parameter may be a normalized parameter, e.g., by including a normalized sum. As another example, each of the first aggregated amount, the second aggregated amount, and the third aggregated amount are normalized before determining the value of the parameter. This normalization can be performed in various ways, e.g., by normalizing using all sequence reads or by normalizing each amount for each window around a respective CpG site before aggregating to determine the aggregated amounts.

At block 6470, the value of the parameter is compared to a reference value. The reference value may be determined using reference samples (also referred to as training samples) from subjects with a known level of a cancer (e.g., known classifications of cancer) or from healthy subjects. The reference values may be selected as a value that provides a tradeoff between sensitivity and specificity for discriminating between different classifications of cancer.

When both long and short cfDNA are used, two different normalized sums can be used, where the two separate parameters can be compared to two separate reference values or combined (e.g., summed) to determine the value of the parameter. For instance, a first normalized sum can be determined for cell-free DNA molecules having sizes above a first cutoff value, wherein the first cutoff value is of at least 400 nt. The parameter can further include a second normalized sum (1) determined for the first position, the second position, and the third position and (2) determined for cell-free DNA molecules having sizes equal to or less than a second cutoff value. The second cutoff value can be equal to or less than the first cutoff value.

At block 6480, a classification of cancer in the subject is determined using the comparison. The cancer may be hepatocellular carcinoma, colorectal cancer, carcinoma, sarcoma, lymphoma, leukemia, or any cancer of any tissue or organ. Organs may be in the musculoskeletal system, digestive system, respiratory system, urinary system, reproductive systems, endocrine system, circulatory system, nervous system, or integumentary system. Organs may include the skeleton, stomach, small intestine, large intestine, rectum, liver, gallbladder, mesentery, pancreas, lungs, kidneys, ureter, bladder, urethra, ovaries, testicles, prostate, thyroid, lymph node, spleen, brain, breast, or skin. Tissues may include bone or blood. The classification may be the existence/absence of cancer or a severity of cancer. The cancer may be determined to exist if the value of the parameter exceeds the reference value.

In some embodiments, the value of the parameter may be determined using a model, including any machine learning model (e.g., support vector machine model described with FIGS. 13A and 13B). Determining the value of the parameter may include inputting the first aggregated amount, the second aggregated amount, and the third aggregated amount into a model as part of a feature vector. The model can then perform the comparing of the value of the parameter to the reference value.

The model may be trained by receiving training input data. The training input data may include data for each training sample of a plurality of training samples. For each CpG site of the plurality of CpG sites, a first training amount of cell-free DNA molecules ending at the first position within the window around the CpG site, a second training amount of cell-free DNA molecules ending at the second position within the window around the CpG site, and a third training amount of cell-free DNA molecules ending at the third position within the window around the CpG site may be included. In some embodiments, the training input data may include aggregated amounts at the positions. Process 6400 may include storing a training set that may include the training input data and a label for each training sample. The label may indicate a known classification of cancer of a training subject associated with the training sample. Process 6400 may include optimizing, using the training set, model parameters based on outputs of the model matching or not matching corresponding labels when the training input data is input to the model. The model may output the value of the parameter. The model may perform the comparing of the value of the parameter to the reference value. The reference value may be a probability threshold for a certain classification. The parameter may be an estimated probability for the classification calculated by the model.

The model can use concatenated data for long and short cfDNA fragments, as described above. Accordingly, a first group can be of long cell-free DNA molecules can have sizes above a first cutoff value. The first cutoff value can be at least 400 nt. A second group can be of short cfDNA molecules. Process 6400 can include: measuring a fourth aggregated amount for a group of short cell-free DNA molecules at the first position; measuring a fifth aggregated amount for the group of short cell-free DNA molecules at the second position; and measuring a sixth aggregated amount for the group of short cell-free DNA molecules at the third position. The short cell-free DNA molecules can have sizes equal to or less than a second cutoff value. The second cutoff value can be equal to or less than the first cutoff value, but could be greater than the first cutoff value. Such a relationship among size cutoffs for long and short cfDNA fragments can apply to all of the techniques described herein.

The model can use various positions, with an aggregated amount for each position (potentially two: one for long cfDNA and one for short cfDNA) input into the model. For instance, 11 positions can be used (−5 to +5). Examples of other possible sets of positions are provided herein. As an example, an input feature vector for the model can comprise aggregated amounts of cell-free DNA molecules ending at at least five positions within the window. The window can consist of 11 positions or less centered around the CpG site.

Process 6400 may include additional implementations, such as any single implementation or any combination of implementations described herein and/or in connection with one or more other processes described elsewhere herein.

Although FIG. 64 shows example blocks of process 6400, in some implementations, process 6400 may include additional blocks, fewer blocks, different blocks, or differently arranged blocks than those depicted in FIG. 64. Additionally, or alternatively, two or more of the blocks of process 6400 may be performed in parallel.

### VIII. NUCLEASE ACTIVITY

We also investigated nuclease activity and relation to cfDNA fragment size.

### A. Investigation of Long Molecules Generation Using Knock-Out Mice

The amount of cfDNA molecules with 5′ ending in an A or G was reported to increase as the size of cfDNA molecules increased (Yu et al. 2021) and the nuclease DNA fragmentation factor subunit beta (DFFB) exhibited a preference to cut 5′ to an A or G nucleotide (Han et al. 2020). Therefore, to gain potential mechanistic insights on generating long cfDNA molecules in human plasma, we studied mouse models in which different nuclease genes had been knocked out. We sequenced 4 wild-type (WT), 5 Dffb^{-/-}, 5 Dnase1^{-/-}, 5 Dnase1l3^{-/-}, and 5 Dnase1^{-/-} and Dnase1l3^{-/-} double knockout mice samples, with each sample pooled from 3-5 mice subjects, using SMRT sequencing.

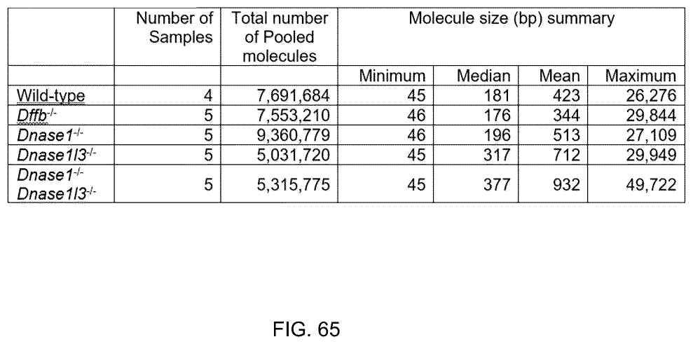

FIG. 65 shows a table of samples analyzed from the wild-type (WT), 5 Dffb^{-/-}, 5 Dnase1^{-/-}, 5 Dnase1l3^{-/-}, and 5 Dnase1^{-/-} and Dnase1l3^{-/-} double knockout mice.

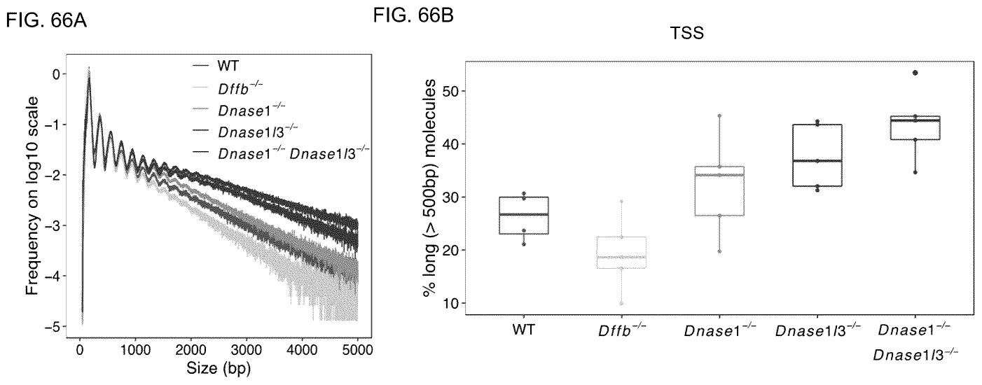

FIG. 66A shows pooled molecule size distributions of wild-type and nuclease-deficient mice sequenced using PacBio sequencing platform. Visualization of size in the range of 0-5000 bp and log 10-transformed frequencies were used.

We first analyzed the size profile of plasma cfDNA from these mice by pooling samples of the same genotype. Compared with wild-type mice, the frequency of long cfDNA molecules above 1 kb in Dffb^{-/-} mice consistently decreased, whereas the corresponding frequency in Dnase1^{-/-} mice increased, and such an increasing pattern was further enhanced in Dnase1l3^{-/-} mice and mice with double deletion of Dnase1 and Dnase1l3 (Dnase1^{-/-} & Dnase1l3^{-/-}). For instance, the percentages of cfDNA molecules with a size of >1 kb were 10.2%, 6.6%, 14.2%, 21.8% and 28.8% in the wild-type, Dffb^{-/-}, Dnase1^{-/-}, Dnase1l3^{-/-}, and Dnase1^{-/-}& Dnase1l3^{-/-} mice, respectively. The data suggested that DFFB was at least in part responsible for the generation of long cfDNA molecules. The removal of DNASE1L3 and DNASE1 would lead to an enhanced effect of generation of long cfDNA molecules, as DFFB might, in the absence of DNASE1L3 and DNASE1, act as a dominant nuclease in contributing cfDNA into circulation.

FIG. 66B shows a boxplot of proportions of long molecules (>500 bp) originated from 2000 bp upstream and downstream of transcription starting sites for different subjects that lack certain nucleases.

Interestingly, the deletion of Dffb resulted in the reduction of long molecules originating from regions around TSSs which normally represented the open chromatin states, further providing evidence that DFFB might be responsible for the patterns related to long cfDNA molecules. The increase of long cfDNA molecules in regions nearby TSSs was elevated in mice where DFFB functioned prominently by knocking out Dnase1 and Dnase1l3.

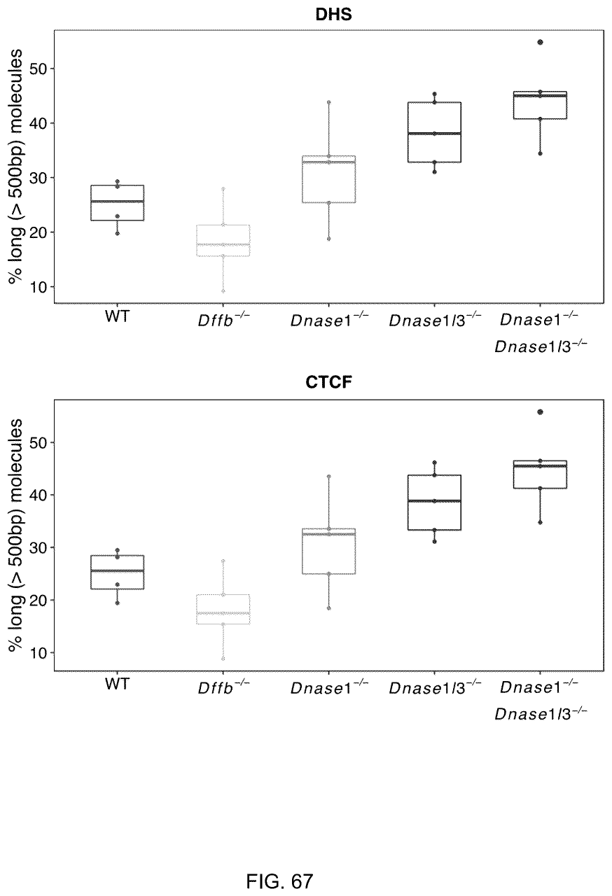

FIG. 67 illustrates boxplots showing proportions of long molecules originated from 2000 bp upstream and downstream of DHSs and CTCFs for different subjects that lack certain nucleases. The patterns for TSS sites were reproduced in open chromatin regions comprising DHSs and CTCFs.

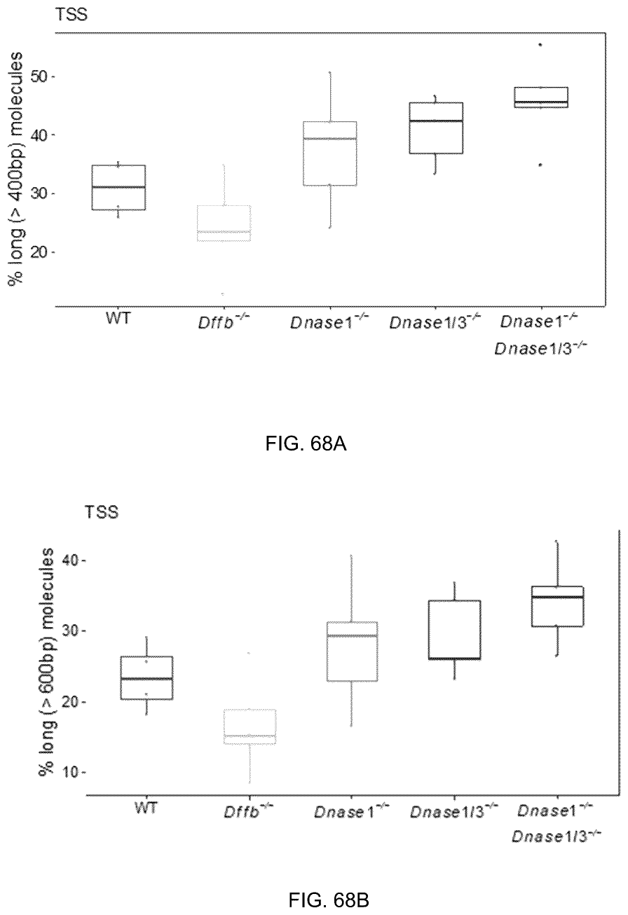

FIG. 68A illustrates boxplots showing proportions of long molecules (>400 bp) originated from 2000 bp upstream and downstream of DHSs and CTCFs for different subjects that lack certain nucleases.

FIG. 68B illustrates boxplots showing proportions of long molecules (>600 bp) originated from 2000 bp upstream and downstream of DHSs and CTCFs for different subjects that lack certain nucleases.

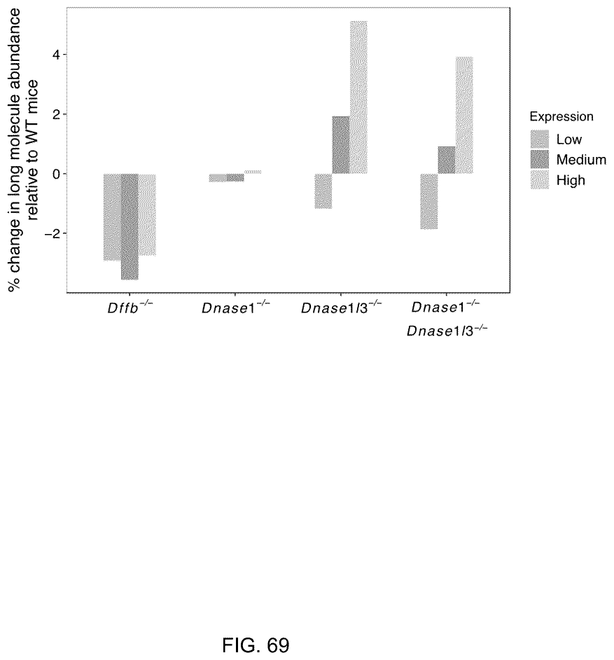

FIG. 69 shows a percent of changes in pooled long molecules (>500 bp) abundance relative to the wild-type mice on low, medium, and high expression gene groups. The highly expressed genes tended to harbor more long molecules in Dffb-competent mice. However, such enrichment of long molecules was absent in Dffb-deficient mice.

### B. Method

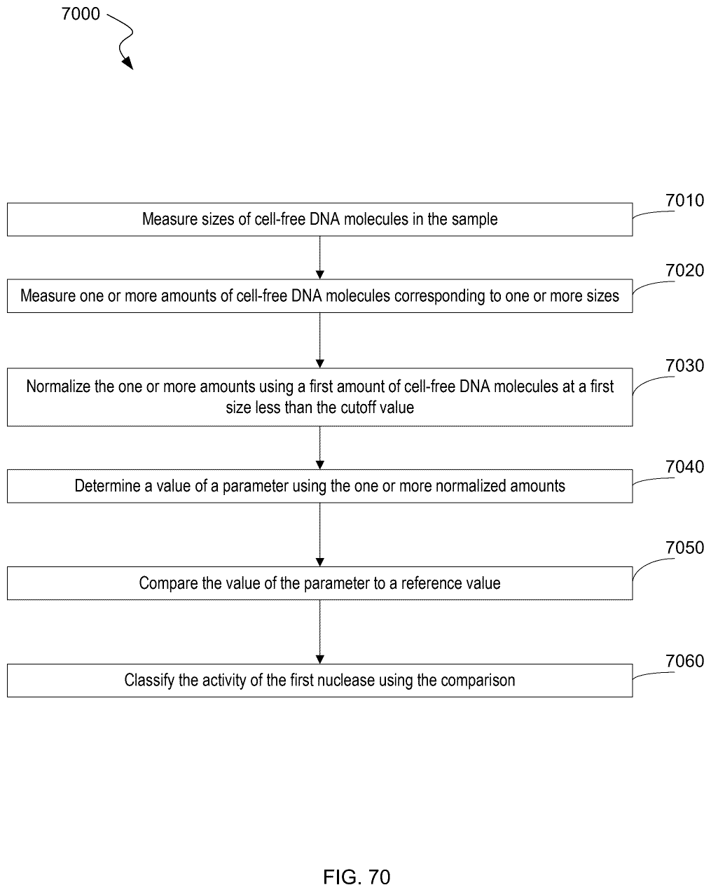

FIG. 70 is a flowchart of an example process 7000 for measuring an activity of a first nuclease in a biological sample uses sizes of cfDNA fragments. The cfDNA fragments may be in open chromatin regions. Example process 7000 may classify an activity of a first nuclease in a biological sample. In some implementations, one or more process blocks of FIG. 70 may be performed by any system described herein. As shown above, when a gene for a nuclease is knocked out, there may be different levels of long cfDNA present. Figures above show that Dffb^{-/-} has the least long (>500 bp) cfDNA molecules; other knockout genes also change the amount of long cfDNA molecules over wild-type.

At block 7010, sizes of cell-free DNA molecules in the biological sample are measured. The sizes can be measured in various ways, as can also be for other techniques described herein. For example, measuring the sizes of cell-free DNA molecules in the biological sample can include performing a physical separation of cell-free DNA molecules having different sizes. Examples of such a physical separation include electrophoresis or filtration.

In some embodiments, the cell-free DNA molecules are located in a set of open chromatin regions (OCRs). For example, the set of OCRs can include transcription start sites (TSSs), DNase1 hypersensitive sites (DHSs), CCCTC-binding factor (CTCF) sites, or a combination thereof. Such cfDNA molecules can be identified by aligning sequence reads of the cell-free DNA molecules to a reference genome to determine genomic coordinates of ends of the cell-free DNA molecules. The genomic coordinates can be used to determine the cell-free DNA molecules that are located in the set of open chromatin regions (OCRs). The sequences reads (e.g., if single molecule sequencing is used) and/or the genomic coordinates can be used to determine the sizes of the cell-free DNA molecules. Various number of OCRs can be used, e.g., at least 10, 20, 50, 100, 1,000, 2,000, 5,000, 10,000, 20,000, 50,000, or 100,000, for this technique and other techniques herein.

At block 7020, one or more amounts of cell-free DNA molecules corresponding to one or more sizes are measured. The one or more sizes can be greater than a cutoff value. The cutoff value may be 400 nt, 500 nt or any cutoff value described herein. The one or more amounts can be combined into a single amount and/or determined as a single among for a size range.

At block 7030, the one or more amounts are normalized using a first amount of cell-free DNA molecules at a first size less than the cutoff value, thereby obtaining one or more normalized amounts. The first size may be a size range. For example, the size may be from 1 to 500 nt.

Normalizing the one or more amounts may be performed in various ways. For example, the normalizing can include dividing the one or more amounts by the first amount. As another example, the first amount can be part of a total amount of cfDNA. Thus, normalizing the one or more amounts can comprise determining a total amount of cell-free DNA molecules, where the total amount includes the first amount. The total amount can then be used to normalize the one or more amounts, e.g., by dividing the one or more amounts (or sum of the one or more amounts) by the total amount.

At block 7040, a value of a parameter is determined using the one or more normalized amounts. The parameter may be a sum of the one or more normalized amounts. In some embodiments, the parameter may be an array of the one or more normalized amounts.

At block 7050, the value of the parameter is compared to a reference value. The reference value is obtained using a sample having known activity levels of a set of nucleases, where the known activity level of a first nuclease is below normal and the known activity level of one or more other nucleases is within a normal range in the sample. The set of nucleases may include Dffb, Dnase1, or Dnase1l3, or a combination thereof. In some embodiments, the reference value may be an array of values, with each value associated with a size of the one or more sizes.

At block 7060, the activity of the first nuclease is classified using the comparison. In embodiments, classifying the activity of the first nuclease may include determining the activity level is below normal or is normal. In embodiments, classifying the activity of the first nuclease may include quantifying the activity level (e.g., in enzyme units). Quantifying the activity level may include interpolating between the activity level at 100% (normal) and 0% (inactive) using the placement of the line or sizes between the two known activity levels.

Process 7000 may include additional implementations, such as any single implementation or any combination of implementations described herein and/or in connection with one or more other processes described elsewhere herein.

Although FIG. 70 shows example blocks of process 7000, in some implementations, process 7000 may include additional blocks, fewer blocks, different blocks, or differently arranged blocks than those depicted in FIG. 70. Additionally, or alternatively, two or more of the blocks of process 7000 may be performed in parallel.

### IX. DISCUSSION

Long cfDNA molecules are found to be preferentially originated from transcriptionally active regions of the genome, with the end frequencies of long cfDNA molecules being more enriched in transcriptional starting sites and open chromatin regions. Long molecules exhibited a distinctive cleavage profile surrounding CpG sites. The ending ratio that makes use of multiple positions surrounding CpG sites can be used to distinguish non-cancer (e.g., non-HCC) individuals from patients with cancer (e.g., HCC). In contrast to a previous report that the generation of short cfDNA molecules might be largely attributed to DNASE1L3, we demonstrate that the characteristic distribution of long cfDNA molecules in the genome might at least in part be attributed to the DFFB enzyme activity using genetically modified mice. This observation suggests that different nucleases involved in the fragmentation of long and short cfDNA molecules in human plasma.

The long molecules uncovered via single-molecule sequencing platforms are likely to be, at least in part, the end product of DFFB other than DNASE1L3, which is supported by the observation of enrichment of A-end fragments in long cfDNA molecules (Yu et al. 2021). Of note, following knocking out of the Dnase1l3 and/or Dnase1 genes in mice, DFFB appears to take on the leading role in cfDNA fragmentation. The higher proportions of long molecules originated from accessible chromatin regions in Dnase1l3^{-/-}. Dnase1^{-/-} and Dnase1l3^{-/-} double knockout mice were reminiscent of the characteristic of long molecules in human plasma. The long cfDNA molecules in human plasma are therefore likely to be the digestion products of DFFB, which have escaped further or secondary digestion of DNASE1L3.

Long cfDNA molecules investigated in this study are more likely to reflect the DFFB activity. The genome-wide representational analysis of long cfDNA molecules provides another means to dissect the DFFB activity and link the enzyme activity to gene transcriptional activity. We have uncovered genomic representation difference between long and short cfDNA molecules in human plasma. The enrichment of long molecules in transcriptionally active regions may have important implications. Notably, the normalized end frequencies at TSSs in long cfDNA molecules were higher than those of short cfDNA molecules, suggesting a potential higher utility for long cfDNA molecules for inferring gene expression when compared with their shorter cfDNA counterparts. Moreover, the abundance of long molecules showed discriminating power in cancer detection.

### X. EXAMPLE IMPLEMENTATIONS

### A. Human Sample Collection

The study was approved by the joint Chinese University of Hong Kong (CUHK)—Hospital Authority New Territories East Cluster clinical research ethics committee. PacBio SMRT sequencing data of plasma cfDNA, including healthy individuals (N=15), pregnancies of different trimesters (N=28), HBV carriers (N=13), and patients with HCC (N=13), were obtained from previous studies (Yu et al. 2021; Choy et al. 2022). Additionally, we collected blood samples from healthy individuals (N=5), HBV carriers (N=6), and patients with HCC (N=35) from the Prince of Wales Hospital, Hong Kong, with written informed consent. Nanopore sequencing data of plasma cfDNA from pregnancies of different trimesters (N=31) were obtained from a prior study (Yu et al. 2023b). Additionally, plasma cfDNA from nonpregnant healthy individuals (N=5) was collected and subjected to ONT sequencing. The blood samples were processed by a double centrifugation protocol (1600 g for 10 min at 4° C. followed by further centrifugation of the plasma at 16,000 g for 10 min at 4° C.) as previously described (Chiu et al. 2001).

### B. Murine Models

Mice with a CRISPR/Cas9-targeted deletion of exon 5 in Dnase1l3 of the C57BL/6 (B6) background were generated by The Jackson Laboratory. Mice carrying a targeted allele mutation of Dnase1 (Dnase1^{tm1.1(KOMP)Vleg}) and mice carrying a targeted allele of Dffb (Dffb^{C57BL/6N-Dffbem1Wtsi}) both on the B6 background were obtained from the Knockout Mouse Project Repository of the University of California, Davis. These mice were obtained under a third-party distribution agreement and are nontransferable. Dnase1l3/mice were cross-bred with Dnase1^{-/-} mice to produce Dnase1^{-/-} Dnase1l3/mice in the Laboratory Animal Services Center of The Chinese University of Hong Kong (CUHK). Mice including wild-type B6 were maintained in the same facility. All experimental procedures were approved by the Animal Experimentation Ethics committee of CUHK and performed in compliance with the Guide for the Care and Use of Laboratory Animals (8^{th} edition) established by the National Institutes of Health.

### C. Mice Sample Collection

Mice were sacrificed and exsanguinated by cardiac puncture. Blood was transferred into EDTA-containing collection tubes. The blood samples were processed by a double centrifugation protocol (1,600×g for 10 min at 4° C., followed by further centrifugation of the plasma at 16,000×g for 10 min at 4° C.) as previously described (Chiu et al. 2001). The resulting plasma was collected.

### D. Library Preparation and Sequencing

Plasma DNA was extracted from 1.2-4 mL of human plasma and 1.1-1.4 mL of pooled mice plasma with the QIAamp circulating nucleic acid kit (Qiagen) according to the manufacturer's instructions. The SMRTbell express template prep kit 2.0 (PacBio) was used for the library preparation of plasma DNA. Briefly, DNA molecules were ligated with hairpin adaptors to form a circularized template. Sequencing primer v4 was annealed to the sequencing template, followed by binding of polymerase to templates using a Sequel II binding kit 2.1 and internal control kit 1.0 (PacBio). SMRT cell 8M was used for sequencing with a Sequel II sequencing 2.0 kit (PacBio), and sequencing movies were collected for 30 h. Nanopore library preparation and sequencing of pregnant samples are as described previously (Yu et al. 2023b). For healthy samples, nanopore libraries were prepared using the native barcoding kit (ONT SQK-NBD114.96) according to the manufacturer's instructions except that a bead-to-sample ratio of three was used in all clean-up steps using AMPure XP beads with prolonged incubation time for DNA repair, end-prep, barcode, and adapter ligation. Short fragment buffer, which retained DNA fragments of all sizes, was used in adapter ligation. Each library was loaded onto a PromethION flow cell R10.4.1 and sequenced on a PromethION device for 72 h.

### E. Sequence Alignment

CCS reads that were generated from at least three PacBio subreads were kept. CCS reads from human and mice data were aligned to hg19 and mm10, respectively, using blasr (Chaisson and Tesler 2012). Nanopore-generated sequences were aligned to hg19 with minimap2 (Li 2018) as described previously (Yu et al. 2023b).

### F. Genomic Representation Analysis

The human genome was partitioned into non-overlapping 100 kb bins. Sequences that mapped to blacklist regions (Amemiya et al. 2019) were removed. Long and short molecules were counted in each bin. The bin counts were smoothed by a window of 1 Mb and normalized by the median bin count of the autosomes. Gene densities were estimated by number of genes in a 100-kb window. The correlations between normalized bin counts and gene densities were calculated using Pearson's method and smoothed data applying 1 Mb-window were used.

To calculate the proportions of long molecules originated from TSSs, DHSs, and CTCFs in mice, we counted long and short molecules overlapped with these regions. A length 2000 bp upstream and downstream of transcription starting sites or peak points of DHSs and CTCFs were regarded as regions of interest. At least 50% size of a molecule needs to be overlapped with these regions of interest to be counted. The proportions long molecules were computed as the number of long molecules divided by the total number of molecules of overlapped with the regions of interest.

### G. Genic Abundance Analysis

To quantify molecule abundance over genes, we adopted similar quantification of transcripts using reads per kilobase per million mapped reads. We counted molecules overlapped with gene bodies and normalized by the total number of molecules and the total length of the genes. For both long and short molecules, we required at least 50% size of a molecule to be overlapped with a gene to be counted. To calculate the Pearson correlation between molecule abundance and gene expression, we used the median molecule abundance of samples and correlated with the median gene expression of each gene set. The Mann-Kendall trend test was performed using the median molecule abundance of samples.

Single cell gene expression data were downloaded from The Human Protein Atlas and The Tabular Muris project for human and mouse, respectively. In human analysis, housekeeping genes from were retrieved from (Eisenberg and Levanon 2013), and 3510 autosomal housekeeping gene were used. A total of 2154 genes with a mean of less than 1 normalized transcript per million across 54 tissues were defined as unexpressed genes. Autosomal protein coding genes were stratified by median expression across 79 cell types. Gene groups of EXP1-5 correspond to genes with median normalized transcript per million in the range of [0, 0.001), [0.001, 1), [1, 8), [8, 30), [30, 11800), with each containing 4836, 3163, 4187, 4816, and 3088 genes, respectively. To correlate the abundance of molecules with gene expression at relatively high resolution in human samples, median expression level of a gene across cell types were log-transformed and scaled to a score ranging from 0 to 1000. Median molecule abundance of genes at each scaled expression level was used. Genes with expression scores of lower and upper quantiles (outliers) were discarded from the correlation analysis.

Murine genes were stratified by median expression across 20 tissue types. Low, medium, and high expression gene groups in mice correspond to autosomal protein coding genes with median normalized transcript count in the range of [0, 50), [50,180) and [180,450), with each containing 10987, 3883, and 5195 genes, respectively. The percentage of change in long molecule abundance between the knockout and wild-type mice was computed using the difference of long molecule abundance between knockout and wild-type mice and divided by the abundance of long molecules in the wild-type mice.

### H. End Frequency Analysis

End frequency analysis was performed surrounding the defined region of interests. The region of interests, including CpGs, DHSs, CTCFs, and TSSs, were extracted as follows.

Transcription starting sites were retrieved using NCBI RefSeq data. DHSs and CTCFs were identified from publicly available data. Tissue-invariant DHSs from the study (Meuleman et al. 2020) were fetched and genomic coordinates were converted from hg38 to hg19 using the UCSC LiftOver tool. 44,997 tissue-invariant DHSs were used in the analysis. 157,556 CTCFs from the ENCODE uniformly processed transcription factor binding stie clusters in human were used for analysis (Dunham et al. 2012). A total of 107,227 DHSs (ENCFF855RCO) and 62,461 CTCFs (ENCFF883UPM) from CH12.LX mouse cell line available via ENCODE were used in mice analysis.

The peak or mid-point of the region of interests was regarded as position 0. The end frequencies within +/−2000 bp were aggregated and normalized with median count of the region. For regulatory regions (DHS and CTCF) and TSS, we applied a 10 bp and 20 bp window to smooth the count. Fold change of end frequencies around DHSs was measured as follows. End frequencies within a DHS region (a median size of 211 bp) were aggregated. In parallel, end frequencies of the equivalent region that are 3000 bp away upstream and downstream were aggregated and used as a normalization factor. Fold change of end frequencies around TSSs was measured using ratio between long and short in human. Normalized end frequencies from position −50 to 50 were aggregated to compute the ratio.

### I. Cleavage Profiles Surrounding CpGs

For human data analysis, commonly methylated and unmethylated CpG sites were defined as methylation level >70% and <30% respectively in human buffy coat, liver, and placental tissues. Whole genome bisulfite sequencing (WGBS) subjected to the Illumina platform from the three tissues available from a previous study (Sun et al. 2015) were used to identify 2,422,965 commonly methylated and 558,267 commonly unmethylated CpGs. For analysis of all CpGs, 26,752,699 autosomal CpGs from the human genome hg19 were obtained. The cleavage proportion to measure the cutting frequency were measured as the number of molecules ends at a particular site divided by sequencing depth at the site, as described in (Zhou et al. 2022). The CGN/NCG motif ratio was calculated using the number of molecules ends at position 0 divided by the number of molecules ends at position −1. The ending ratio (also referred to as a cleavage ratio) was calculated in various ways using sums at various positions, e.g., using aggregated number of molecules ends at position −4, −2, 1, and 4 divided by the number of molecules ends at position −1.

### J. Statistical Analysis

The Mann-Kendall trend test was used for testing monotonic trend in molecule abundance over gene expression groups. Alternative hypothesis that monotonic increasing trend is present was assumed. The Pearson's correlation test was used to measure relations between abundance of molecules and gene expression. The Wilcoxon rank-sum test was used to compare pairwise groups at a significance level of 0.05. The Kruskal-Wallis test, or Friedman test in case of dependent groups, was used to compare three or more groups at a significance level of 0.05. Post hoc pairwise Wilcoxon tests were performed with Benjamini-Hochberg adjustment to yield pairwise P-values.

### XI. TREATMENTS

### A. Further Screening Modalities

Based on any classification, e.g., regarding a pathology or fractional concentration of clinically-relevant DNA, the subject can be referred for additional screening modalities, e.g. using chest X ray, ultrasound, computed tomography, magnetic resonance imaging, or positron emission tomography. Such screening may be performed for cancer.

### B. Treatment Selection

Embodiments of the present disclosure can accurately predict disease relapse, thereby facilitating early intervention and selection of appropriate treatments to improve disease outcome and overall survival rates of subjects. For example, an intensified chemotherapy can be selected for subjects, in the event their corresponding samples are predictive of disease relapse. In another example, a biological sample of a subject who had completed an initial treatment can be sequenced to identify viral DNA that is predictive of disease relapse. In such example, alternative treatment regimen (e.g., a higher dose) and/or a different treatment can be selected for the subject, as the subject's cancer may have been resistant to the initial treatment.

The embodiments may also include treating the subject in response to determining a classification of relapse of the pathology. For example, if the prediction corresponds to a loco-regional failure, surgery can be selected as a possible treatment. In another example, if the prediction corresponds to a distant metastasis, chemotherapy can be additionally selected as a possible treatment. In some embodiments, the treatment includes surgery, radiation therapy, chemotherapy, immunotherapy, targeted therapy, hormone therapy, stem cell therapy, or precision medicine. Based on the determined classification of relapse, a treatment plan can be developed to decrease the risk of harm to the subject and increase overall survival rate. Embodiments may further include treating the subject according to the treatment plan.

### C. Types of Treatments

Embodiments may further include treating the pathology in the patient after determining a classification for the subject. Treatment can be provided according to a determined level of pathology, the fractional concentration of clinically-relevant DNA, or a tissue of origin. For example, an identified mutation can be targeted with a particular drug or chemotherapy. The tissue of origin can be used to guide a surgery or any other form of treatment. And, the level of the pathology can be used to determine how aggressive to be with any type of treatment, which may also be determined based on the level of pathology. A pathology (e.g., cancer) may be treated by chemotherapy, drugs, diet, therapy, and/or surgery. In some embodiments, the more the value of a parameter (e.g., amount or size) exceeds the reference value, the more aggressive the treatment may be.

Treatment may include resection. For bladder cancer, treatments may include transurethral bladder tumor resection (TURBT). This procedure is used for diagnosis, staging and treatment. During TURBT, a surgeon inserts a cystoscope through the urethra into the bladder. The tumor is then removed using a tool with a small wire loop, a laser, or high-energy electricity. For patients with non-muscle invasive bladder cancer (NMIBC), TURBT may be used for treating or eliminating the cancer. Another treatment may include radical cystectomy and lymph node dissection. Radical cystectomy is the removal of the whole bladder and possibly surrounding tissues and organs. Treatment may also include urinary diversion. Urinary diversion is when a physician creates a new path for urine to pass out of the body when the bladder is removed as part of treatment.

Treatment may include chemotherapy, which is the use of drugs to destroy cancer cells, usually by keeping the cancer cells from growing and dividing. The drugs may involve, for example but are not limited to, mitomycin-C (available as a generic drug), gemcitabine (Gemzar), and thiotepa (Tepadina) for intravesical chemotherapy. The systemic chemotherapy may involve, for example but not limited to, cisplatin gemcitabine, methotrexate (Rheumatrex, Trexall), vinblastine (Velban), doxorubicin, and cisplatin.

In some embodiments, treatment may include immunotherapy. Immunotherapy may include immune checkpoint inhibitors that block a protein called PD-1. Inhibitors may include but are not limited to atezolizumab (Tecentriq), nivolumab (Opdivo), avelumab (Bavencio), durvalumab (Imfinzi), and pembrolizumab (Keytruda).

Treatment embodiments may also include targeted therapy. Targeted therapy is a treatment that targets the cancer's specific genes and/or proteins that contributes to cancer growth and survival. For example, erdafitinib is a drug given orally that is approved to treat people with locally advanced or metastatic urothelial carcinoma with FGFR3 or FGFR2 genetic mutations that has continued to grow or spread of cancer cells.

Some treatments may include radiation therapy. Radiation therapy is the use of high-energy x-rays or other particles to destroy cancer cells. In addition to each individual treatment, combinations of these treatments described herein may be used. In some embodiments, when the value of the parameter exceeds a threshold value, which itself exceeds a reference value, a combination of the treatments may be used. Information on treatments in the references are incorporated herein by reference.

### XII. EXAMPLE SYSTEMS

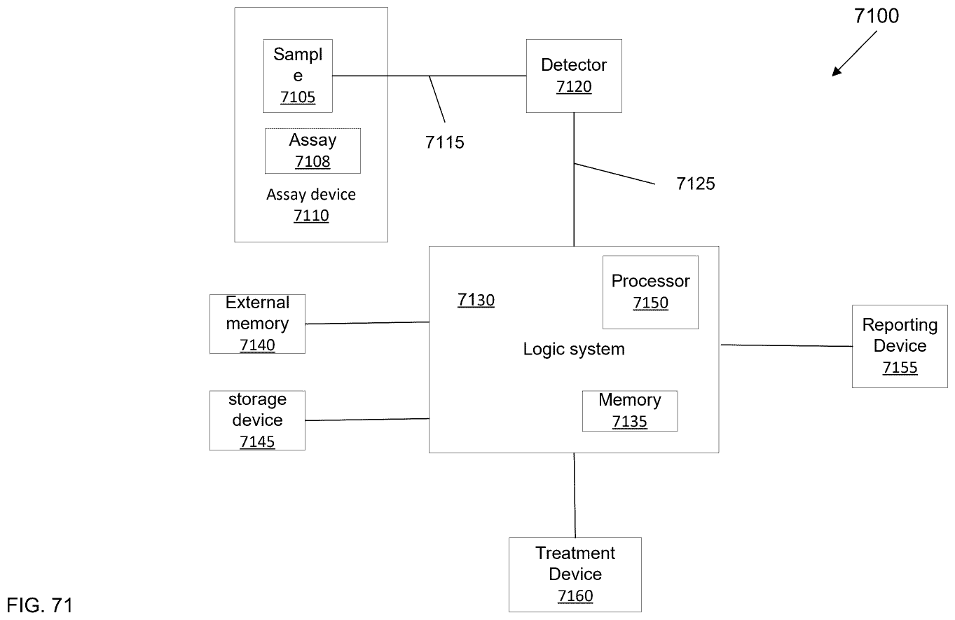

FIG. 71 illustrates a measurement system 7100 according to an embodiment of the present disclosure. The system as shown includes a sample 7105, such as cell-free nucleic acid molecules (e.g., DNA and/or RNA) within an assay device 7110, where an assay 7108 can be performed on sample 7105. For example, sample 7105 can be contacted with reagents of assay 7108 to provide a signal (e.g., an intensity signal) of a physical characteristic 7115 (e.g., sequence information of a cell-free nucleic acid molecule). An example of an assay device can be a flow cell that includes probes and/or primers of an assay or a tube through which a droplet moves (with the droplet including the assay). Physical characteristic 7115 (e.g., a fluorescence intensity, a voltage, or a current), from the sample is detected by detector 7120. Detector 7120 can take a measurement at intervals (e.g., periodic intervals) to obtain data points that make up a data signal. In one embodiment, an analog-to-digital converter converts an analog signal from the detector into digital form at a plurality of times.

Assay device 7110 and detector 7120 can form an assay system, e.g., a PCR system or a sequencing system that performs sequencing according to embodiments described herein. A data signal 7125 is sent from detector 7120 to logic system 7130. As an example, data signal 7125 can be used to determine sequences and/or locations in a reference genome of nucleic acid molecules (e.g., DNA and/or RNA). Data signal 7125 can include various measurements made at a same time, e.g., different colors of fluorescent dyes or different electrical signals for different molecule of sample 7105, and thus data signal 7125 can correspond to multiple signals. Data signal 7125 may be stored in a local memory 7135, an external memory 7140, or a storage device 7145. The assay system can be comprised of multiple assay devices and detectors.

Logic system 7130 may be, or may include, a computer system, ASIC, microprocessor, graphics processing unit (GPU), etc. It may also include or be coupled with a display (e.g., monitor, LED display, etc.) and a user input device (e.g., mouse, keyboard, buttons, etc.). Logic system 7130 and the other components may be part of a stand-alone or network connected computer system, or they may be directly attached to or incorporated in a device (e.g., a sequencing device) that includes detector 7120 and/or assay device 7110. Logic system 7130 may also include software that executes in a processor 7150. Logic system 7130 may include a computer readable medium storing instructions for controlling measurement system 7100 to perform any of the methods described herein. For example, logic system 7130 can provide commands to a system that includes assay device 7110 such that sequencing or other physical operations are performed. Such physical operations can be performed in a particular order, e.g., with reagents being added and removed in a particular order. Such physical operations may be performed by a robotics system, e.g., including a robotic arm, as may be used to obtain a sample and perform an assay. Logic system 7130 can perform any steps of methods described herein that perform computer processing.

Measurement system 7100 may also include a treatment device 7160, which can provide a treatment to the subject. Treatment device 7160 can determine a treatment and/or be used to perform a treatment. Examples of such treatment can include surgery, radiation therapy, chemotherapy, immunotherapy, targeted therapy, hormone therapy, and stem cell transplant. Logic system 7130 may be connected to treatment device 7160, e.g., to provide results of a method described herein. The treatment device may receive inputs from other devices, such as an imaging device and user inputs (e.g., to control the treatment, such as controls over a robotic system).

Measurement system 7100 may also include a reporting device 7155, which can present results of any of the methods describe herein, e.g., as determined using the measurement system. Reporting device 7155 can be in communication with a reporting module within logic system 7130 that can aggregate, format, and send a report to reporting device 7155. The reporting module can present information determined using any of the method described herein. The information can be presented by reporting device 7155 in any format that can be recognized and interpreted by a user of the measurement system 7100. For example, the information can be presented by reporting device 7155 in a displayed, printed, or transmitted format, or any combination thereof.

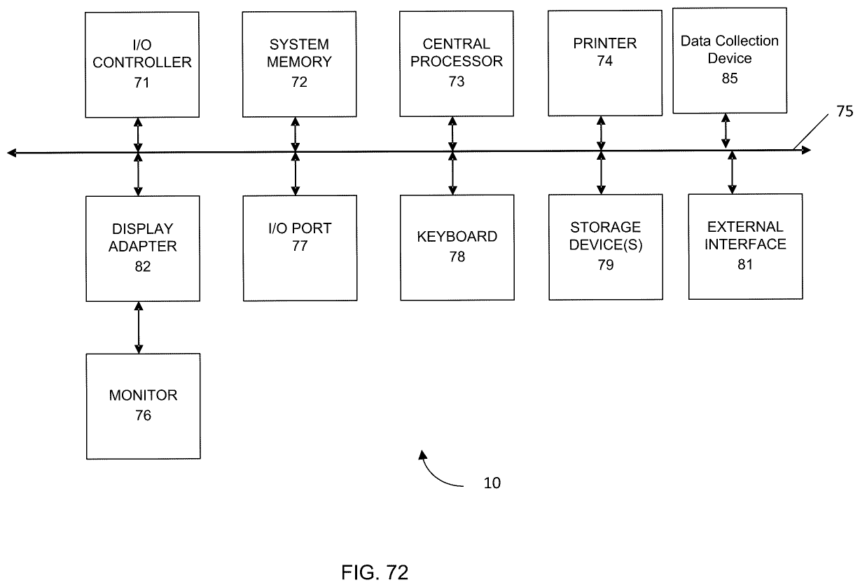

Any of the computer systems mentioned herein may utilize any suitable number of subsystems. Examples of such subsystems are shown in FIG. 72 in computer system 10. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems shown in FIG. 72 are interconnected via a system bus 75. Additional subsystems such as a printer 74, keyboard 78, storage device(s) 79, monitor 76 (e.g., a display screen, such as an LED), which is coupled to display adapter 82, and others are shown. Peripherals and input/output (I/O) devices, which couple to I/O controller 71, can be connected to the computer system by any number of means known in the art such as input/output (I/O) port 77 (e.g., USB, FireWire®). For example, I/O port 77 or external interface 81 (e.g., Ethernet, Wi-Fi, etc.) can be used to connect computer system 10 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components. In various embodiments, methods may involve various numbers of clients and/or servers, including at least 10, 20, 50, 100, 200, 500, 1,000, or 10,000 devices. Methods can include various numbers of communication messages between devices, including at least 100, 200, 500, 1,000, 10,000, 50,000, 100,000, 500,00, or one million communication messages. Such communications can involve at least 1 MB, 10 MB, 100 MB, 1 GB, 10 GB, or 100 GB of data.

Aspects of embodiments can be implemented in the form of control logic using hardware circuitry (e.g., an application specific integrated circuit or field programmable gate array) and/or using computer software stored in a memory with a generally programmable processor in a modular or integrated manner, and thus a processor can include memory storing software instructions that configure hardware circuitry, as well as an FPGA with configuration instructions or an ASIC. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present disclosure using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk) or Blu-ray disk, flash memory, and the like. The computer readable medium may be any combination of such devices. In addition, the order of operations may be re-arranged. A process can be terminated when its operations are completed but could have additional steps not included in a figure. A process may correspond to a method, a function, a procedure, a subroutine, a subprogram, etc. When a process corresponds to a function, its termination may correspond to a return of the function to the calling function or the main function.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device (e.g., as firmware) or provided separately from other devices (e.g., via Internet download). Any such computer readable medium may reside on or within a single computer product (e.g., a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Any operations performed with a processor (e.g., aligning, determining, comparing, computing, calculating) may be performed in real-time. The term “real-time” may refer to computing operations or processes that are completed within a certain time constraint. As examples, a time constraint may be 30 seconds, 1 minute, 10 minutes, 30 minutes, 1 hour, 4 hours, 1 day, or 7 days. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order. Additionally, portions of these steps may be used with portions of other steps from other methods. Also, all or portions of a step may be optional. Additionally, any of the steps of any of the methods can be performed with modules, units, circuits, or other means of a system for performing these steps.

The specific details of particular embodiments may be combined in any suitable manner without departing from the spirit and scope of embodiments of the disclosure. However, other embodiments of the disclosure may be directed to specific embodiments relating to each individual aspect, or specific combinations of these individual aspects.

The above description of example embodiments of the present disclosure has been presented for the purposes of illustration and description. It is not intended to be exhaustive or to limit the disclosure to the precise form described, and many modifications and variations are possible in light of the teaching above.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

The claims may be drafted to exclude any element which may be optional. As such, this statement is intended to serve as antecedent basis for use of such exclusive terminology as “solely”, “only”, and the like in connection with the recitation of claim elements, or the use of a “negative” limitation.

All patents, patent applications, publications, and descriptions mentioned herein are incorporated by reference in their entirety for all purposes. None is admitted as prior art. Where a conflict exists between the instant application and a reference provided herein, the instant application shall dominate.

### XIII. REFERENCES

Amemiya H M, Kundaje A, Boyle A P. 2019. The ENCODE Blacklist: Identification of Problematic Regions of the Genome. Sci Rep 9: 9354.
BAC Resource Consortium T, Cheung V G, Nowak N, Jang W, Kirsch I R, Zhao S, Chen X-N, Furey T S, Kim U-J, Kuo W-L, et al. 2001. Integration of cytogenetic landmarks into the draft sequence of the human genome. Nature 409: 953-958.
Bickmore W A, Sumner A T. 1989. Mammalian chromosome banding an expression of genome organization. Trends in Genetics 5: 144-148.
Chan K C A, Zhang J, Hui A B Y, Wong N, Lau T K, Leung T N, Lo K-W, Huang D W S, Lo Y M D. 2004. Size Distributions of Maternal and Fetal DNA in Maternal Plasma. Clinical Chemistry 50: 88-92.
Chan R W Y, Serpas L, Ni M, Volpi S, Hiraki L T, Tam L-S, Rashidfarrokhi A, Wong P C H, Tam L H P, Wang Y, et al. 2020. Plasma DNA Profile Associated with DNASE1L3 Gene Mutations: Clinical Observations, Relationships to Nuclease Substrate Preference, and In Vivo Correction. The American Journal of Human Genetics 107: 882-894.
Chiu R W K, Poon L L M, Lau T K, Leung T N, Wong E M C, Lo Y M D. 2001. Effects of Blood-Processing Protocols on Fetal and Total DNA Quantification in Maternal Plasma. Clinical Chemistry 47: 1607-1613.
Choy L Y L, Peng W, Jiang P, Cheng S H, Yu S C Y, Shang H, Olivia Tse O Y, Wong J, Wong V W S, Wong G L H, et al. 2022. Single-Molecule Sequencing Enables Long Cell-Free DNA Detection and Direct Methylation Analysis for Cancer Patients. Clinical Chemistry 68: 1151-1163.
Dunham I, Kundaje A, Aldred S F, Collins P J, Davis C A, Doyle F, Epstein C B, Frietze S, Harrow J, Kaul R, et al. 2012. An integrated encyclopedia of DNA elements in the human genome. Nature 489: 57-74.
Eisenberg E, Levanon E Y. 2013. Human housekeeping genes, revisited. Trends in Genetics 29: 569-574.
Grimwood J, Gordon L A, Olsen A, Terry A, Schmutz J, Lamerdin J, Hellsten U, Goodstein D, Couronne O, Tran-Gyamfi M, et al. 2004. The DNA sequence and biology of human chromosome 19. Nature 428: 529-535.
Han D S C, Ni M, Chan R W Y, Chan V W H, Lui K O, Chiu R W K, Lo Y M D. 2020. The Biology of Cell-free DNA Fragmentation and the Roles of DNASE1, DNASE1L3, and DFFB. The American Journal of Human Genetics 106: 202-214.
Han D S C, Ni M, Chan R W Y, Wong D K L, Hiraki L T, Volpi S, Jiang P, Lui K O, Chan K C A, Chiu R W K, et al. 2021. Nuclease deficiencies alter plasma cell-free DNA methylation profiles. Genome Res 31: 2008-2021.
Ivanov M, Baranova A, Butler T, Spellman P, Mileyko V. 2015. Non-random fragmentation patterns in circulating cell-free DNA reflect epigenetic regulation. BMC Genomics 16: S1.
Jiang P, Chan C W M, Chan K C A, Cheng S H, Wong J, Wong V W-S, Wong G L H, Chan S L, Mok T S K, Chan H L Y, et al. 2015. Lengthening and shortening of plasma DNA in hepatocellular carcinoma patients. PNAS 112: E1317-E1325.
Katsman E, Orlanski S, Martignano F, Fox-Fisher I, Shemer R, Dor Y, Zick A, Eden A, Petrini I, Conticello S G, et al. 2022. Detecting cell-of-origin and cancer-specific methylation features of cell-free DNA from Nanopore sequencing. Genome Biology 23: 158.
Larsen B D, Sorensen C S. 2017. The caspase-activated DNase: apoptosis and beyond. The FEBS Journal 284: 1160-1170.
Lau B T, Almeda A, Schauer M, McNamara M, Bai X, Meng Q, Partha M, Grimes S M, Lee H, Heestand G M, et al. 2023. Single-molecule methylation profiles of cell-free DNA in cancer with nanopore sequencing. Genome Medicine 15: 33.
Lensing S V, Marsico G, Hansel-Hertsch R, Lam E Y, Tannahill D, Balasubramanian S. 2016. DSBCapture: in situ capture and sequencing of DNA breaks. Nat Methods 13: 855-857.
Lo Y M D, Chan K C A, Sun H, Chen E Z, Jiang P, Lun F M F, Zheng Y W, Leung T Y, Lau T K, Cantor C R, et al. 2010. Maternal Plasma DNA Sequencing Reveals the Genome-Wide Genetic and Mutational Profile of the Fetus. Science Translational Medicine 2: 61ra91-61ra91.
Lo Y M D, Han D S C, Jiang P, Chiu R W K. 2021. Epigenetics, fragmentomics, and topology of cell-free DNA in liquid biopsies. Science 372. http://science.sciencemag.org/content/372/6538/eaaw3616 (Accessed Apr. 12, 2021).
Meuleman W, Muratov A, Rynes E, Halow J, Lee K, Bates D, Diegel M, Dunn D, Neri F, Teodosiadis A, et al. 2020. Index and biological spectrum of human DNase I hypersensitive sites. Nature 584: 244-251.
Mouliere F, Chandrananda D, Piskorz A M, Moore E K, Morris J, Ahlborn L B, Mair R, Goranova T, Marass F, Heider K, et al. 2018. Enhanced detection of circulating tumor DNA by fragment size analysis. Science Translational Medicine 10. https://stm.sciencemag.org/content/10/466/eaat4921 (Accessed Sep. 7, 2020).
Nagata S, Nagase H, Kawane K, Mukae N, Fukuyama H. 2003. Degradation of chromosomal DNA during apoptosis. Cell Death Differ 10: 108-116.
Serpas L, Chan R W Y, Jiang P, Ni M, Sun K, Rashidfarrokhi A, Soni C, Sisirak V, Lee W-S, Cheng S H, et al. 2019. Dnase1l3 deletion causes aberrations in length and end-motif frequencies in plasma DNA. PNAS 116: 641-649.
Snyder M W, Kircher M, Hill A J, Daza R M, Shendure J. 2016. Cell-free DNA Comprises an In Vivo Nucleosome Footprint that Informs Its Tissues-Of-Origin. Cell 164: 57-68.
Sun K, Jiang P, Chan K C A, Wong J, Cheng Y K Y, Liang R H S, Chan W, Ma E S K, Chan S L, Cheng S H, et al. 2015. Plasma DNA tissue mapping by genome-wide methylation sequencing for noninvasive prenatal, cancer, and transplantation assessments. PNAS 112: E5503-E5512.
Sun K, Jiang P, Cheng S H, Cheng T H T, Wong J, Wong V W S, Ng S S M, Ma B B Y, Leung T Y, Chan S L, et al. 2019. Orientation-aware plasma cell-free DNA fragmentation analysis in open chromatin regions informs tissue of origin. Genome Res 29: 418-427.
Toné S, Sugimoto K, Tanda K, Suda T, Uehira K, Kanouchi H, Samejima K, Minatogawa Y, Earnshaw W C. 2007. Three distinct stages of apoptotic nuclear condensation revealed by time-lapse imaging, biochemical and electron microscopy analysis of cell-free apoptosis. Experimental Cell Research 313: 3635-3644.
Tse O Y O, Jiang P, Cheng S H, Peng W, Shang H, Wong J, Chan S L, Poon L C Y, Leung T Y, Chan K C A, et al. 2021. Genome-wide detection of cytosine methylation by single molecule real-time sequencing. Proceedings of the National Academy of Sciences 118: e2019768118.
Ulz P, Perakis S, Zhou Q, Moser T, Belic J, Lazzeri I, Wölfler A, Zebisch A, Gerger A, Pristauz G, et al. 2019. Inference of transcription factor binding from cell-free DNA enables tumor subtype prediction and early detection. Nature Communications 10: 4666.
Ulz P, Thallinger G G, Auer M, Graf R, Kashofer K, Jahn S W, Abete L, Pristauz G, Petru E, Geigl J B, et al. 2016. Inferring expressed genes by whole-genome sequencing of plasma DNA. Nature Genetics 48: 1273.
Yu S C Y, Chan K C A, Zheng Y W L, Jiang P, Liao G J W, Sun H, Akolekar R, Leung T Y, Go A T J I, Vugt J M G van, et al. 2014. Size-based molecular diagnostics using plasma DNA for noninvasive prenatal testing. PNAS 111: 8583-8588.
Yu S C Y, Choy L Y L, Lo Y M D. 2023a. ‘Longing’ for the Next Generation of Liquid Biopsy: The Diagnostic Potential of Long Cell-Free DNA in Oncology and Prenatal Testing. Mol Diagn Ther. https://doi.org/10.1007/s40291-023-00661-2 (Accessed Aug. 15, 2023).
Yu S C Y, Deng J, Qiao R, Cheng S H, Peng W, Lau S L, Choy L Y L, Leung T Y, Wong J, Wong V W-S, et al. 2023b. Comparison of Single Molecule, Real-Time Sequencing and Nanopore Sequencing for Analysis of the Size, End-Motif, and Tissue-of-Origin of Long Cell-Free DNA in Plasma. Clinical Chemistry 69: 168-179.
Yu S C Y, Jiang P, Peng W, Cheng S H, Cheung Y T T, Tse O Y O, Shang H, Poon L C, Leung T Y, Chan K C A, et al. 2021. Single-molecule sequencing reveals a large population of long cell-free DNA molecules in maternal plasma. Proceedings of the National Academy of Sciences 118: e2114937118.
Zhou Q, Kang G, Jiang P, Qiao R, Lam W K J, Yu S C Y, Ma M-J L, Ji L, Cheng S H, Gai W, et al. 2022. Epigenetic analysis of cell-free DNA by fragmentomic profiling. Proceedings of the National Academy of Sciences 119: e2209852119.
Zhou Z, Ma M-J L, Chan R W Y, Lam W K J, Peng W, Gai W, Hu X, Ding S C, Ji L, Zhou Q, et al. 2023. Fragmentation landscape of cell-free DNA revealed by deconvolutional analysis of end motifs. Proceedings of the National Academy of Sciences 120: e2220982120.
Ziller M J, Gu H, Müller F, Donaghey J, Tsai L T-Y, Kohlbacher O, De Jager P L, Rosen E D, Bennett D A, Bernstein B E, et al. 2013. Charting a dynamic DNA methylation landscape of the human genome. Nature 500: 477-481.

What is claimed is:

1. A method of detecting cancer, the method comprising:
aligning sequence reads of cell-free DNA molecules from a biological sample of a subject to a reference genome to determine genomic coordinates of ends of the cell-free DNA molecules, wherein the biological sample is blood, plasma, or serum;
measuring a proportion of a group of the cell-free DNA molecules within an open chromatin region (OCR) of a set of OCRs;
determining a value of a parameter using the proportion;
comparing the value of the parameter to a reference value; and
determining, using the comparison, a classification of cancer in the subject.

2. The method of claim 1, wherein a cell-free DNA molecule is within an OCR when an end is located at one or more positions within a window including a center of the OCR.

3. The method of claim 1, wherein a cell-free DNA molecule is within an OCR when at least a specified percentage of the cell-free DNA molecule overlaps with the OCR.

4. The method of claim 1, wherein the value of the parameter is the proportion.

5. The method of claim 1, wherein measuring the proportion comprises:
measuring a first amount of the group of the cell-free DNA molecules having a size greater than a first cutoff value;
measuring a second amount of the group of the cell-free DNA molecules having a size equal to or less than a second cutoff value, wherein the second cutoff value is equal to or less than the first cutoff value; and
determining the proportion using the first amount and the second amount, wherein the proportion reflects a relative amount between the first amount and the second amount.

6. The method of claim 5, wherein the first cutoff value is at least 400 nt.

7. The method of claim 5, wherein the second cutoff value is 500 nt or less.

8. The method of claim 1, wherein measuring the proportion comprises:
measuring a first amount of the group of the cell-free DNA molecules; and
generating the proportion by normalizing the first amount using a second amount of the cell-free DNA molecules that are outside the set of OCRs.

9. The method of claim 8, wherein normalizing the first amount uses a total amount of the sequence reads aligned to the reference genome.

10. The method of claim 8, wherein the group of the cell-free DNA molecules have a size greater than a first cutoff value, the method further comprising:
measuring another proportion of another group of the cell-free DNA molecules within an OCR of the set of OCRs, wherein the other group of cell-free DNA molecules have sizes equal to or less than a second cutoff value, wherein the second cutoff value is equal to or less than the first cutoff value,
wherein determining the value of the parameter uses the proportion and the other proportion.

11. The method of claim 1, wherein each of the set of OCRs is associated with a first tissue type, and wherein the cancer is of the first tissue type.

12. The method of claim 1, wherein the group of the cell-free DNA molecules have a size greater than a cutoff value.

13. The method of claim 1, wherein the group of the cell-free DNA molecules have a size less than a cutoff value.

14. The method of claim 1, wherein the set of OCRs includes transcription start sites (TSSs), DNase1 hypersensitive sites (DHSs), CCCTC-binding factor (CTCF) sites, or a combination thereof.

15. The method of claim 1, wherein the reference value is determined using training samples from subjects having known classifications of cancer.

16. A method of estimating an expression level of a gene, the method comprising:
measuring sizes of cell-free DNA molecules;
identifying a group of cell-free DNA molecules corresponding to locations in a genomic region within the gene;
measuring a first amount of the group of cell-free DNA molecules having a size greater than a first cutoff value;
measuring a second amount of the group of cell-free DNA molecules having a size less than a second cutoff value that is equal to or less than the first cutoff value;
determining a value of a parameter using the first amount and the second amount, wherein the parameter reflects a relative amount between the first amount and the second amount;
comparing the value of the parameter to one or more calibration values, wherein the one or more calibration values are determined from samples with known expression levels of the gene; and
estimating the expression level for the gene using the comparison.

17. The method of claim 16, wherein identifying the group of cell-free DNA molecules comprises:
aligning sequence reads of the group of cell-free DNA molecules to a reference genome to determine genomic coordinates of ends of the group of cell-free DNA molecules; and
determining, using the genomic coordinates, the group of cell-free DNA molecules are located in the genomic region.

18. The method of claim 17, wherein measuring the sizes of cell-free DNA molecules uses the sequence reads.

19. The method of claim 16, wherein the first cutoff value is at least 400 nt.

20. The method of claim 16, wherein the second cutoff value is 500 nt or less.

21. The method of claim 16, wherein identifying the group of cell-free DNA molecules comprises identifying cell-free DNA molecules having an end located at one or more positions within the genomic region with the gene.

22. The method of claim 16, wherein identifying the group of cell-free DNA molecules comprises identifying cell-free DNA molecules having at least a specified percentage of the cell-free DNA molecule overlapping with the gene.

23. The method of claim 22, wherein the specified percentage is 50%, and wherein identifying the group of cell-free DNA molecules comprises identifying cell-free DNA molecules having at least 50% of their length in the genomic region within the gene.

24. The method of claim 16, wherein identifying the group of cell-free DNA molecules comprises identifying cell-free DNA molecules having an end located in the genomic region.

25. The method of claim 16, wherein the expression level is a categorical level of expression.

26. The method of claim 16, wherein:
the gene is expressed by a first tissue, and
the expression level is a tissue-specific expression level.

27. The method of claim 26, further comprising:
comparing the expression level to one or more reference values, wherein the one or more reference values are determined from samples with known fractional concentrations of cell-free DNA molecules from the first tissue, and
estimating the fractional concentration of cell-free DNA molecules from the first tissue using the comparison.

28. A method of detecting cancer, the method comprising:
aligning sequence reads of cell-free DNA molecules from a biological sample of a subject to a reference genome to determine genomic coordinates of ends of the cell-free DNA molecules;
for each CpG site of a plurality of CpG sites:
measuring, using the genomic coordinates, a first amount of cell-free DNA molecules ending at a first position within a window around the CpG site, thereby determining a plurality of first amounts;
measuring, using the genomic coordinates, a second amount of cell-free DNA molecules ending at a second position within the window around the CpG site, thereby determining a plurality of second amounts; and
measuring, using the genomic coordinates, a third amount of cell-free DNA molecules ending at a third position within the window around the CpG site, thereby determining a plurality of third amounts, wherein the plurality of CpG sites comprise one or more hypermethylated CpG sites and one or more hypomethylated CpG sites;
aggregating the plurality of first amounts to obtain a first aggregated amount;
aggregating the plurality of second amounts to obtain a second aggregated amount;
aggregating the plurality of third amounts to obtain a third aggregated amount;
determining a value of a parameter using the first aggregated amount, the second aggregated amount, and the third aggregated amount;
comparing the value of the parameter to a reference value; and
determining, using the comparison, a classification of cancer in the subject.

29. The method of claim 28, wherein the window includes positions −5 to +5 relative to the CpG site.

30. The method of claim 28, wherein the cell-free DNA molecules have sizes above a cutoff value, wherein the cutoff value is at least 400 nt.

31. The method of claim 28, wherein the cell-free DNA molecules have sizes below a cutoff value, wherein the cutoff value is 500 nt or less.

32. The method of claim 28, wherein:
the first position, the second position, and the third position are each at one of positions −4, −2, −1, +1, or +4.

33. The method of claim 28, wherein the parameter includes a normalized sum that is determined by:
determining a sum that includes the first aggregated amount and the second aggregated amount; and
normalizing the sum using the third aggregated amount.

34. The method of claim 33, wherein the normalized sum is a first normalized sum determined for cell-free DNA molecules having sizes above a first cutoff value, wherein the first cutoff value is of at least 400 nt, and wherein the parameter further includes a second normalized sum (1) determined for the first position, the second position, and the third position and (2) determined for cell-free DNA molecules having sizes equal to or less than a second cutoff value, wherein the second cutoff value is equal to or less than the first cutoff value.

35. The method of claim 28, wherein each of the first aggregated amount, the second aggregated amount, and the third aggregated amount are normalized before determining the value of the parameter.

36. The method of claim 28, further comprising:
measuring a fourth amount of cell-free DNA molecules ending at a fourth position within the window around the CpG site, thereby determining a plurality of fourth amounts; and
aggregating the plurality of fourth amounts to obtain a fourth aggregated amount,
wherein:
determining the value of the parameter further uses the fourth aggregated amount,
the value of the parameter is a normalized sum of the first aggregated amount, the second aggregated amount, the third aggregated amount, and the fourth aggregated amount,
the first position is −4,
the second position is −2,
the third position is +1, and
the fourth position is +4.

37. The method of claim 28, wherein the parameter is a normalized parameter.

38. The method of claim 37, wherein determining the value of the parameter comprises:
for each CpG site of the plurality of CpG sites:
measuring a fourth amount of cell-free DNA molecules ending at a fourth position within the window around the CpG site, thereby determining a plurality of fourth amounts;
aggregating the plurality of fourth amounts to obtain a fourth aggregated amount; and
normalizing the first aggregated amount, the second aggregated amount, and the third aggregated amount using the fourth aggregated amount.

39. The method of claim 38, wherein the fourth position is position −1.

40. The method of claim 28, wherein the window includes positions −10 to +10 relative to the CpG site.

41. The method of claim 28, wherein determining the value of the parameter comprises:
inputting the first aggregated amount, the second aggregated amount, and the third aggregated amount into a model, and wherein the model performs the comparing of the value of the parameter to the reference value.

42. The method of claim 41, wherein the cell-free DNA molecules have sizes above a first cutoff value, wherein the first cutoff value is of at least 400 nt, the method further comprising:
measuring a fourth aggregated amount for a group of short cell-free DNA molecules at the first position;
measuring a fifth aggregated amount for the group of short cell-free DNA molecules at the second position; and
measuring a sixth aggregated amount for the group of short cell-free DNA molecules at the third position, wherein the short cell-free DNA molecules have sizes equal to or less than a second cutoff value, wherein the second cutoff value is equal to or less than the first cutoff value.

43. The method of claim 41, wherein the model uses a feature vector comprising aggregated amounts of cell-free DNA molecules ending at at least five positions within the window, and wherein the window consists of 11 positions or less centered around the CpG site.

44. The method of claim 41, wherein the model trained is trained by:
receiving training input data comprising, for each training sample of a plurality of training samples:
for each CpG site of the plurality of CpG sites:
a first training amount of cell-free DNA molecules ending at the first position within the window around the CpG site,
a second training amount of cell-free DNA molecules ending at the second position within the window around the CpG site, and
a third training amount of cell-free DNA molecules ending at the third position within the window around the CpG site,
storing a training set comprising the training input data and a label for each training sample, the label indicating a known classification of cancer of a training subject associated with the training sample, and
optimizing, using the training set, model parameters based on outputs of the model matching or not matching corresponding labels when the training input data is input to the model.

45. The method of claim 28, wherein:
the first amount of cell-free DNA molecules consists of cell-free DNA molecules having a 5′ end at the first position,
the second amount of cell-free DNA molecules consists of cell-free DNA molecules having a 5′ end at the second position, and
the third amount of cell-free DNA molecules consists of cell-free DNA molecules having a 5′ end at the third position.

46. The method of claim 28, wherein the reference value is determined using training samples from subjects having known classifications of cancer.

47. A method of classifying an activity level of a first nuclease in a biological sample, the method comprising:
measuring sizes of cell-free DNA molecules in the biological sample;
measuring one or more amounts of cell-free DNA molecules corresponding to one or more sizes, wherein the one or more sizes are greater than a cutoff value;
normalizing the one or more amounts using a first amount of cell-free DNA molecules at a first size less than the cutoff value, thereby obtaining one or more normalized amounts;
determining a value of a parameter using the one or more normalized amounts;
comparing the value of the parameter to a reference value, the reference value obtained using a sample having known activity levels of a set of nucleases, wherein a known activity level of the first nuclease is below normal and the known activity level of one or more other nucleases is within a normal range in the sample; and
classifying the activity level of the first nuclease using the comparison.

48. The method of claim 47, wherein the cell-free DNA molecules are located in a set of open chromatin regions (OCRs).

49. The method of claim 48, wherein the set of OCRs includes transcription start sites (TSSs), DNase1 hypersensitive sites (DHSs), CCCTC-binding factor (CTCF) sites, or a combination thereof.

50. The method of claim 48, further comprising:
aligning sequence reads of the cell-free DNA molecules to a reference genome to determine genomic coordinates of ends of the cell-free DNA molecules; and
determining, using the genomic coordinates, the cell-free DNA molecules are located in the set of open chromatin regions (OCRs).

51. The method of claim 47, wherein the cutoff value is at least 400 bp.

52. The method of claim 47, wherein the first size is a size range.

53. The method of claim 47, wherein classifying the activity of the first nuclease comprises determining the activity level is below normal.

54. The method of claim 47, wherein classifying the activity of the first nuclease comprises quantifying the activity level.

55. The method of claim 47, wherein the set of nucleases comprises Dffb, Dnase1, or Dnase1l3, or a combination thereof.

56. The method of claim 47, wherein normalizing the one or more amounts comprises dividing the one or more amounts by the first amount.

57. The method of claim 47, wherein normalizing the one or more amounts comprises:
determining a total amount of cell-free DNA molecules, wherein the total amount includes the first amount; and
using the total amount to normalize the one or more amounts.

58. The method of claim 47, wherein the parameter is a sum of the one or more normalized amounts.

59. The method of claim 47, wherein measuring the sizes of cell-free DNA molecules in the biological sample includes performing a physical separation of cell-free DNA molecules having different sizes.

60. A method of identifying an open chromatin region (OCR) in a genome of a subject, the method comprising:
measuring sizes of cell-free DNA molecules in a biological sample of the subject;
aligning sequence reads of cell-free DNA molecules to a reference genome to determine genomic coordinates of an end of each cell-free DNA molecule;
for each position of a plurality of positions in a genomic region:
measuring a first amount of cell-free DNA molecules having a size greater than a first cutoff value and having an end at the position,
measuring a second amount of cell-free DNA molecules having a size less than a second cutoff value and having an end at the position, the second cutoff value being equal to or less than the first cutoff value;
determining a value of a parameter using the first amount and the second amount, wherein the parameter reflects a relative amount between the first amount and the second amount, and
comparing the value of the parameter to a reference value to identify a set of positions where the value of the parameter exceeds the reference value; and
identifying the OCR using the set of positions.

61. The method of claim 60, wherein measuring the sizes of cell-free DNA molecules uses the sequence reads.

62. The method of claim 60, wherein the first cutoff value is at least 400 nt.

63. The method of claim 60, wherein the second cutoff value is 500 nt or less.

64. The method of claim 60, wherein identifying the OCR comprises:
identifying a first position of the set of positions with the maximum value of the parameter, and
identifying the OCR as a window of positions around the first position.

65. The method of claim 60, wherein identifying the OCR comprises identifying the OCR as the set of positions.

66. The method of claim 60, wherein identifying the OCR comprises:
identifying a first position of the set of positions between a second position with the maximum value of the parameter and a third position with a second highest value of the parameter, wherein the first position has a local minimum value of the parameter, and
identifying the OCR as a window of positions around the first position.

67. The method of claim 64, wherein the window comprises 1,000 positions or less.

68. The method of claim 64, wherein the window is centered at the first position.

69. The method of claim 60, wherein the reference value is a statistical value calculated using at least a portion of the cell-free DNA molecules.

70. The method of claim 60, wherein the reference value is determined using values of the parameter at sites known to correspond to an OCR.

71. The method of claim 60, wherein the OCR includes a transcription start site (TSS), a DNase1 hypersensitive site (DHS), a CCCTC-binding factor (CTCF) site, or a combination thereof.

72. A method of determining a fractional concentration of cell-free DNA of a first tissue type in a biological sample of a subject, the method comprising:
measuring sizes of cell-free DNA molecules in the biological sample of a subject;
identifying a group of cell-free DNA molecules within an open chromatin region (OCR) of a set of OCRs;
measuring a first amount of the group of cell-free DNA molecules having a size greater than a first cutoff value;
measuring a second amount of the group of cell-free DNA molecules having a size less than a second cutoff value that is equal to or less than the first cutoff value;
determining a value of a parameter using the first amount and the second amount, wherein the parameter reflects a relative amount between the first amount and the second amount;
comparing the value of the parameter with a calibration value obtained from one or more calibration samples having known fractional concentrations of cell-free DNA of the first tissue type; and
estimating the fractional concentration of cell-free DNA of the first tissue type using the comparison.

73. The method of claim 72, wherein the first cutoff value is at least 400 nt.

74. The method of claim 72, wherein the second cutoff value is 500 nt or less.

75. The method of claim 72, wherein a cell-free DNA molecule is within an OCR when an end is located at one or more positions within a window including a center of the OCR.

76. The method of claim 72, wherein a cell-free DNA molecule is within an OCR when at least a specified percentage of the cell-free DNA molecule overlaps with the OCR.

77. The method of claim 76, wherein the specified percentage is 50%, and wherein identifying the group of cell-free DNA molecules comprises identifying cell-free DNA molecules having at least 50% of their length in the OCR.

78. The method of claim 72, wherein each of the one or more OCRs is associated with the first tissue type.

79. The method of claim 72, wherein the first tissue type is tumor cells.

80. The method of claim 72, wherein the OCR is transcription start site (TSS), a DNase1 hypersensitive site (DHS), or a CCCTC-binding factor (CTCF) site.

81. The method of claim 72, wherein the group of cell-free DNA molecules consists of cell-free DNA molecules having a 5′ end at the one or more positions central to the open chromatin region.

82. The method of claim 72, wherein identifying the group of cell-free DNA molecules comprises:
aligning sequence reads of the cell-free DNA molecules to a reference genome to determine genomic coordinates of ends of the cell-free DNA molecules; and
determining, using the genomic coordinates, the cell-free DNA molecules are located in the set of OCRs.

83. The method of claim 82, wherein measuring the sizes of cell-free DNA molecules uses the sequence reads.
